(19) United States
(12) Patent Application Publication
(10) Pub. No.: US 2021/0222171 A1
(43) Pub. Date: Jul. 22, 2021

(54) CRISPR/CAS9 SYSTEMS, AND METHODS OF USE THEREOF

(71) Applicant: AVELLINO LAB USA, INC., Menlo Park, CA (US)

(72) Inventors: Tara MOORE, Ballyclare (GB); Andrew NESBIT, Coleraine (GB); Kathleen CHRISTIE, Boston, MA (US)

(21) Appl. No.: 17/187,666

(22) Filed: Feb. 26, 2021

Related U.S. Application Data
(63) Continuation-in-part of application No. 16/326,908, filed on Feb. 20, 2019.
Application No. 16/326,908 is a continuation of application No. PCT/US2019/048240, filed on Aug. 27, 2019.
(60) Provisional application No. 62/377,586, filed on Aug. 20, 2016.
(60) Provisional application No. 62/462,808, filed on Feb. 23, 2017.
(60) Provisional application No. 62/501,750, filed on May 5, 2017.
(60) Provisional application No. 62/723,450, filed on Aug. 27, 2018.

(51) Int. Cl. C12N 15/113; C12N 9/22

(52) U.S. Cl. CPC C12N 15/113; C12N 2310/20; C12N 9/22

(57) ABSTRACT

The present disclosure relates to Clustered Regularly Interspaced Short Palindromic Repeats (CRISPR)/CRISPR associate protein 9 (Cas9) systems, and methods of use thereof for gene editing.

### RELATED APPLICATIONS

This application is a continuation-in-part of U.S. patent application Ser. No. 16/326,908 filed Feb. 20, 2019, which is a national stage application under 35 U.S.C. 371 of the international application No. PCT/US2017/047861 filed Aug. 21, 2017, which claims priority to U.S. Provisional Patent Application No. 62/377,586 filed Aug. 20, 2016, U.S. Provisional Patent Application No. 62/462,808 filed Feb. 23, 2017, and U.S. Provisional Patent Application No. 62/501,750 filed May 5, 2017, all of which are incorporated by reference herein in their entireties. This application is a continuation application of the international application No. PCT/US2019/048240 filed Aug. 27, 2019, which claims priority to U.S. Provisional Patent Application No. 62/723,450 filed Aug. 27, 2018, both of which are incorporated by reference herein in their entireties.

### SEQUENCE LISTING SUBMISSION VIA EFS-WEB

A computer readable text file, entitled “SequenceListing.txt,” created on or about Feb. 26, 2021 with a file size of about 1,154 KB contains the sequence listing for this application and is hereby incorporated by reference in its entirety.

### FIELD OF THE INVENTION

The present disclosure relates to Clustered Regularly Interspaced Short Palindromic Repeats (CRISPR)/CRISPR associated protein 9 (Cas9) systems, and methods of use thereof for gene editing or preventing, ameliorating or treating a disease associated with a gene mutation or single nucleotide polymorphism (SNP) in a subject.

### BACKGROUND OF THE INVENTION

The majority of corneal dystrophies are inherited in an autosomal dominant fashion with a dominant-negative pathomechanism. For some genes, for example TGFBI and KRT12, it has been shown that they are haplosufficient; meaning one functional copy of the gene is sufficient to maintain normal function. By using siRNA that specifically targets the mutant allele, it is possible to overcome the dominant-negative effect of the mutant protein and restore normal function to cells in vitro. Whereas the effects of siRNA are transient, lasting only as long as the siRNA is present in the cell at high enough concentrations; CRISPR/Cas9 gene editing offers the opportunity to permanently modify the mutant allele.

The discovery of a simple endogenous bacterial system for catalytically cleaving double-stranded DNA has revolutionized the field of therapeutic gene editing. The Type II Clustered Regularly Interspaced Short Palindromic Repeats (CRISPR)/CRISPR associated protein 9 (Cas9) is a programmable RNA guided endonuclease, which has recently been shown to be effective at gene editing in mammalian cells (Hsu P D, Lander E S, Zhang F. Development and applications of CRISPR-Cas9 for genome engineering. Cell 2014; 157: 1262-1278). This highly specific and efficient RNA-guided DNA endonuclease may be of therapeutic importance in a range of genetic diseases. The CRISPR/Cas9 system relies on a single catalytic protein, Cas9 that is guided to a specific DNA sequence by 2 RNA molecules; the tracrRNA and the crRNA (Hsu P D, Lander E S, Zhang F. Development and applications of CRISPR-Cas9 for genome engineering. Cell 2014; 157: 1262-1278). Combination of the tracrRNA/crRNA into a single guide RNA molecule (sgRNA) (Shalem O, Sanjana N E, Hartenian E, Shi X, Scott D A, Mikkelsen T S et al. Genome-scale CRISPR-Cas9 knockout screening in human cells. Science 2014; 343: 84-87; Wang T, Wei J J, Sabatini D M, Lander E S. Genetic screens in human cells using the CRISPR-Cas9 system. Science 2014; 343: 80-84) has led to the rapid development of gene editing tools potentially specific for any target within the genome. Through the substitution of a nucleotide sequence within the sgRNA, to one complimentary to a chosen target, a highly specific system may be generated in a matter of days. One caveat of this system is that the endonuclease requires a protospacer adjacent motif (PAM), located immediately at the 3′ end of the sgRNA binding site. This PAM sequence is an invariant part of the DNA target but not present in the sgRNA, while its absence at the 3′ end of the genomic target sequence results in the inability of the Cas9 to cleave the DNA target (Westra E R, Semenova E, Datsenko K A, Jackson R N, Wiedenheft B, Severinov K et al. Type I-E CRISPR-cas systems discriminate target from non-target DNA through base pairing-independent PAM recognition. PLoS Genet 2013; 9: e1003742). This distinction is important as the mutation directly in a PAM-specific approach, or nearby SNPs may be targeted. One SNP allele will represent a PAM site, while the other allele does not. This allows us to discriminate between the two chromosomes.

### SUMMARY OF THE INVENTION

In one aspect, the present disclosure describes the potential of utilizing the PAM-generating mutations in introns of a disease-causing gene. For example, the PAM-generating mutations are in adjacent introns of a gene having a disease-causing mutation, and the disease-causing mutation is in exon in between the adjacent introns.

By utilizing guide sequences that bind adjacent to the PAM sequences in the introns, Cas9 nuclease may cleave a gene at two intronic sites, between which an exon containing a disease-causing mutation exists, thereby eliminating the disease-causing exon and knocking out the mutated allele. In another aspect, the CRISPR/Cas9 system utilizing the PAM-generating mutations or SNPs in introns may be used to treat corneal dystrophies, for example, including corneal dystrophy associated with R124H granular corneal dystrophy type 2 mutation.

In one aspect, the present disclosure is related to an sgRNA pair designed for CRISPR/Cas9 system. For example, the sgRNA pair may comprise (i) a first sgRNA comprising (a) a first crRNA sequence for a first protospacer adjacent motif (PAM) generating mutation or single-nucleotide polymorphism (SNP) in a first intron at 3′-end side of an exon comprising a disease-causing mutation or SNP in cis, and (b) a tracrRNA sequence, in which the first crRNA sequence and the tracrRNA sequence do not naturally occur together; and (ii) a second sgRNA comprising (a) a second crRNA guide sequence for a second PAM generating mutation or SNP in a second intron at 5′-end side of the exon comprising the disease-causing mutation or SNP in cis; (b) a tracrRNA sequence, in which the second crRNA sequence and the tracrRNA sequence do not naturally occur together. In some embodiments, the CRISPR/Cas9 system is for preventing, ameliorating or treating corneal dystrophies. In additional embodiments, the exon and the first and second introns are of TGFBI gene. In further embodiments, at least one of the first and second crRNA sequences comprises a nucleotide sequence selected from the group consisting of guide sequences shown in Table 3.

In one aspect, the present disclosure is related to an engineered Clustered Regularly Interspaced Short Palindromic Repeats (CRISPR)/CRISPR associate protein 9 (Cas9) system comprising at least one vector comprising a nucleotide molecule encoding Cas9 nuclease and the sgRNA pair described herein, wherein the Cas9 nuclease and said sgRNA pair in the vector do not naturally occur together. In some embodiments, the at least one vector includes a single-stranded oligonucleotide containing (i) a first portion corresponding to (or complementary to) the sequence of the first intron on the 3′-end side of a cleavage site associated with the first PAM and (ii) a second portion corresponding to (or complementary to) the sequence of the second intron on the 5′-end side of a cleavage site associated with the second PAM. In some embodiments, the first portion has 50 nucleotides and the second portion has 50 nucleotides. In some embodiments, the first portion is adjacent to the second portion.

In one aspect, the present disclosure is related to methods of preventing, ameliorating, or treating corneal dystrophy, the method comprising administering to the subject an engineered CRISPR/Cas9 system comprising at least one vector comprising at least two different CRISPR targeting RNA (crRNA) sequences or single guide RNA (sgRNA) sequences. In one aspect, the present disclosure is related to methods of preventing, ameliorating, or treating corneal dystrophy in a subject, comprising administering to the subject an engineered CRISPR/Cas9 system comprising at least one vector comprising (i) a nucleotide molecule encoding Cas9 nuclease; (ii) a first sgRNA comprising a first crRNA sequence that hybridizes to a nucleotide sequence complementary to a first target sequence, the first target sequence being adjacent to the 5′-end of a first protospacer adjacent motif (PAM) in a first intron at the 3′-end side of an exon comprising a disease-causing mutation or SNP in cis, wherein the first target sequence or the first PAM comprises a first ancestral variation or SNP site; and (iii) a second sgRNA comprising a second crRNA sequence that hybridizes to a nucleotide sequence complementary to a second target sequence, the second target sequence being adjacent to the 5′-end of a second PAM in a second intron at the 5′ side of the exon comprising the disease-causing mutation or SNP in cis, wherein the second target sequence or the second PAM comprises a second ancestral variation or SNP site, wherein the at least one vector does not have a nucleotide molecule encoding Cas9 nuclease and a sgRNA sequence that naturally occur together. In some embodiments, the corneal dystrophy is associated with R124H granular corneal dystrophy type 2 mutation.

In some embodiments, the first PAM comprises the first ancestral variation or SNP site and/or the second PAM comprises the second ancestral variation or SNP site. In some embodiments, the first crRNA sequence comprises the first target sequence, and the second crRNA sequence comprises the second target sequence. In further embodiments, the first crRNA sequence is from 17 to 24 nucleotide long; and/or the second crRNA sequence is from 17 to 24 nucleotide long.

In some embodiments, the first and/or second PAMs and the Cas9 nuclease are from Streptococcus or Staphylococcus. In additional embodiments, the first and second PAMs are both from Streptococcus or Staphylococcus. In some embodiments, each of the first and second PAMs independently consists of NGG or NNGRRT, wherein N is any of A, T, G, and C, and R is A or G. In some embodiments, the administration comprises injecting the engineered CRISPR/Cas9 system into the subject. In additional embodiments, the administering comprises introducing the engineered CRISPR/Cas9 system into a cell containing and expressing a DNA molecule having the target sequence.

In some embodiments, the disease is associated with the SNP; the first target sequence or the first PAM comprises the first ancestral SNP site; and/or the second target sequence or the second PAM comprises the second ancestral SNP site. In additional embodiments, the target sequence or the PAM comprises a plurality of mutation or SNP sites. In some embodiments, the subject is human.

In some embodiments, the methods described herein further comprises, prior to administering to the subject the engineered CRISPR/Cas9 system, obtaining genomic or sequence information of the subject; and selecting the first crRNA sequence and/or the second crRNA sequence based on the genomic or sequence information of the subject. In additional embodiments, the genomic or sequence information of the subject includes whole or partial genome sequence information of the subject.

In some embodiments, the first crRNA sequence hybridizes to the nucleotide sequence so that the Cas9 nuclease cleaves at a first cleaving site that is adjacent to the first ancestral variation or SNP site; and/or the second crRNA sequence hybridizes to the nucleotide sequence so that the Cas9 nuclease cleaves at a second cleaving site that is adjacent to the second ancestral variation or SNP site. In additional embodiments, the first crRNA sequence hybridizes to the nucleotide sequence so that the Cas9 nuclease cleaves only at the first cleaving site that is adjacent to the first ancestral variation or SNP site; and/or the second crRNA sequence hybridizes to the nucleotide sequence so that the Cas9 nuclease cleaves only at the second cleaving site that is adjacent to the second ancestral variation or SNP site. In further embodiments, the first crRNA sequence is configured to reduce cleaving of the genome of the subject at a site other than a first cleaving site compared to other crRNA sequences hybridizing to the nucleotide sequence complementary to the first target sequences; and/or the second crRNA sequence is configured to reduce cleaving of the genome of the subject at a site other than a second cleaving site compared to other crRNA sequences hybridizing to the nucleotide sequence complementary to the second target sequences. In yet further embodiments, the first crRNA sequence is configured to reduce cleaving of a gene, in trans, that corresponds to a gene causing the disease in cis compared to other crRNA sequences hybridizing to the nucleotide sequence complementary to the first target sequences; and/or the second crRNA sequence is configured to reduce cleaving of a gene, in trans, that corresponds to the gene causing the disease in cis compared to other crRNA sequences hybridizing to the nucleotide sequence complementary to the second target sequences.

In some embodiments, the selected first crRNA sequence is configured to cause cleaving at a first cleaving site, within genome of the subject, that is adjacent to the first ancestral variation or SNP site; and/or the selected second crRNA sequence is configured to cause cleaving at a second cleaving site, within the genome of the subject, that is adjacent to the second ancestral variation or SNP site. In additional embodiments, the selected first crRNA sequence is configured to cause cleaving only at the first cleaving site; and/or the selected second crRNA sequence is configured to cause cleaving only at the second cleaving site. In further embodiments, the first crRNA sequence hybridizes to the nucleotide sequence complementary to the first target sequence in trans with the disease-causing mutation or SNP, said first target sequence in trans not being adjacent to the 5′-end of a PAM; and/or the second crRNA sequence hybridizes to the nucleotide sequence complementary to the second target sequence in trans with the disease-causing mutation or SNP, said second target sequence not being adjacent to the 5′-end of a PAM.

### BRIEF DESCRIPTION OF THE DRAWINGS

FIG. 1 illustrates an example of a sgRNA sequence, nucleotide and amino acid sequences of Cas9 nuclease from Streptococcus pyogenes (Spy) and Staphylococcus aureus (Sau).

FIG. 2 illustrates an example of a dual-cut approach using intronic PAM sites. Two separate guides are introduced, and Cas9 generates a double stranded break (DSB) at two sites. Repair of this doubly cut region will result in an excision of the region between the two breaks. The deletion encompasses the exonic coding region of the gene shown by the yellow boxes in this figure.

FIG. 3 illustrates an embodiment in which a sgRNA utilizing a flanking SNP within the PAM site is designed in the first intron. Additionally, a sgRNA common to both the wild-type and mutant allele is designed in the second intron. In the wild-type allele the single sgRNA causes NHEJ in the second intron, which may have no functional effect. However, in the mutant allele, the sgRNA utilizing the flanking SNP derived PAM and the common sgRNA result in a large deletion that results in a knockout of the mutant allele.

FIG. 4 illustrates all SNPs in TGFBI with a MAF of >10% that generate a novel PAM. The numbered boxes indicate the exons within TGFBI. The hotspots in TGFBI, where multiple disease-causing mutations are found, are shown by the red boxes. The blue arrows indicate the position of a SNP that generates a novel PAM. The novel PAM is shown for each arrow, with the required variant highlighted in red.

FIG. 5 depicts experimental results from using an exemplary lymphocyte cell line derived from a patient with a R124H granular corneal dystrophy type 2 mutation that was nucleofected with CRISPR/Cas9 and sgRNA. The guide utilized the novel PAM that is generated by the rs3805700 SNP. This PAM is present on the same chromosome as the patients R124H mutation but does not exist on the wild-type chromosome. Following cell sorting, single clones were isolated to determine whether indels had occurred. Six of the single clones had the unedited wild-type chromosome, indicating stringent allele-specificity of this guide. Four of the isolated clones had the mutant chromosome, and three of these exhibited edits indicating a 75% editing efficiency of the mutant chromosome. Two of the three clones exhibited indels that are frame-shifting. Therefore, at least 66.66% of the edits induced gene disruption.

FIG. 6 shows the results from a dual-guide approach.

FIG. 7, on the right, illustrates that using the original clonal isolation of single alleles, a 565 bp deletion encompassing both PAM sites was confirmed. The deletion is shown in red with the PAM sites highlighted in blue. On the left, FIG. 7 also illustrates the two guides cutting at their target sites, the region between these cuts being excised upon repair, and the genomic region after repair.

FIGS. 8-23 illustrate exemplary common guides in intronic regions of TGFBI gene.

FIG. 24 illustrates (a) locations of exemplary nine SNPs in intronic PAM sites to be used in certain dual-cut examples described herein, (b) in vitro experimental results of using the exemplary nine SNPs, and (c) experimental results of using the exemplary nine SNPs in lymphocyte cell line.

FIG. 25 describes experimental results from transfecting exemplary complexes of Cas9 and guides based on the nine SNPs into a lymphocyte cell line generated from a R124H GCD2 corneal dystrophy patient.

FIG. 26 depicts locations of additional exemplary common intronic guides, CI-1 through CI-4. These guides are configured to cause cleavage of both alleles (by Cas9). As described herein, any of these guides can be used alongside an allele-specific ASNIP guide to cause a dual-cut that has a functional effect when both cuts happen for any particular allele.

FIG. 27 depicts six exemplary different dual combinations tested and their associated deletion.

FIGS. 28-33 illustrate experimental results from transfecting exemplary complexes of guides of FIG. 27 and Cas9 into R124H patient-derived cells.

FIG. 34 illustrates that addition of the 50-50 bp ssODN improved the efficiency of the dual cut.

FIG. 35 illustrates that, in a wild-type (WT) allele, a single cut and a repair in an intronic region have no functional effect.

FIG. 36 shows experimental results confirming that the dual-cut indeed occurred using the exemplary complexes.

FIG. 37 illustrates example SNPs containing a PAM on only one allele.

FIG. 38 illustrates example SNPs associated with a PAM on only one allele that lie in cis with the patient's R124H mutations.

FIG. 39 illustrates example guide pairs used in an experiment.

FIG. 40 illustrates example guide pairs with large distances between the two guides.

FIG. 41 illustrates example guide pairs with smaller distances between the two guides.

FIG. 42 illustrates example guide pairs used in an experiment.

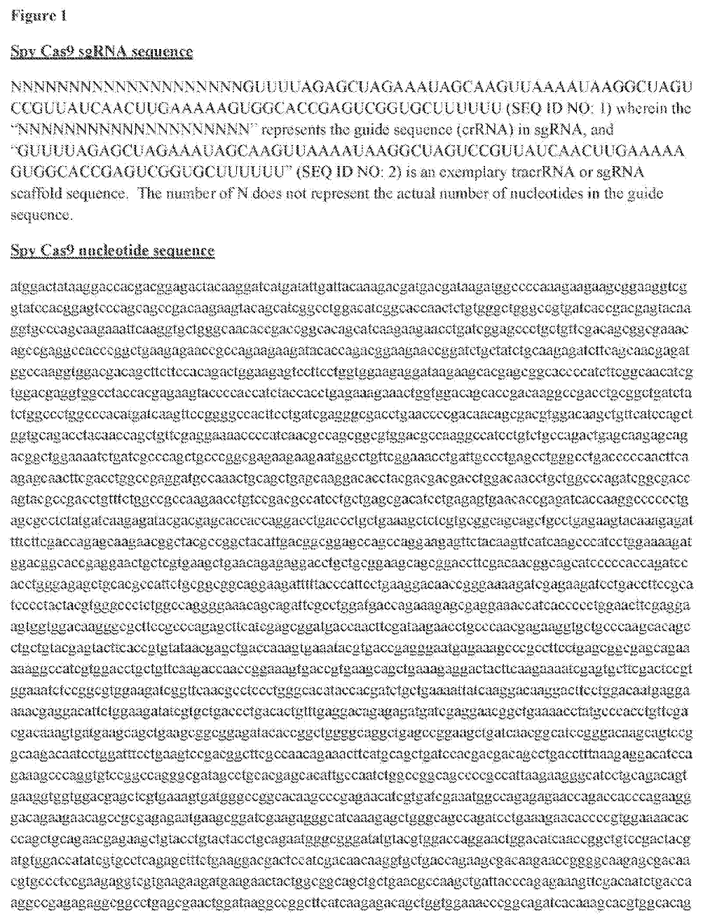

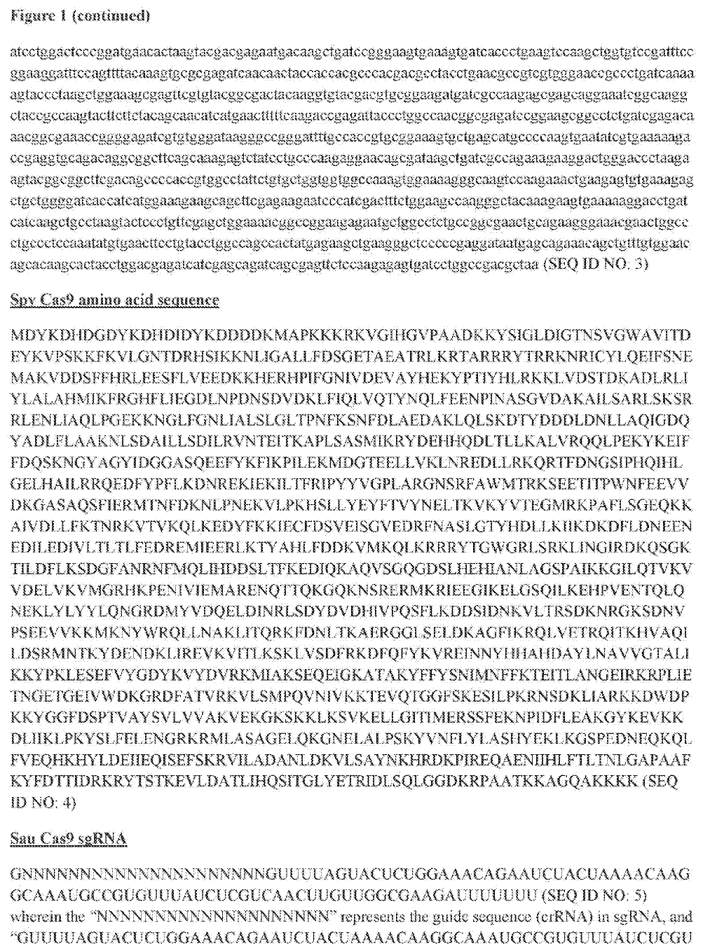

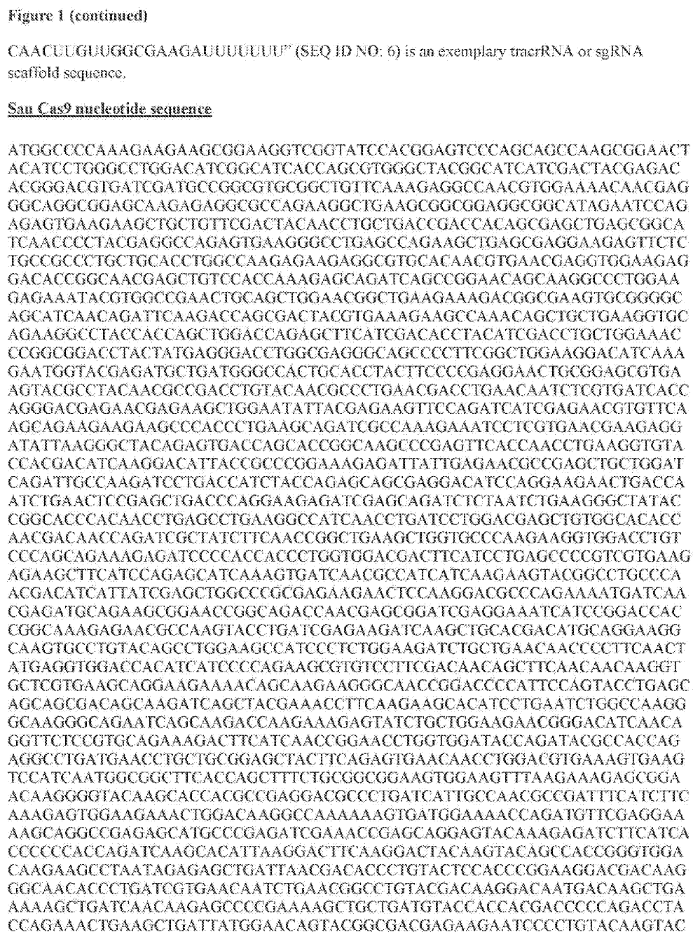

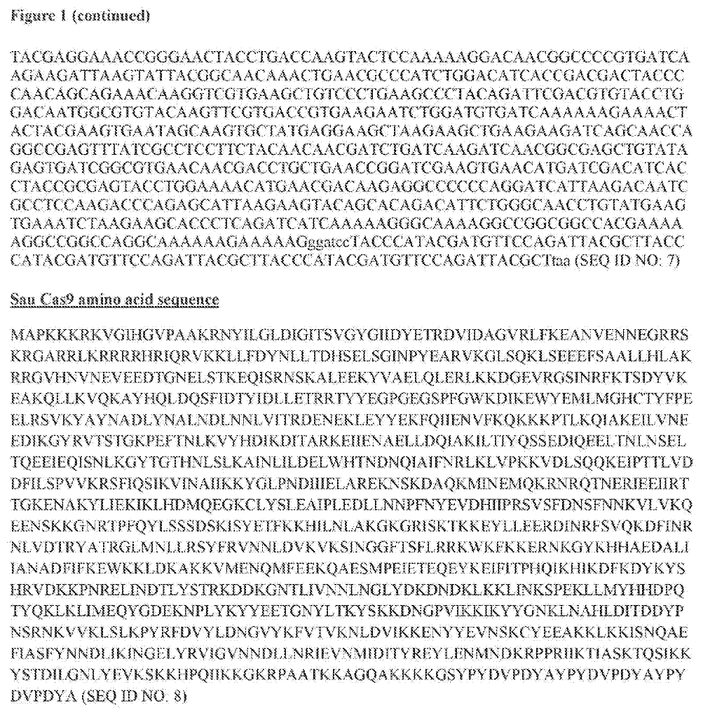

### DETAILED DESCRIPTION OF THE INVENTION

As used throughout, ranges are used as shorthand for describing each and every value that is within the range. Any value within the range can be selected as the terminus of the range. In addition, all references cited herein are hereby incorporated by reference in their entireties for all purposes. In the event of a conflict in a definition in the present disclosure and that of a cited reference, the present disclosure controls.

In one aspect, the present disclosure is related to an sgRNA pair designed for CRISPR/Cas9 system. For example, the sgRNA pair may comprise (i) a first sgRNA comprising (a) a first crRNA sequence for a first protospacer adjacent motif (PAM) generating mutation or single-nucleotide polymorphism (SNP) in a first intron at 3′-end side of an exon comprising a disease-causing mutation or SNP in cis, and (b) a tracrRNA sequence, in which the first crRNA sequence and the tracrRNA sequence do not naturally occur together; and (ii) a second sgRNA comprising (a) a second crRNA guide sequence for a second PAM generating mutation or SNP in a second intron at 5′-end side of the exon comprising the disease-causing mutation or SNP in cis; (b) a tracrRNA sequence, in which the second crRNA sequence and the tracrRNA sequence do not naturally occur together. In some embodiments, the CRISPR/Cas9 system is for preventing, ameliorating or treating corneal dystrophies. In some embodiments, the corneal dystrophy is associated with R124H granular corneal dystrophy type 2 mutation. In additional embodiments, the exon and the first and second introns are of TGFBI gene. In further embodiments, at least one of the first and second crRNA sequences comprises a nucleotide sequence selected from the group consisting of guide sequences shown in Table 3.

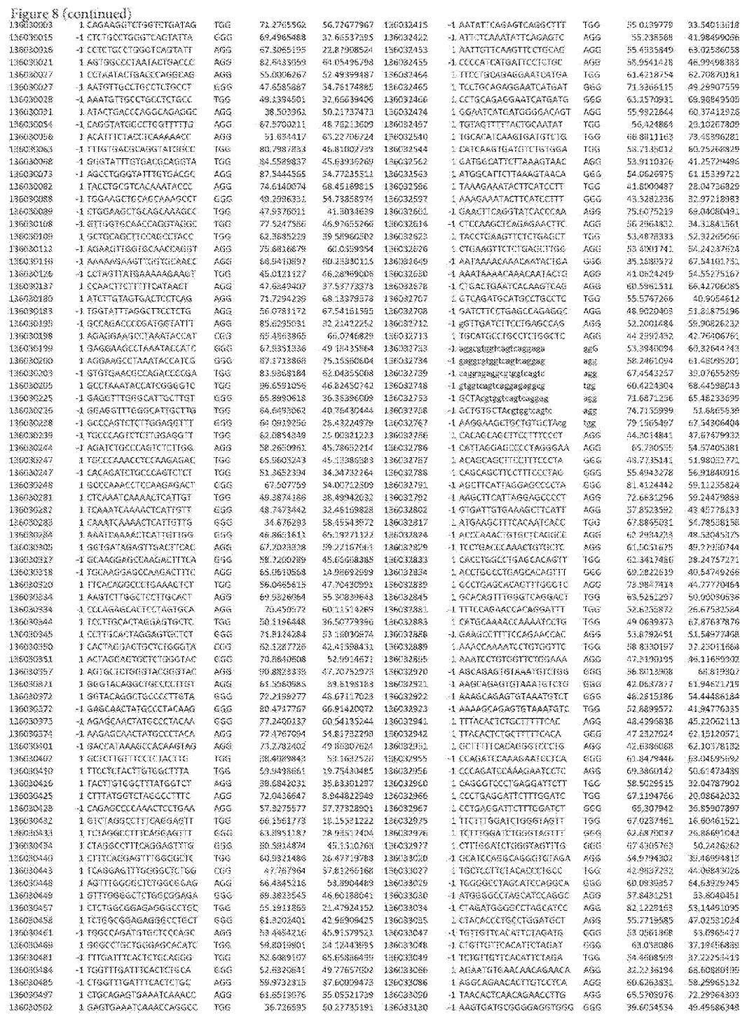

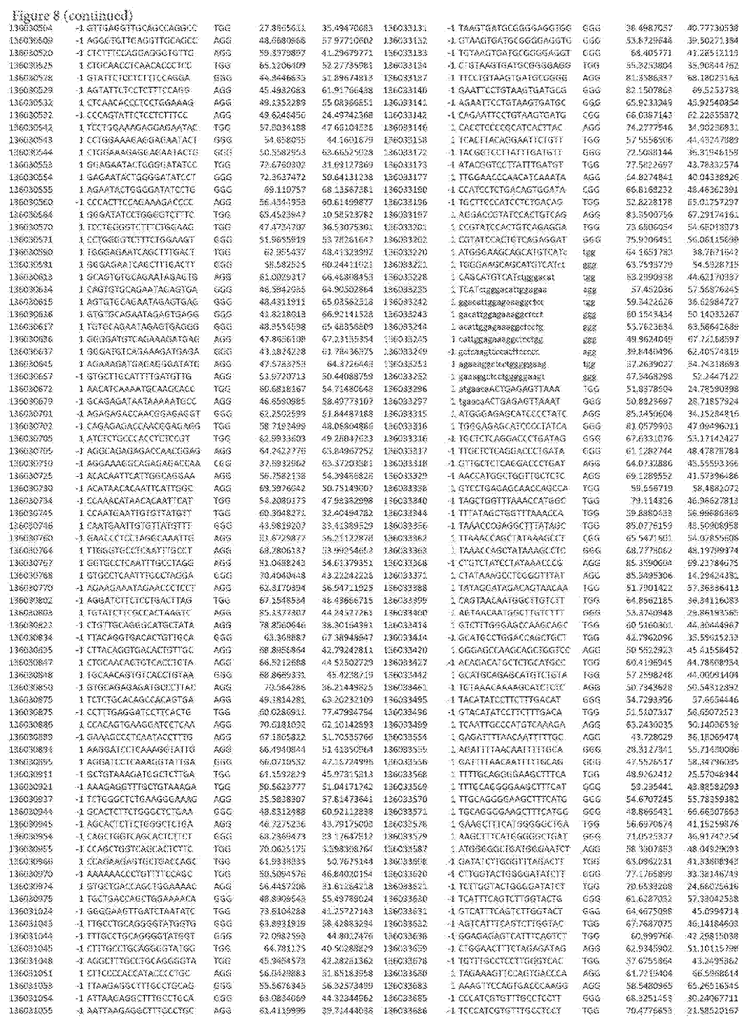

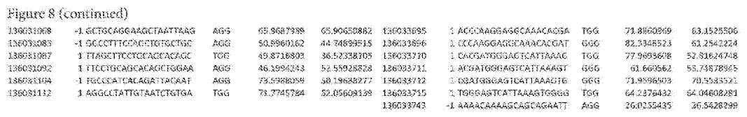

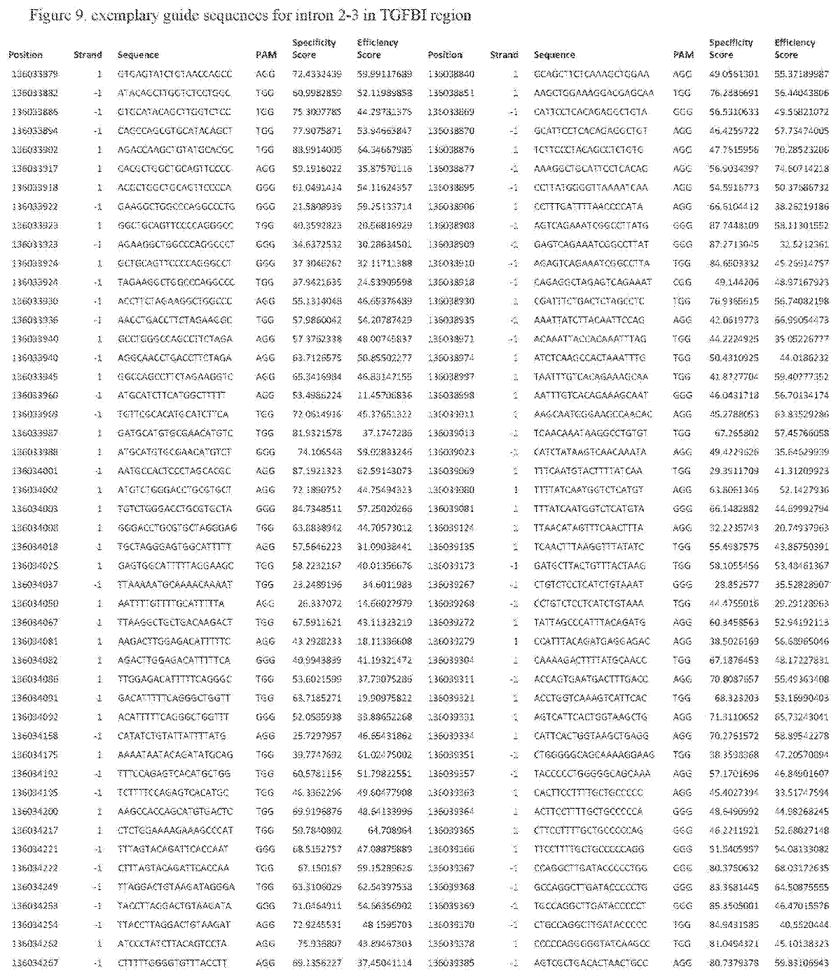

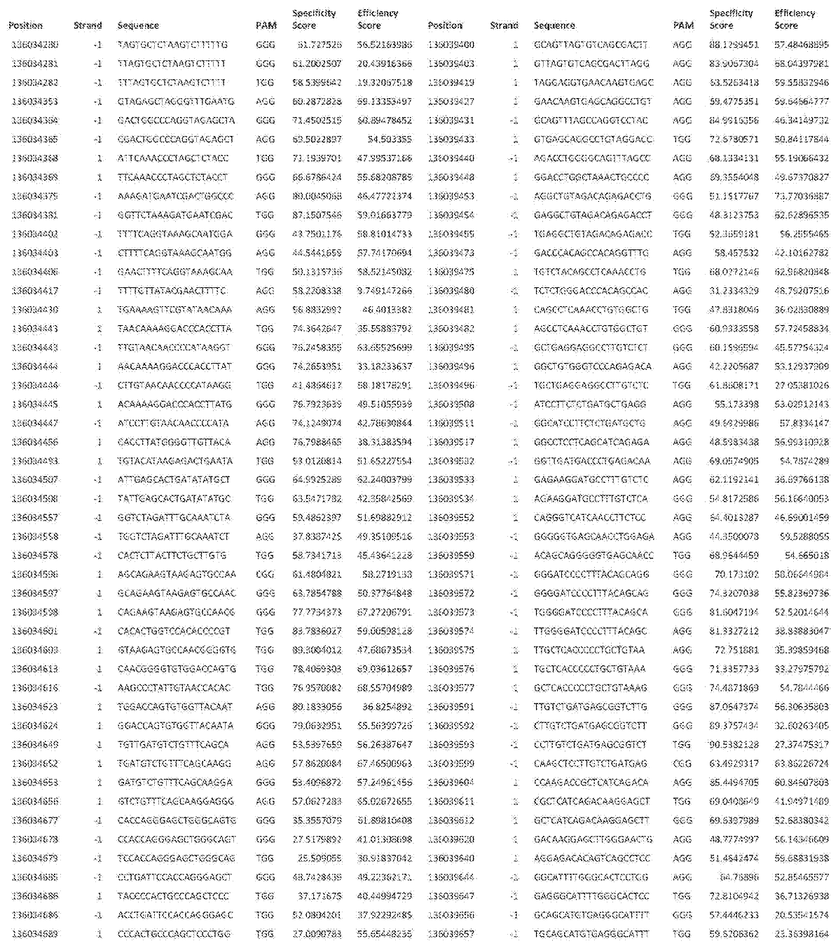

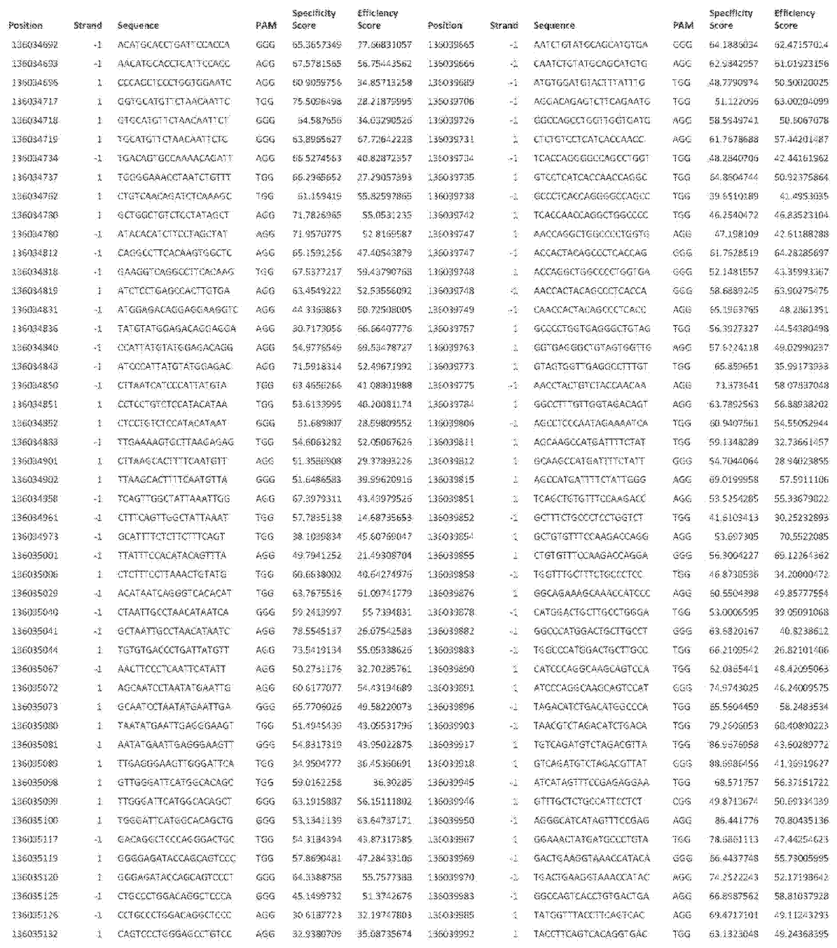

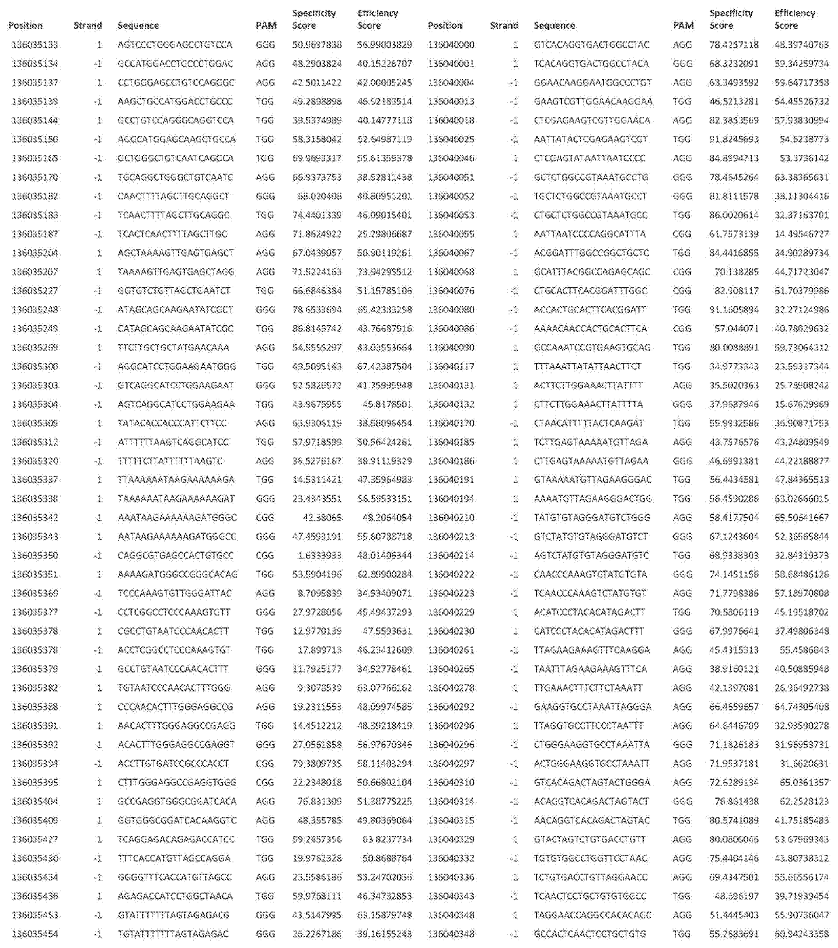

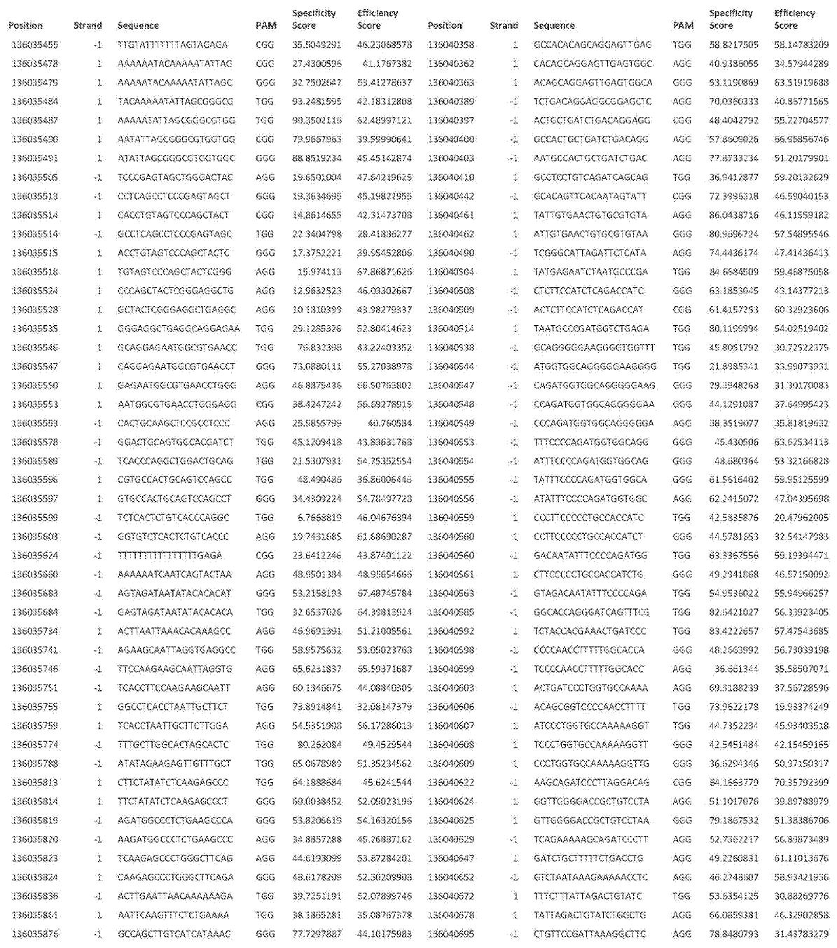

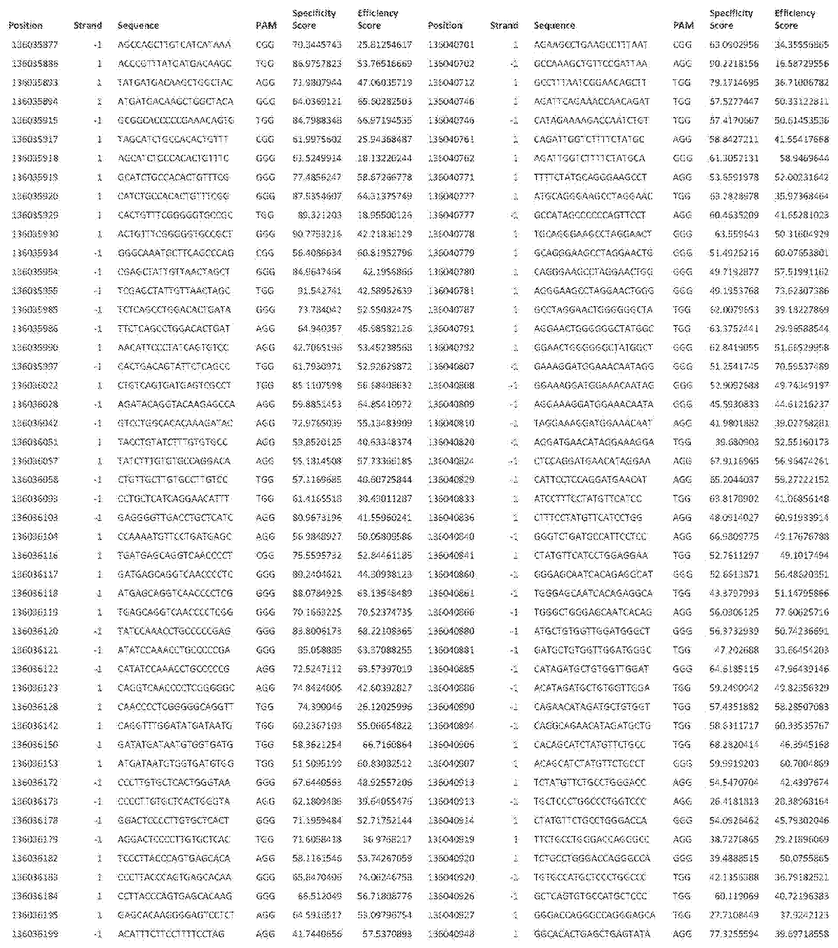

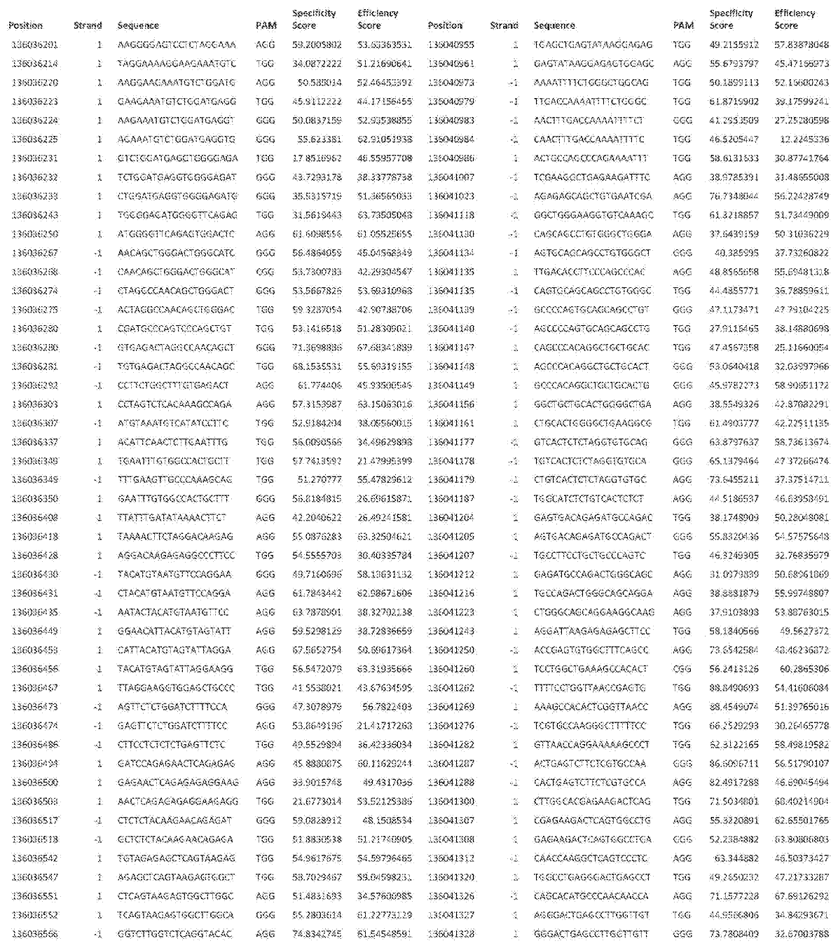

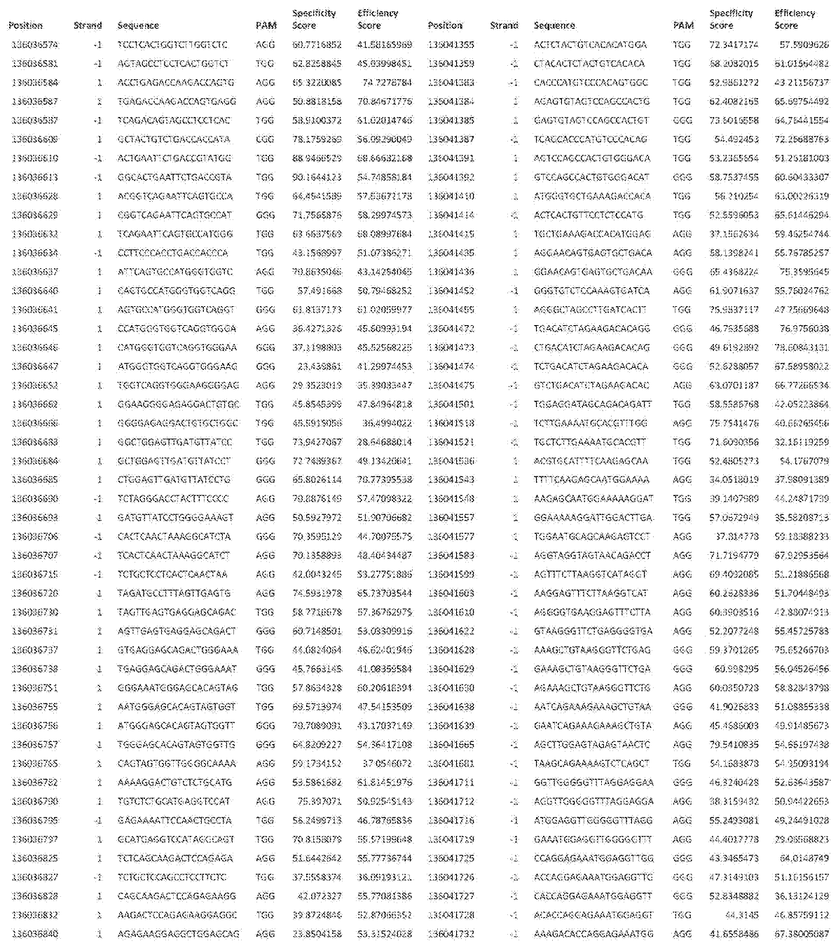

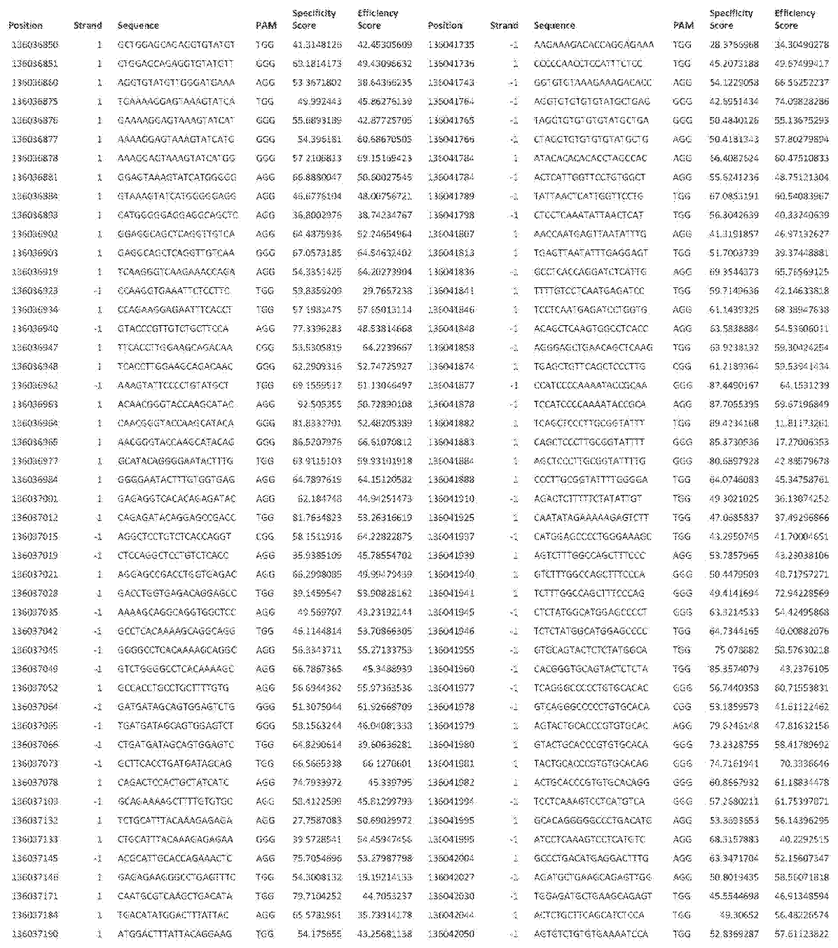

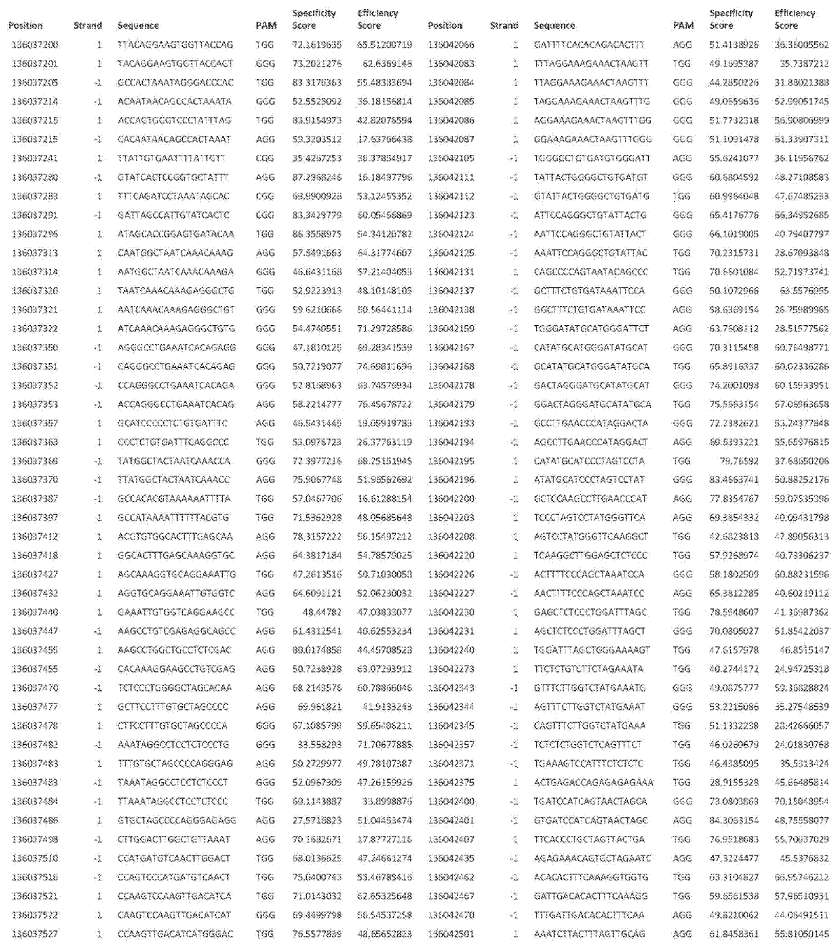

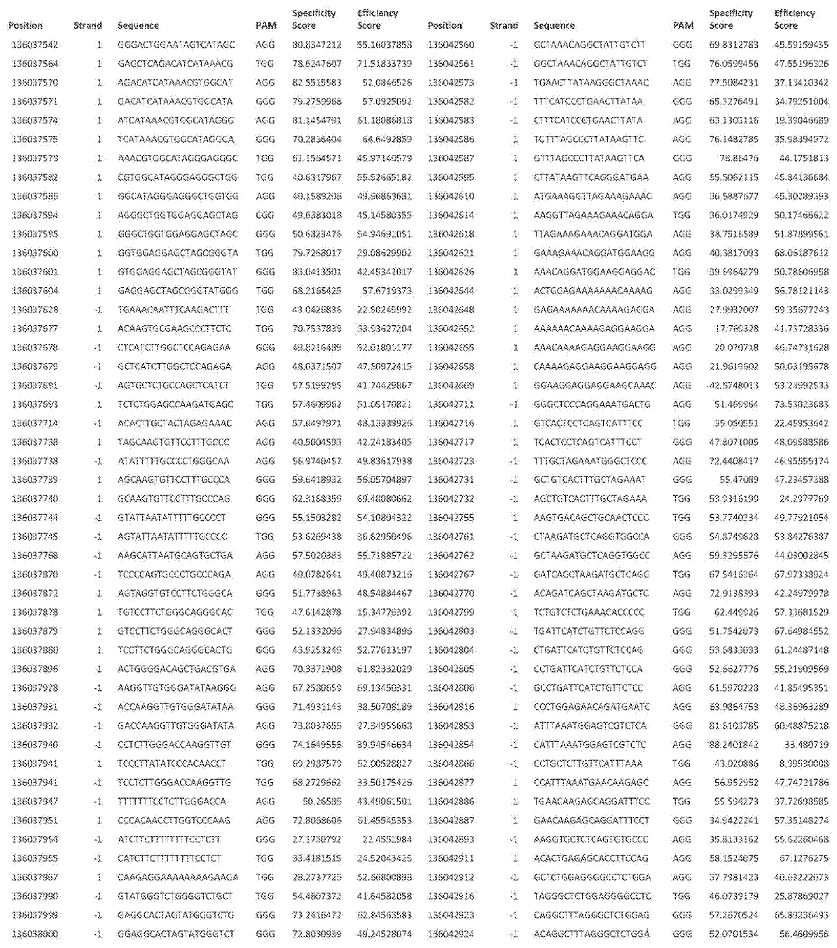

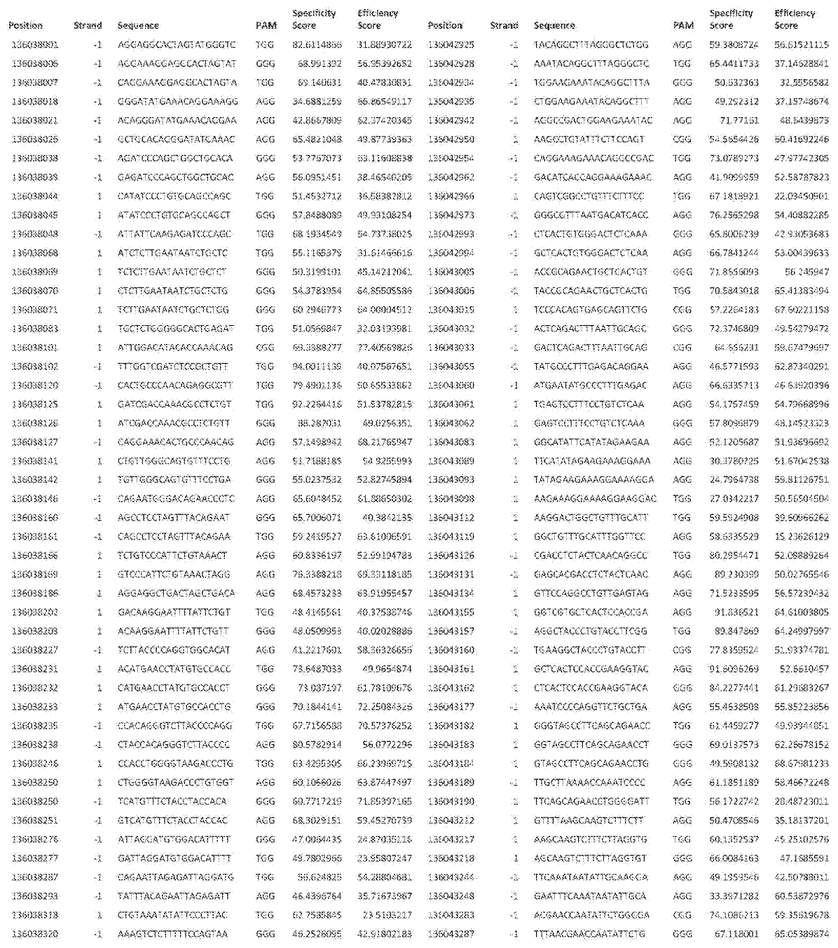

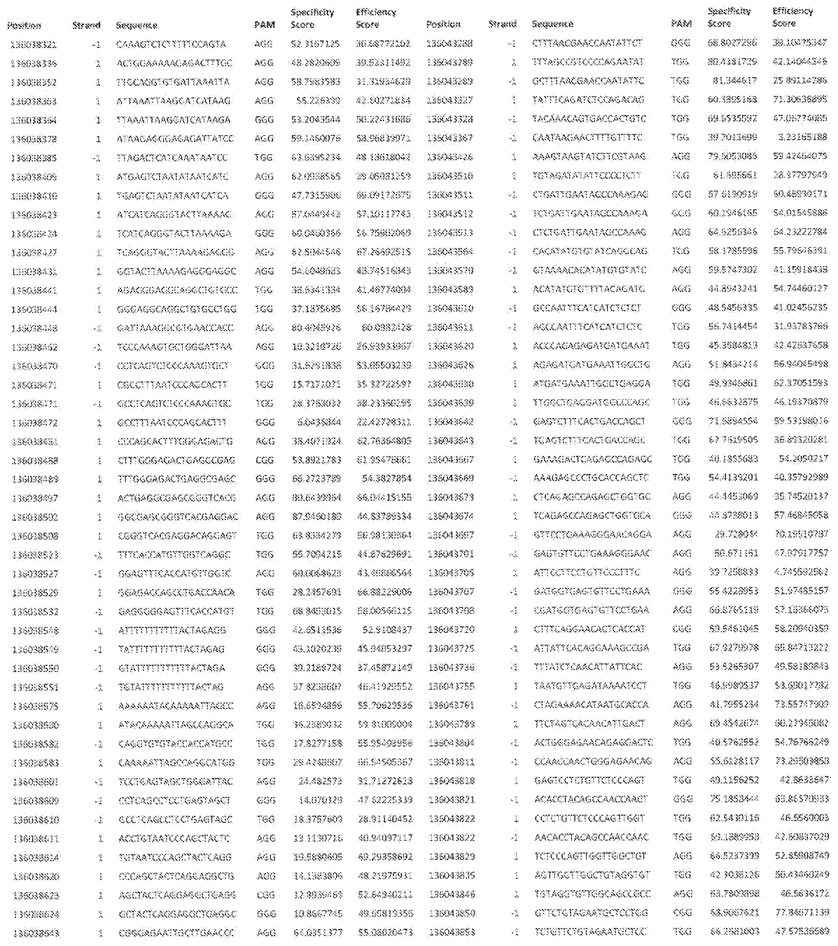

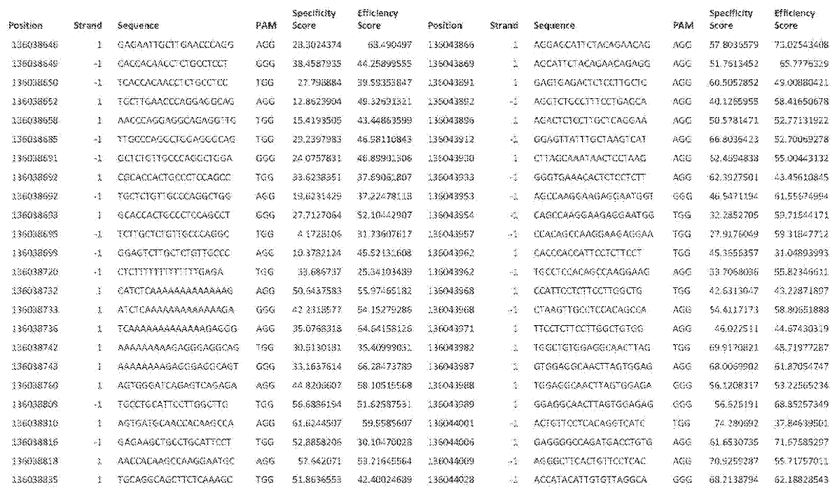

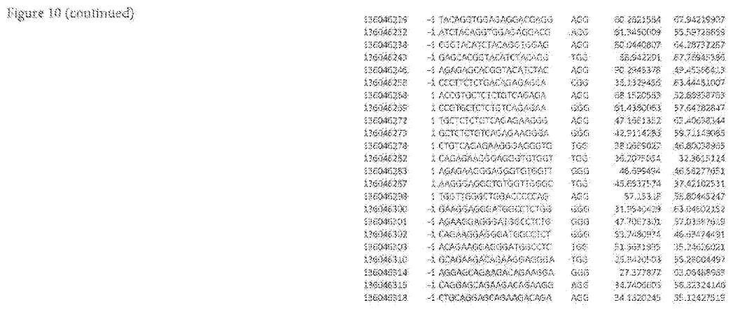

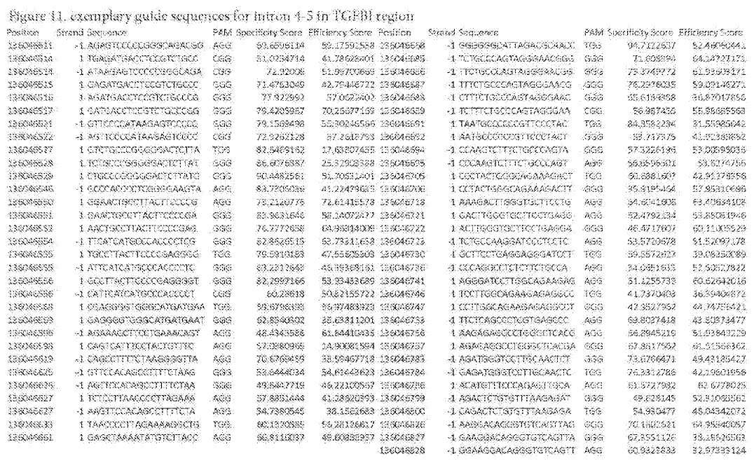

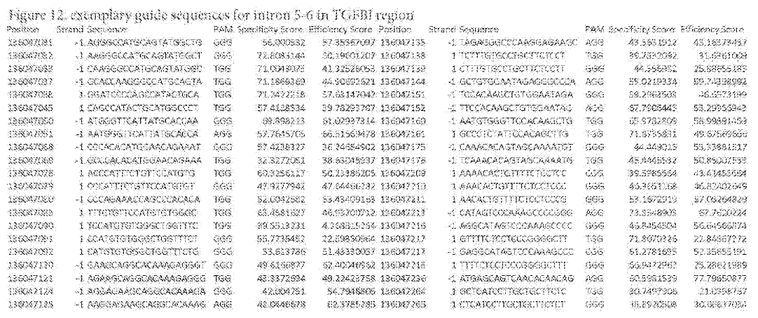

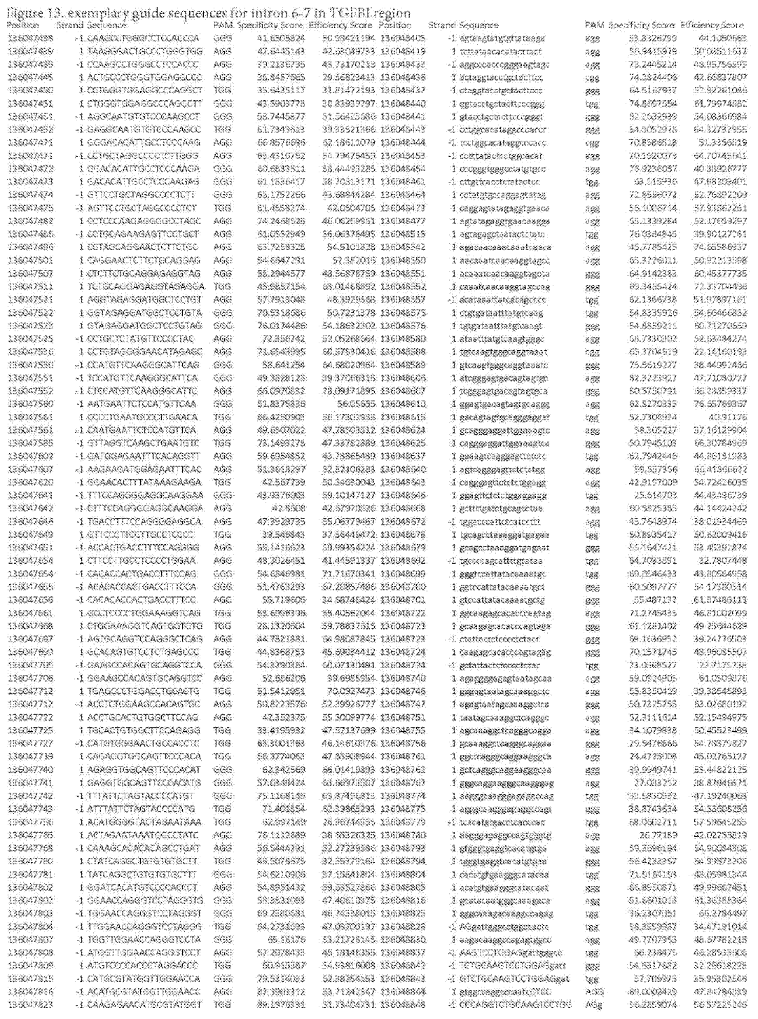

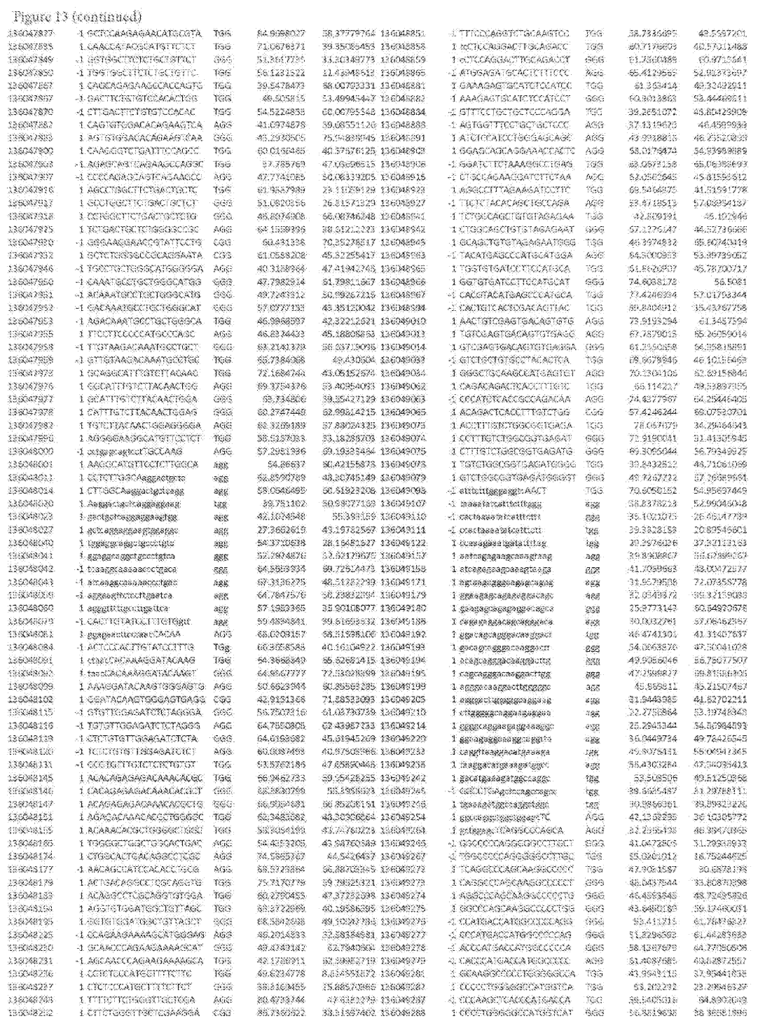

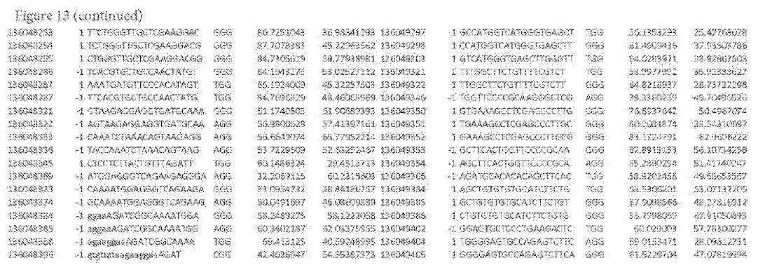

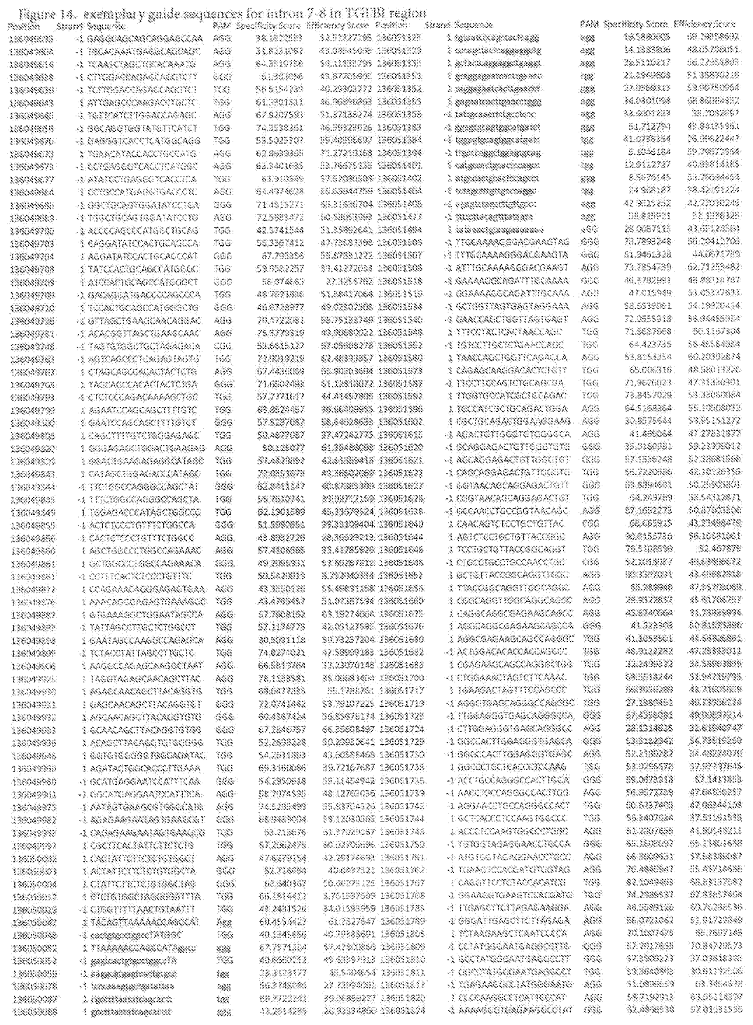

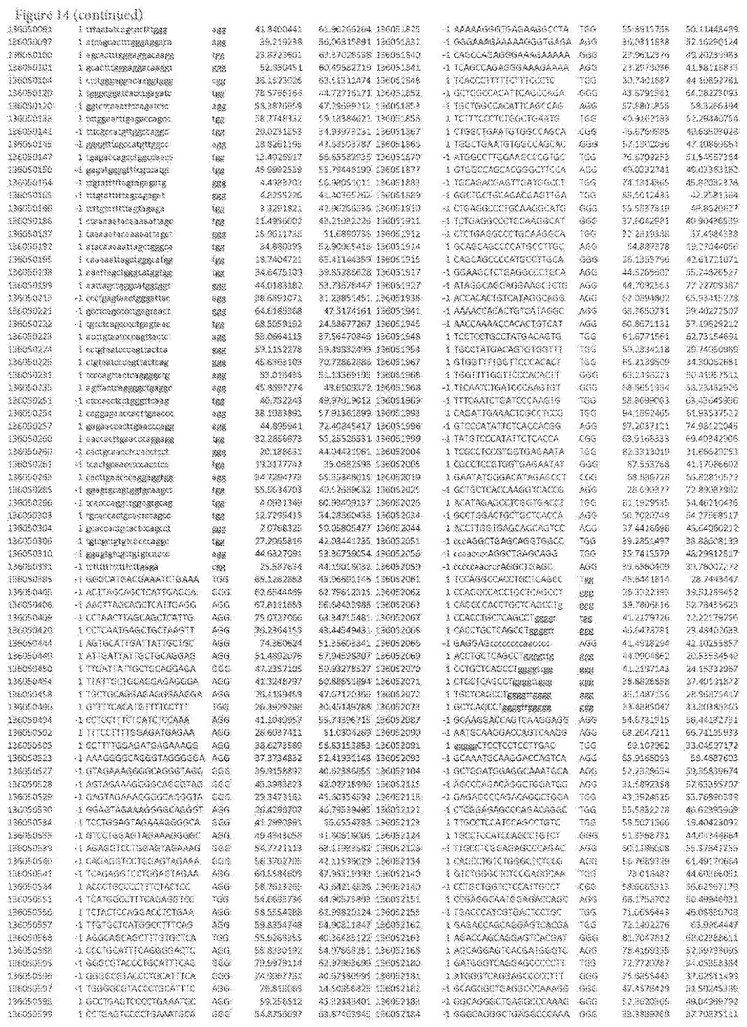

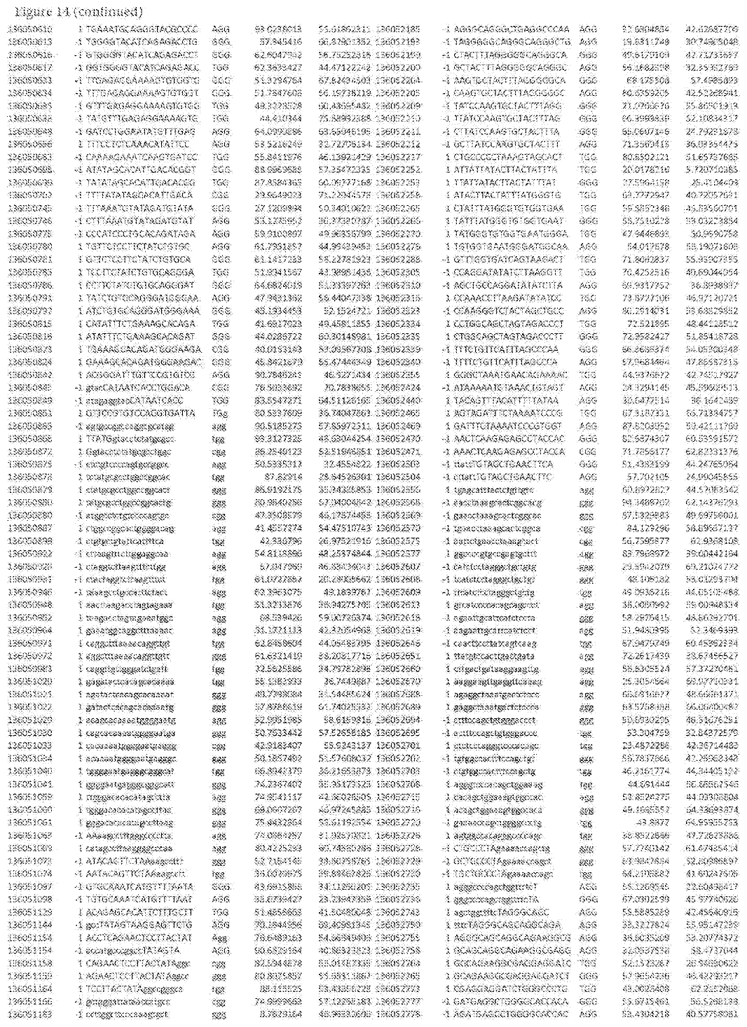

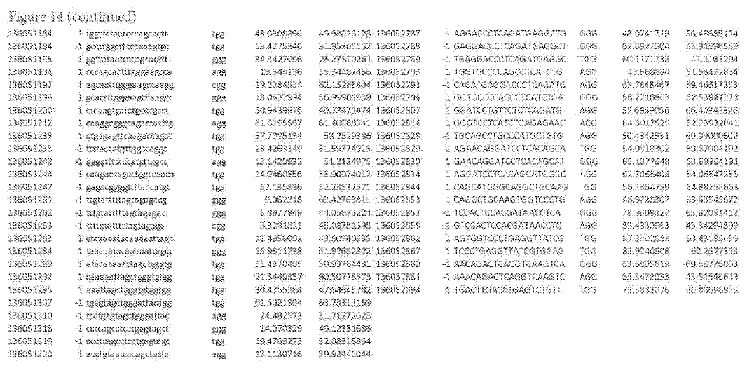

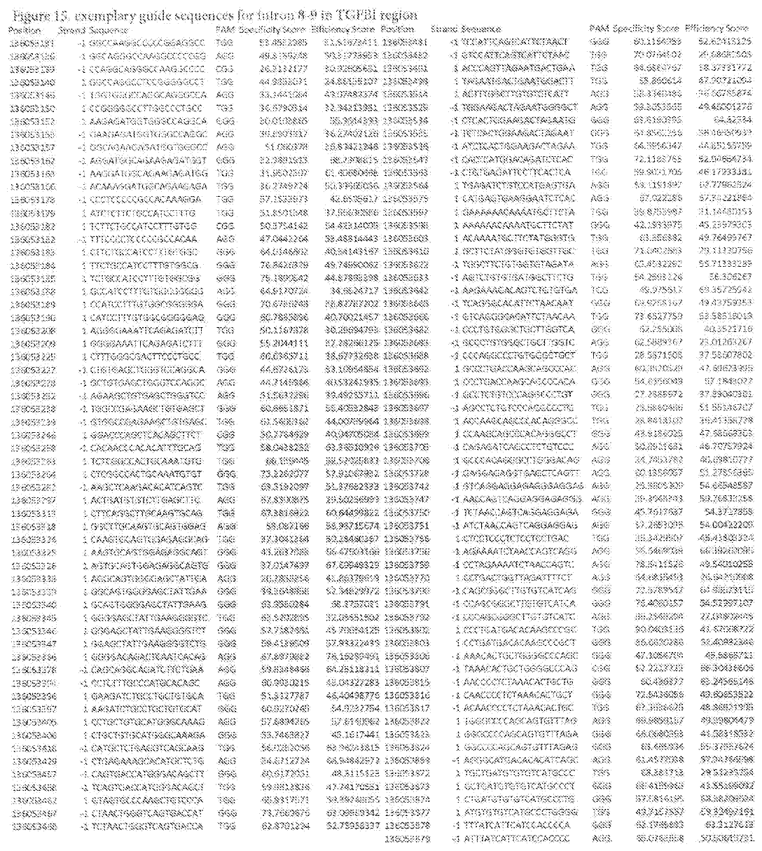

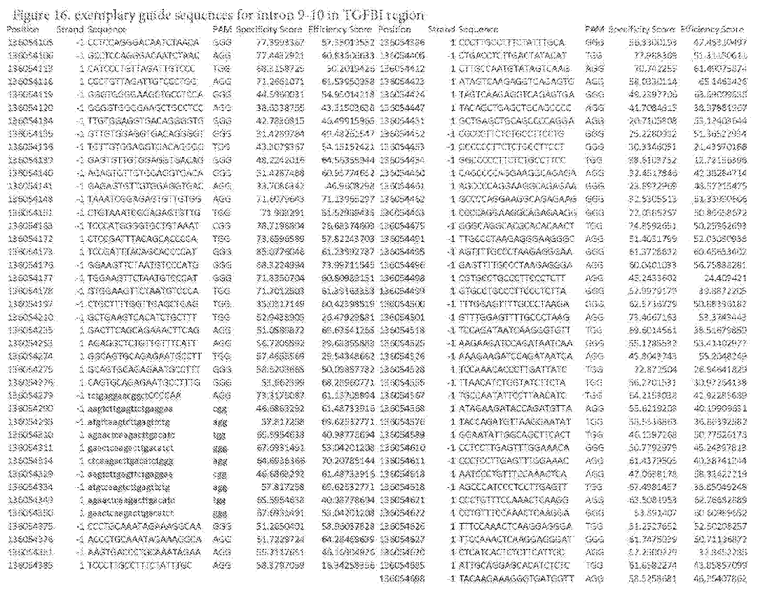

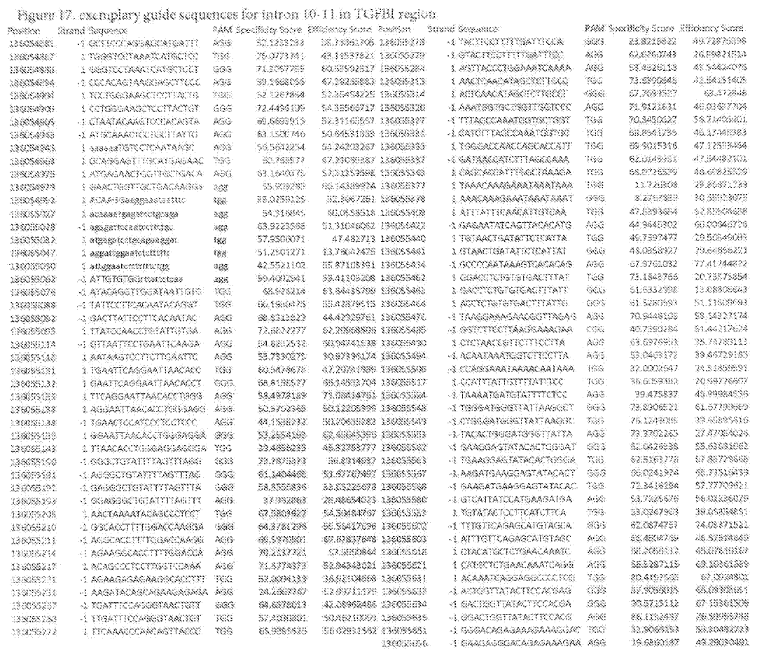

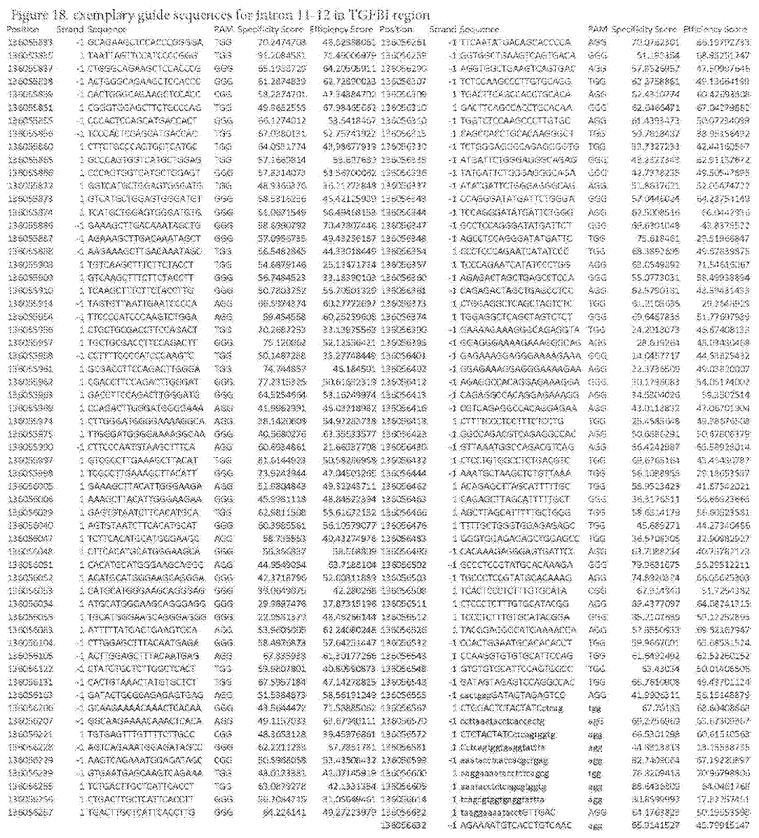

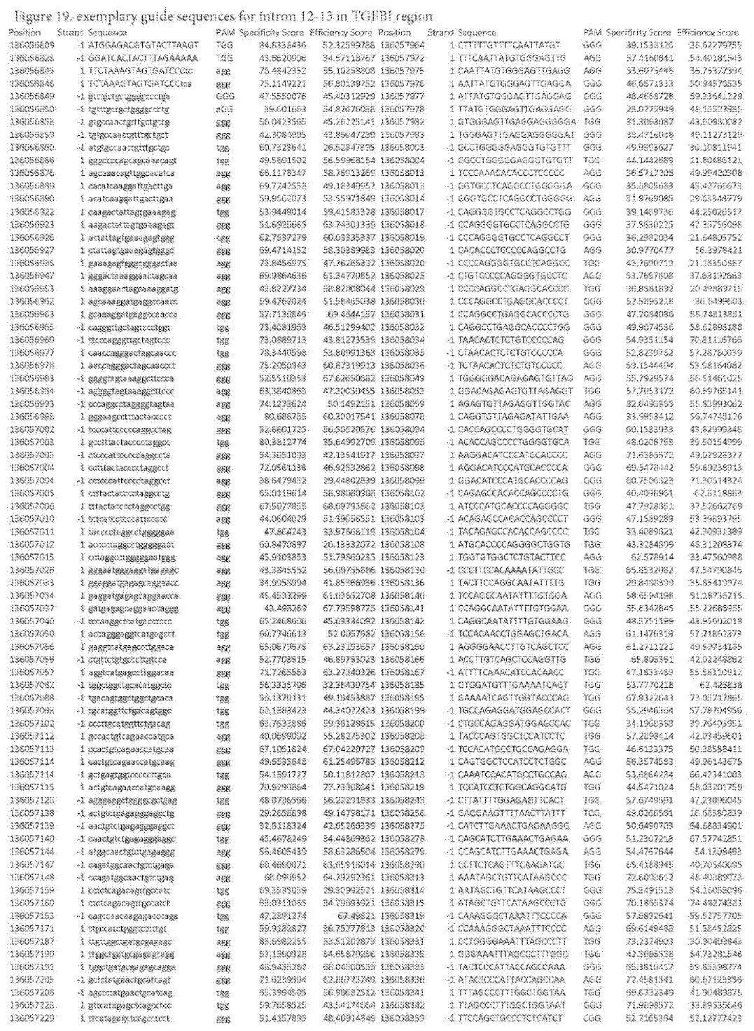

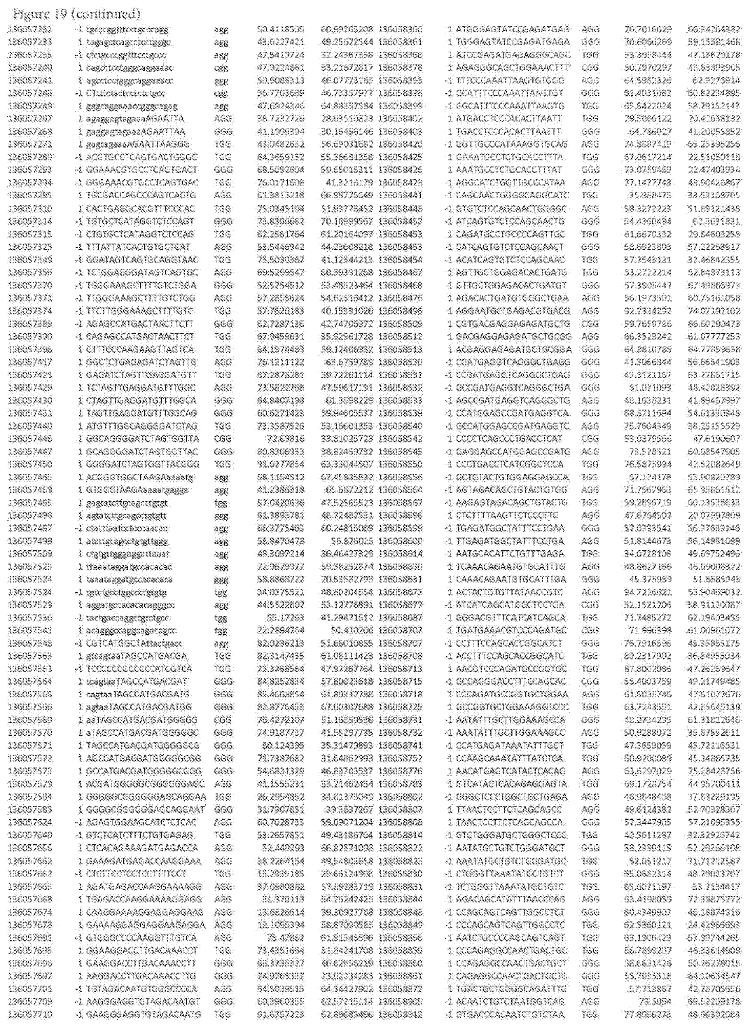

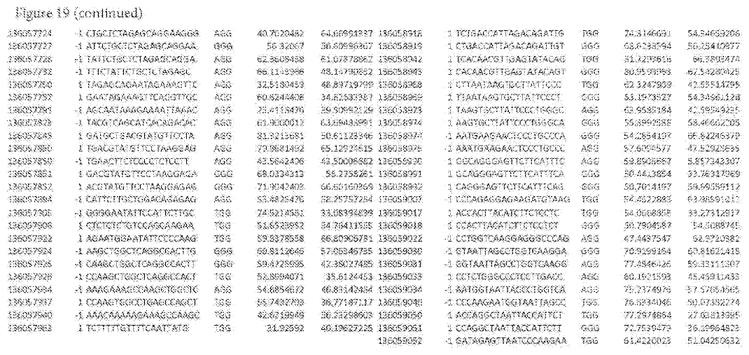

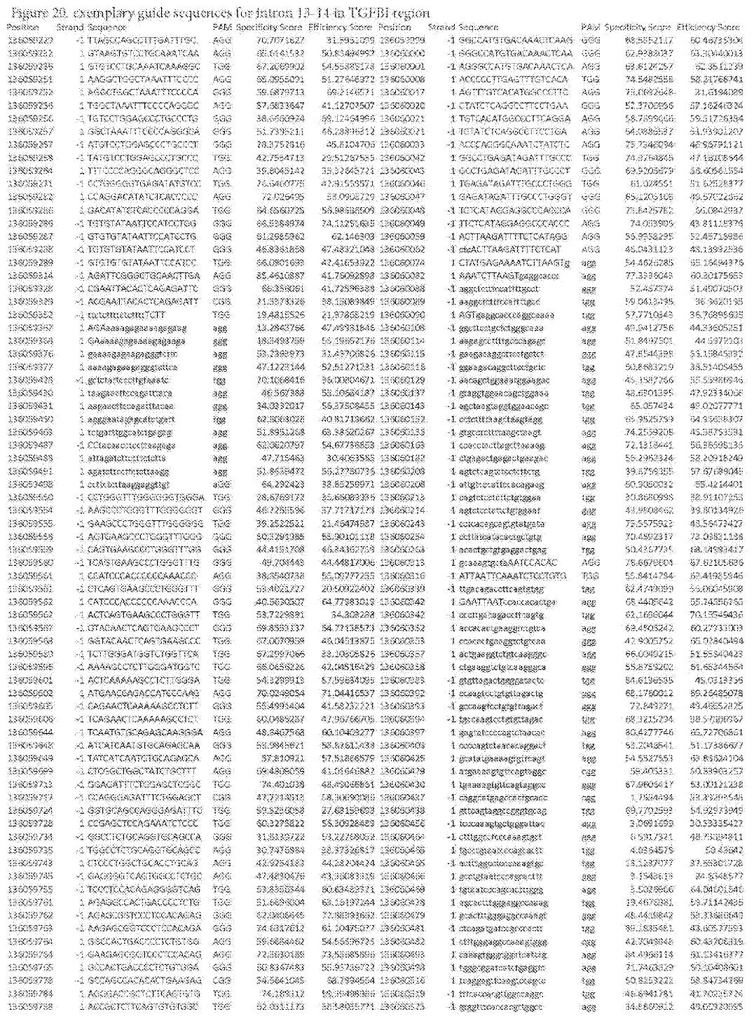

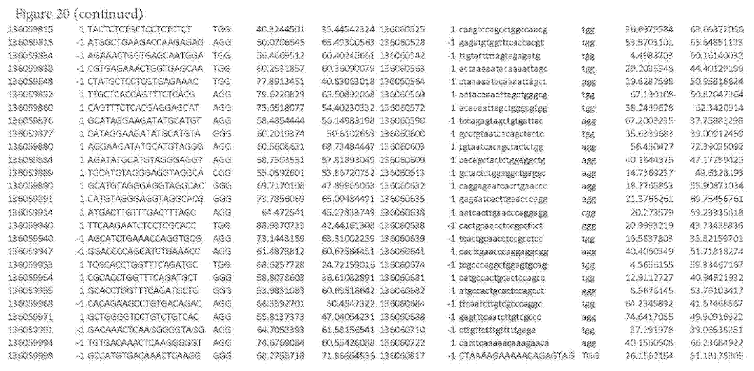

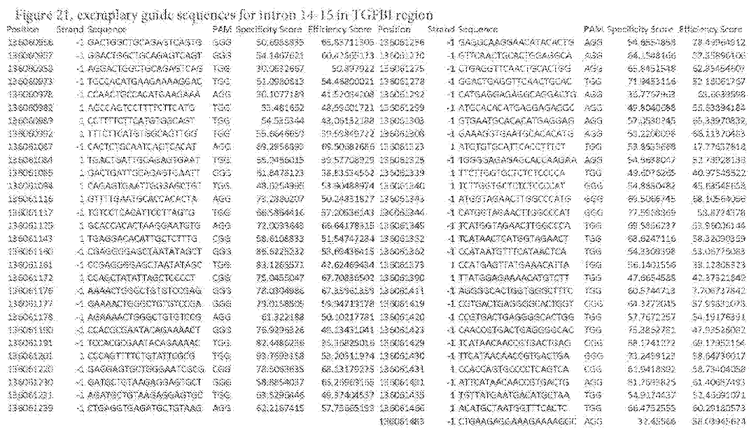

The term “crRNA” may refer to a guide sequence that may be a part of an sgRNA in an CRISPR/Cas9 system. In some embodiments, at least one of the first and second crRNA sequences described herein comprises a nucleotide sequence selected from the group consisting of sequences listed in FIGS. 8-23; and/or at least one of the first and second crRNA sequences comprises a nucleotide sequence selected from the group consisting of sequences listed in Table 2. The term, “sgRNA” refers to a single guide RNA containing (i) a guide sequence (crRNA sequence) and (ii) a Cas9 nuclease-recruiting sequence (tracrRNA). The crRNA sequence may be a sequence that is homologous to a region in your gene of interest and may direct Cas9 nuclease activity. The crRNA sequence and tracrRNA sequence may not naturally occur together. The sgRNA may be delivered as RNA or by transforming with a plasmid with the sgRNA-coding sequence (sgRNA gene) under a promoter. The tracrRNA sequence may be any sequence for tracrRNA for CRISPR/Cas9 system known in the art.

In some embodiments, the crRNA hybridizes to at least a part of a target sequence (e.g., target genome sequence), and the crRNA may have a complementary sequence to the target sequence. In some embodiments, the target sequence herein is a first target sequence that hybridizes to a second target sequence adjacent to a PAM site described herein. In some embodiments, the crRNA may comprise the first target sequence or the second target sequence. In additional embodiments, the first and second target sequences are located in introns of a target gene. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary), “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least about 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. In additional embodiments, the crRNA or the guide sequence is about 17, 18, 19, 20, 21, 22, 23 or 24 nucleotide long. As used herein, the term “about” may refer to a range of values that are similar to the stated reference value. In certain embodiments, the term “about” refers to a range of values that fall within 15, 10, 9, 8, 7, 6, 5, 4, 3, 2, 1 percent or less of the stated reference value.

In one aspect, the present disclosure is related to methods of preventing, ameliorating, or treating a disease associated with a gene mutation or single-nucleotide polymorphism (SNP) in a subject, comprising administering to the subject an engineered CRISPR/Cas9 system comprising at least one vector comprising (i) a nucleotide molecule encoding Cas9 nuclease; (ii) a first sgRNA comprising a first CRISPR targeting RNA (crRNA) sequence that hybridizes to a nucleotide sequence complementary to a first target sequence, the first target sequence being adjacent to the 5′-end of a first protospacer adjacent motif (PAM) in a first intron at 3′-end side of an exon comprising a disease-causing mutation or SNP in cis, wherein the first target sequence or the first PAM comprises a first ancestral variation or SNP site; and (iii) a second sgRNA comprising a second crRNA sequence that hybridizes to a nucleotide sequence complementary to a second target sequence, the second target sequence being adjacent to the 5′-end of a second PAM in a second intron at 5′-end side of the exon comprising the disease-causing mutation or SNP in cis, wherein the second target sequence or the second PAM comprises a second ancestral variation or SNP site, wherein at least one vector does not have a nucleotide molecule encoding Cas9 nuclease and a crRNA sequence that naturally occur together. In another aspect, the present disclosure is related to methods of preventing, ameliorating, or treating a disease associated with a gene mutation or single-nucleotide polymorphism (SNP) in a subject comprising altering expression of the gene product of the subject by the methods described above, wherein the gene comprises a mutant or SNP mutant sequence. In some embodiments, the disease is associated with the SNP; the first target sequence or the first PAM comprises the first ancestral SNP site; and/or the second target sequence or the second PAM comprises the second ancestral SNP site. In additional embodiments, the target sequence comprises a plurality of mutation or SNP sites. In some embodiments, the subject is human. In some embodiments, the first crRNA sequence hybridizes to the nucleotide sequence so that the Cas9 nuclease cleaves at a first cleaving site that is adjacent to the first ancestral variation or SNP site; and/or the second crRNA sequence hybridizes to the nucleotide sequence so that the Cas9 nuclease cleaves at a second cleaving site that is adjacent to the second ancestral variation or SNP site. In additional embodiments, the first crRNA sequence hybridizes to the nucleotide sequence so that the Cas9 nuclease cleaves only at the first cleaving site; and/or the second crRNA sequence hybridizes to the nucleotide sequence so that the Cas9 nuclease cleaves only at the second cleaving site.

As described herein, being “in cis” with the disease-causing mutation or SNP refers to being on the same molecule of DNA or chromosome as the disease-causing mutation, and being “in trans” with the disease-causing mutation or SNP refers to being on a different molecule of DNA or chromosome as the disease-causing mutation or SNP. In some embodiments, the first crRNA sequence hybridizes to the nucleotide sequence complementary to the first target sequence in trans with the disease-causing mutation or SNP, said first target sequence not being adjacent to the 5′-end of a PAM; and/or the second crRNA sequence hybridizes to the nucleotide sequence complementary to the second target sequence in trans with the disease-causing mutation or SNP, said second target sequence not being adjacent to the 5′-end of a PAM. In the absence of the PAM adjacent to the first and/or second target sequences, the first and/or the second target sequences in trans with the disease-causing mutation or SNP may remain intact without any cleavage (e.g., the Cas9 nuclease does not cleave the first and/or the second target sequences in trans with the disease-causing mutation or SNP). This approach may permit expression of a gene that is in trans with the disease-causing mutation or SNP and does not include a disease-causing mutation or SNP. This approach may also reduce or eliminate any adverse impacts associated with knocking out both the gene that includes the disease-causing mutation or SNP and the gene that does not include the disease-causing mutation or SNP in a subject. In additional embodiments, the first crRNA sequence hybridizes to the nucleotide sequence complementary to the first target sequence in trans with the disease-causing mutation or SNP, said first target sequence not being adjacent to the 5′-end of a PAM; and the second crRNA sequence hybridizes to the nucleotide sequence complementary to the second target sequence in trans with the disease-causing mutation or SNP, said second target sequence being adjacent to the 5′-end of a PAM. In the absence of the PAM adjacent to the first target sequence, the first target sequence in trans with the disease-causing mutation or SNP may remain intact without any cleavage while the second target sequence in trans with the disease-causing mutation or SNP may be cleaved (e.g., the Cas9 nuclease cleaves the first target sequence in trans with the disease-causing mutation or SNP but does not cleave the second target sequence in trans with the disease-causing mutation or SNP). In further embodiments, the first crRNA sequence hybridizes to the nucleotide sequence complementary to the first target sequence in trans with the disease-causing mutation or SNP, said first target sequence being adjacent to the 5′-end of a PAM; and the second crRNA sequence hybridizes to the nucleotide sequence complementary to the second target sequence in trans with the disease-causing mutation or SNP, said second target sequence not being adjacent to the 5′-end of a PAM. In the absence of the PAM adjacent to the second target sequence, the second target sequence in trans with the disease-causing mutation or SNP may remain intact without any cleavage while the first target sequence in trans with the disease-causing mutation or SNP is cleaved (e.g., the Cas9 nuclease cleaves the second target sequence in trans with the disease-causing mutation or SNP but does not cleave the first target sequence in trans with the disease-causing mutation or SNP). Said “nucleotide sequence complementary to the first target sequence in trans with the disease-causing mutation or SNP” herein has the identical nucleotide sequence as the nucleotide sequence complementary to the first target sequence in cis with the disease-causing mutation or SNP. Said “nucleotide sequence complementary to the first target sequence in trans with the disease-causing mutation or SNP” and said “the first target sequence in trans with the disease-causing mutation or SNP,” however, may be located on a different molecule of DNA or chromosome where the same disease-causing mutation or SNP is absent (thus are in trans with the disease-causing mutation or SNP). Similarly, said “nucleotide sequence complementary to the second target sequence in trans with the disease-causing mutation or SNP” herein has the identical nucleotide sequence as the nucleotide sequence complementary to the second target sequence in cis with the disease-causing mutation or SNP. Said “nucleotide sequence complementary to the second target sequence in trans with the disease-causing mutation or SNP” and said “the second target sequence in trans with the disease-causing mutation or SNP,” however, may be located on a different molecule of DNA or chromosome where the disease-causing mutation or SNP is absent (thus are in trans with the disease-causing mutation or SNP).

In some embodiments, the engineered CRISPR/Cas9 system described herein may comprise at least one vector comprising (i) a nucleotide molecule encoding Cas9 nuclease described herein, and (ii) a plurality of sgRNA targeting intronic sites surrounding one or more exons containing a disease-associate mutation or SNP of interest as described herein. The sgRNA may comprise a target sequence adjacent to the 5′-end of a protospacer adjacent motif (PAM), and/or hybridize to a first target sequence complementary to a second target sequence adjacent to the 5′ end of the PAM. The target sequence or the PAM may comprise the ancestral variation or SNP in an intronic site. In additional embodiments, the ancestral variation or SNP in the intronic site does not cause a disease. In some embodiments, sgRNA may comprise a target sequence adjacent to a PAM site located in the flanking intron that is common to both wild-type and mutant alleles in tandem with a sgRNA adjacent to a PAM site that is specific to the mutant allele. In some embodiments, the Cas9 nuclease and the sgRNA do not naturally occur together. The sequence of this PAM site is specific to the Cas9 nuclease being used. In additional embodiments, the PAM comprises the mutation or SNP site. In yet additional embodiments, the PAM consists of a PAM selected from the group consisting of NGG and NNGRRT, wherein N is any of A, T, G, and C, and R is A or G.

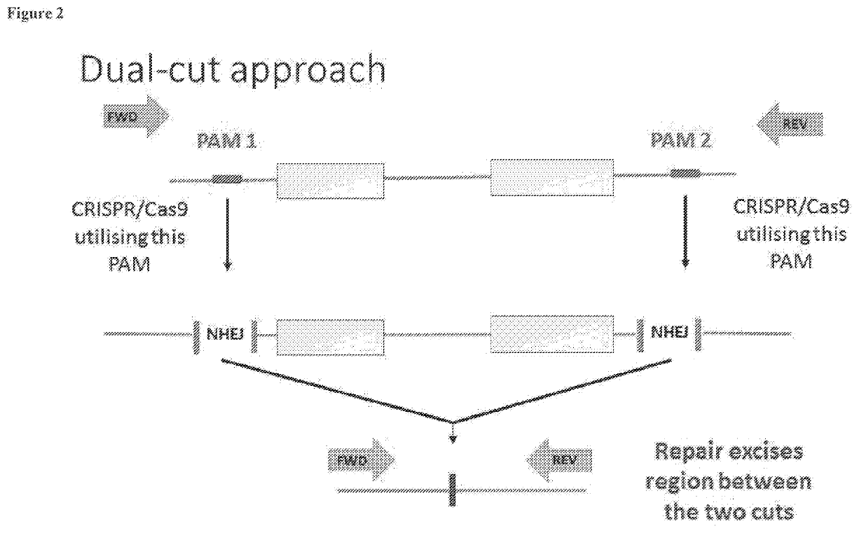

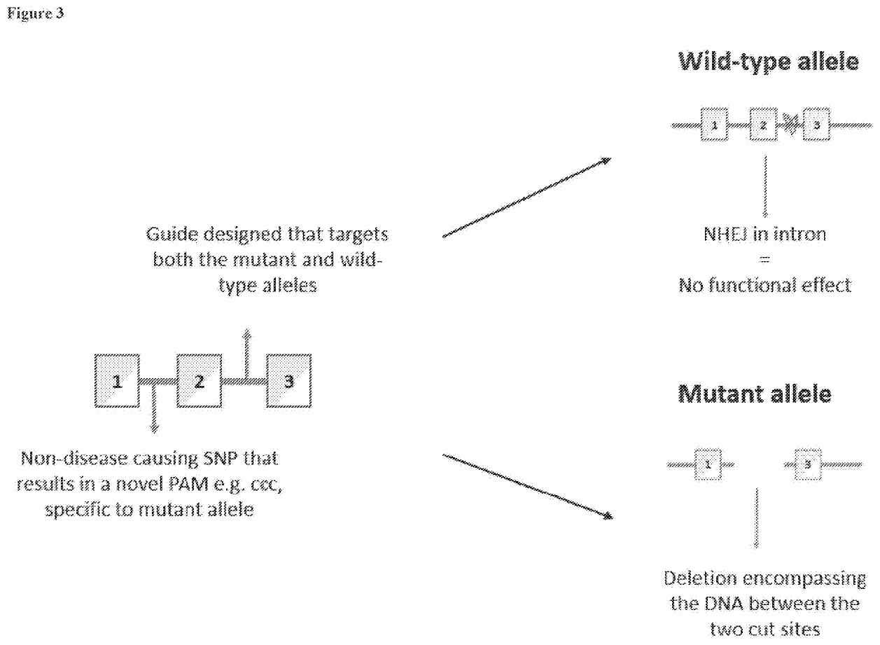

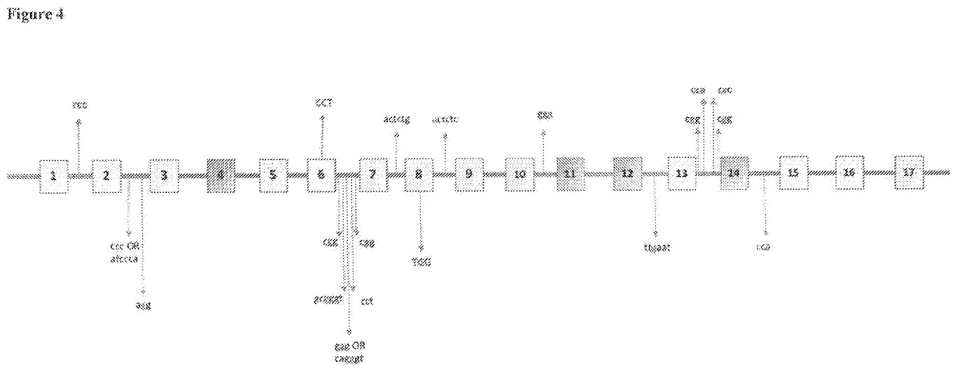

In some embodiments, the disease-causing mutation or SNP is in an exon of a gene associated with the disease, and the first and second PAMs are in different introns surrounding one or more exons containing the disease-causing mutation or SNP. This may be called a dual-cut approach. As shown in FIGS. 2 and 3, first and second CRISPR targeting RNA (crRNA) sequences hybridize to nucleotide sequences complementary to first and second target sequences, the first target sequence being adjacent to the 5′-end of a first protospacer adjacent motif (PAM) in a first intron at 3′-end side of an exon comprising a disease-causing mutation or SNP in cis, and the second target sequence being adjacent to the 5′-end of a first protospacer adjacent motif (PAM) in a second intron at 5′-end side of the exon comprising the disease-causing mutation or SNP in cis. Thus, the first and second PAMs are located on opposite sides of one or more exons containing the disease-causing mutation or SNP. As used herein, an “intron” means a section of DNA occurring between two adjacent exons within a gene which is removed during pre-mRNA splicing and does not code for any amino acids constituting the gene product. An “intronic site” is a site within an intron. An “exon” means a section of DNA occurring in a gene which codes for one or more amino acids in the gene product. For example, the constitutively spliced exon known so far has 6 nucleotides or more, and the alternatively spliced exon has 3 nucleotides or more, which is equivalent to 1 or 2 amino acids or more depending on the frame that the mRNA is read in. An “exonic site” is a site within an exon.

In some embodiments, the first PAM comprises the first mutation or SNP site and/or the second PAM comprises the second mutation or SNP site. In some embodiments, the first crRNA sequence comprises the first target sequence, and the second crRNA sequence comprises the second target sequence. In further embodiments, each of the first crRNA sequence and the second crRNA sequence may independent be from 14, 15, 16, 17, 18, 19, 20, 21, 22, 23, 24 or 25 to 20, 21, 22, 23, 24, 25, 26, 27, 28, 29 or 30 nucleotide long.

In some embodiments, the methods described herein further comprise identifying targetable mutations or SNPs on either side of disease-causing mutation or SNP to silence the disease-causing mutation or SNP. In some embodiments, a block of DNA is identified in a phased sequencing experiment. In some embodiments, the mutation or SNP of interest is not a suitable substrate for the CRISPR/Cas9 system, and identifying mutations or SNPs on both side of the disease-causing mutations or SNP that are suitable for CRISPR/Cas9 cleavage allows removal of a segment of DNA that includes the disease-causing mutations or SNP. In some embodiments, the read length may be increased so as to gain longer contiguous reads and a haplotype phased genome by using a technology described in Weisenfeld N I, Kumar V, Shah P, Church D M, Jaffe D B. Direct determination of diploid genome sequences. Genome research. 2017; 27(5):757-767, which is herein incorporated by reference in its entirety.

In some embodiments, the methods described herein further comprises, prior to administering to the subject the engineered CRISPR/Cas9 system, obtaining genomic or sequence information of the subject; and selecting the first crRNA sequence and/or the second crRNA sequence based on the genomic or sequence information of the subject. In additional embodiments, the genomic or sequence information of the subject includes whole or partial genome sequence information of the subject.

The human genome is diploid by nature; every chromosome with the exception of the X and Y chromosomes in males is inherited as a pair, one from the male and one from the female parent. When seeking stretches of contiguous DNA sequence larger than a few thousand base pairs, a determination of inheritance is crucial to understand from which parent these blocks of DNA originate. Longer read sequencing technologies have been utilized in attempts to produce a haplotype-resolved genome sequences, i.e. haplotype phasing. Thus, when investigating the genomic sequence of a particular stretch of DNA longer than 50 kbps, a haplotype phased sequence analysis may be utilized to determine which of the paired chromosomes carries the sequence of interest. Longer phased sequencing reads may be employed to determine whether the SNP of interest would be suitable as a target for the CRISPR/Cas9 gene editing system described herein.

In some embodiments, the selected first crRNA sequence is configured to cause cleaving at a first cleaving site, within genome of the subject, that is adjacent to the first ancestral variation or SNP site; and/or the selected second crRNA sequence is configured to cause cleaving at a second cleaving site, within the genome of the subject, that is adjacent to the second ancestral variation or SNP site. In additional embodiments, the selected first crRNA sequence is configured to cause cleaving only at the first cleaving site; and/or the selected second crRNA sequence is configured to cause cleaving only at the second cleaving site. In some embodiments, the selected first crRNA sequence hybridizes to the nucleotide sequence (in trans) complementary to the first target sequence in trans with the disease-causing mutation or SNP, said first target sequence not being adjacent to the 5′-end of a PAM; and/or the selected second crRNA sequence hybridizes to the nucleotide sequence (in trans) complementary to the second target sequence in trans with the disease-causing mutation or SNP, said second target sequence not being adjacent to the 5′-end of a PAM. In additional embodiments, the selected first crRNA sequence hybridizes to the nucleotide sequence (in trans) complementary to the first target sequence in trans with the disease-causing mutation or SNP, said first target sequence not being adjacent to the 5′-end of a PAM; and the selected second crRNA sequence hybridizes to the nucleotide sequence (in trans) complementary to the second target sequence in trans with the disease-causing mutation or SNP, said second target sequence being adjacent to the 5′-end of a PAM. In further embodiments, the selected first crRNA sequence hybridizes to the nucleotide sequence (in trans) complementary to the first target sequence in trans with the disease-causing mutation or SNP, said first target sequence being adjacent to the 5′-end of a PAM; and the selected second crRNA sequence hybridizes to the nucleotide sequence (in trans) complementary to the second target sequence in trans with the disease-causing mutation or SNP, said second target sequence not being adjacent to the 5′-end of a PAM.

In some embodiments, selecting the first crRNA sequence includes selecting a crRNA sequence that corresponds to the first target sequence in trans, said first target sequence in trans not being adjacent to the 5′-end of a PAM, and/or selecting the second crRNA sequence includes selecting a crRNA sequence that corresponds to the second target sequence in trans, said second target sequence in trans not being adjacent to the 5′-end of a PAM. In some embodiments, selecting the first crRNA sequence includes selecting a crRNA sequence that corresponds to the first target sequence in trans, said first target sequence in trans not being adjacent to the 5′-end of a PAM, and selecting the second crRNA sequence includes selecting a crRNA sequence that corresponds to the second target sequence in trans, said second target sequence in trans being adjacent to the 5′-end of a PAM. In some embodiments, selecting the first crRNA sequence includes selecting a crRNA sequence that corresponds to the first target sequence in trans, said first target sequence in trans being adjacent to the 5′-end of a PAM, and selecting the second crRNA sequence includes selecting a crRNA sequence that corresponds to the second target sequence in trans, said second target sequence in trans not being adjacent to the 5′-end of a PAM.

In some embodiments, the subjects that can be treated with the methods described herein include, but are not limited to, mammalian subjects such as a mouse, rat, dog, baboon, pig or human. In some embodiments, the subject is a human. The methods can be used to treat subjects at least 1 year, 2 years, 3 years, 5 years, 10 years, 15 years, 20 years, 25 years, 30 years, 35 years, 40 years, 45 years, 50 years, 55 years, 60 years, 65 years, 70 years, 75 years, 80 years, 85 years, 90 years, 95 years or 100 years of age. In some embodiments, the subject is treated for at least one, two, three, or four diseases. For example, a single or multiple crRNA or sgRNA may be designed to alter or delete nucleotides at more than 2, 3, 4, 5, 6, 7, 8, 9 or 10 and/or fewer than 20, 10, 9, 8, 7, 6, 5, 4 or 3 ancestral variation or SNP sites.

In some embodiments, the methods of preventing, ameliorating, or treating the disease in a subject may comprise administering to the subject an effective amount of the engineered CRISPR/Cas9 system described herein. The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

In some embodiments, the administering comprises injecting the engineered CRISPR/Cas9 system into the subject. In additional embodiments, the administering comprises introducing the engineered CRISPR/Cas9 system into a cell containing and expressing a DNA molecule having the target sequence as described below.

In some embodiments, the methods of treating the disease provide a positive therapeutic response with respect to a disease or condition. By “positive therapeutic response” is intended an improvement in the disease or condition, and/or an improvement in the symptoms associated with the disease or condition. The therapeutic effects of the subject methods of treatment can be assessed using any suitable method. In some embodiments, the subject methods reduce the amount of a disease-associate protein deposition in the subject by at least 5%, 10%, 15%, 20%, 25%, 30%, 35%, 40%, 45%, 50%, 55%, 60%, 65%, 70%, 75%, 80%, 85%, 90%, 95%, or 99% as compared to the subject prior to undergoing treatment.

In another aspect, the present disclosure is related to engineered Clustered Regularly Interspaced Short Palindromic Repeats (CRISPR)/CRISPR associate protein 9 (Cas9) systems for preventing, ameliorating or treating corneal dystrophies. The CRISPR/Cas9 may comprise at least one vector comprising a nucleotide molecule encoding Cas9 nuclease and the sgRNAs and/or crRNAs as described herein. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. In some embodiments, the Cas9 nuclease and the sgRNA/crRNA do not naturally occur together.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as “crRNA” herein, or a “spacer” in the context of an endogenous CRISPR system), and/or other sequences and transcripts from a CRISPR locus. As described above, sgRNA is a combination of at least tracrRNA and crRNA. In some embodiments, one or more elements of a CRISPR system are derived from a type II CRISPR system. In some embodiments, one or more elements of a CRISPR system are derived from a particular organism comprising an endogenous CRISPR system, such as Streptococcus pyogenes or Staphylococcus aureus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” may refer to a sequence to which a guide sequence is designed to have complementarity, where hybridization between a target sequence and a guide sequence promotes the formation of a CRISPR complex, the “target sequence” may refer to a sequence adjacent to a PAM site, which the guide sequence comprises. Full complementarity is not necessarily required, provided there is sufficient complementarity to cause hybridization and promote formation of a CRISPR complex. In this disclosure, “target site” refers to a site of the target sequence including both the target sequence and its complementary sequence, for example, in double stranded nucleotides. In some embodiments, the target site described herein may mean a first target sequence hybridizing to sgRNA or crRNA of CRISPR/Cas9 system, and/or a second target sequence adjacent to the 5′-end of a PAM. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast.

The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells.

In some embodiments, at least one vector of the engineered CRISPR/Cas9 system described herein further comprises (a) a first regulatory element operably linked to the sgRNA that hybridizes with the target sequence described herein, and (b) a second regulatory element operably linked to the nucleotide molecule encoding Cas9 nuclease, wherein components (a) and (b) are located on a same vector or different vectors of the system, the sgRNA targets the target sequence, and the Cas9 nuclease cleaves the DNA molecule. The target sequence may be a nucleotide sequence complementary to from 16 to 25 nucleotides adjacent to the 5′ end of a PAM. Being “adjacent” herein means being within 2 or 3 nucleotides of the site of reference, including being “immediately adjacent,” which means that there is no intervening nucleotides between the immediately adjacent nucleotide sequences and the immediate adjacent nucleotide sequences are within 1 nucleotide of each other. In additional embodiments, the cell is a eukaryotic cell, or a mammalian or human cell, and the regulatory elements are eukaryotic regulators. In further embodiments, the cell is a stem cell described herein. In some embodiments, the Cas9 nuclease is codon-optimized for expression in a eukaryotic cell.

In some embodiments, the first regulatory element is a polymerase III promoter. In some embodiments, the second regulatory element is a polymerase II promoter. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the Cas9 nuclease provided herein may be an inducible Cas9 nuclease that is optimized for expression in a temporal or cell-type dependent manner. The first regulatory element may be an inducible promoter that can be linked to the Cas9 nuclease include, but are not limited to, tetracycline-inducible promoters, metallothionein promoters; tetracycline-inducible promoters, methionine-inducible promoters (e.g., MET25, MET3 promoters); and galactose-inducible promoters (GAL1, GAL7 and GAL10 promoters). Other suitable promoters include the ADH1 and ADH2 alcohol dehydrogenase promoters (repressed in glucose, induced when glucose is exhausted and ethanol is made), the CUP1 metallothionein promoter (induced in the presence of Cu²⁺, Zn²⁺), the PHO5 promoter, the CYC1 promoter, the HIS3 promoter, the PGK promoter, the GAPDH promoter, the ADC1 promoter, the TRP1 promoter, the URA3 promoter, the LEU2 promoter, the ENO promoter, the TP1 promoter, and the AOX1 promoter.

It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Exemplary CRISPR/Cas9 systems, sgRNA, crRNA and tracrRNA, and their manufacturing process and use are disclosed in U.S. Pat. No. 8,697,359, U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, 20140080216, and WO2016049024, all of which are incorporated herein by their entirety.

In some embodiments, the Cas9 nucleases described herein are known; for example, the amino acid sequence of S. pyogenes Cas9 protein may be found in the SwissProt database under accession number Q99ZW2. The Cas9 nuclease may be a Cas9 homolog or ortholog. Mutant Cas9 nucleases that exhibit improved specificity may also be used (see, e.g., Ann Ran et al. Cell 154(6) 1380-89 (2013), which is herein incorporated by reference in its entirety for all purposes and particularly for all teachings relating to mutant Cas9 nucleases with improved specificity for target nucleic acids). The nucleic acid manipulation reagents can also include a deactivated Cas9 nuclease (dCas9). Deactivated Cas9 binding to nucleic acid elements alone may repress transcription by sterically hindering RNA polymerase machinery. Further, deactivated Cas may be used as a homing device for other proteins (e.g., transcriptional repressor, activators and recruitment domains) that affect gene expression at the target site without introducing irreversible mutations to the target nucleic acid. For example, dCas9 can be fused to transcription repressor domains such as KRAB or SID effectors to promote epigenetic silencing at a target site. Cas9 can also be converted into a synthetic transcriptional activator by fusion to VP16/VP64 or p64 activation domains. In some instances, a mutant Type II nuclease, referred to as an enhanced Cas9 (eCa9) nuclease, is used in place of the wild-type Cas9 nuclease. The enhanced Cas9 has been rationally engineered to improve specificity by weakening non-target binding. This has been achieved by neutralizing positively charged residues within the non-target strand groove (Slaymaker et al., 2016).

In some embodiments, the Cas9 nucleases direct cleavage of one or both strands at the location of a target sequence, such as within the target sequence and/or within the complement of the target sequence. In some embodiments, the Cas9 nucleases directs cleavage of one or both strands within about 1, 2, 3, 4, 5, 6, 7, 8, 9, 10, 15, 20, 25, 50, 100, 200, 500, or more base pairs from the first or last nucleotide of a target sequence.

Following directed DNA cleavage by the Cas9 nuclease, there are two modes of DNA repair available to the cell: homology directed repair (HDR) and non-homologous end joining (NHEJ). While seamless correction of the mutation by HDR following Cas9 cleavage close to the mutation site is attractive, the efficiency of this method means that it could only be used for in vitro/ex vivo modification of stem cells or induced pluripotent stem cells (iPSC) with an additional step to select those cells in which repair had taken place and purify those modified cells only. HDR does not occur at a high frequency in cells.

In some embodiments, the first and/or second PAMs and the Cas9 nuclease described herein are from Streptococcus or Staphylococcus. In additional embodiments, the first and second PAMs are both from Streptococcus or Staphylococcus. In additional embodiments, the Cas9 nuclease is from Streptococcus. In yet additional embodiments, the Cas9 nuclease is from Streptococcus pyogenes, Streptococcus dysgalactiae, Streptococcus canis, Streptococcus equi, Streptococcus iniae, Streptococcus phocae, Streptococcus pseudoporcinus, Streptococcus oralis, Streptococcus pseudoporcinus, Streptococcus infantarius, Streptococcus mutans, Streptococcus agalactiae, Streptococcus caballi, Streptococcus equinus, Streptococcus sp. oral taxon, Streptococcus mitis, Streptococcus gallolyticus, Streptococcus gordonii, or Streptococcus pasteurianus, or variants thereof. Such variants may include D10A Nickase, Spy Cas9-HF1 as described in Kleinstiver et al, 2016 Nature, 529, 490-495, or Spy eCas9 as described in Slaymaker et al., 2016 Science, 351(6268), 84-88. In additional embodiments, the Cas9 nuclease is from Staphylococcus. In yet additional embodiments, the Cas9 nuclease is from Staphylococcus aureus, S. simiae, S. auricularis, S. carnosus, S. condiments, S. massiliensis, S. piscifermentans, S. simulans, S. capitis, S. caprae, S. epidermidis, S. saccharolyticus, S. devriesei, S. haemolyticus, S. hominis, S. agnetis, S. chromogenes, S. felis, S. delphini, S. hyicus, S. intermedius, S. lutrae, S. microti, S. muscae, S. pseudintermedius, S. rostri, S. schleiferi, S. lugdunensis, S. arlettae, S. cohnii, S. equorum, S. gallinarum, S. kloosii, S. leei, S. nepalensis, S. saprophyticus, S. succinus, S. xylosus, S. fleurettii, S. lentus, S. sciuri, S. stepanovicii, S. vitulinus, S. simulans, S. pasteuri, S. warneri, or variants thereof.

In further embodiments, the Cas9 nuclease excludes Cas9 nuclease from Streptococcus pyogenes.

In additional embodiments, the Cas9 nuclease comprises an amino acid sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with an amino acid sequence selected from the group consisting of SEQ ID NO: 4 or 8. In additional embodiments, the nucleotide molecule encoding Cas9 nuclease comprises a nucleotide sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a nucleotide sequence selected from the group consisting of SEQ ID NO: 3 or 7. In yet additional embodiments, Cas9 sgRNA sequence may comprises a sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO: 1 or 5. An exemplary tracrRNA or sgRNA scaffold sequence may comprise a sequence having at least about 60, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with SEQ ID NO: 2 or 6.

In some embodiments, the Cas9 nuclease is an enhanced Cas9 nuclease that has one or more mutations improving specificity of the Cas9 nuclease. In additional embodiments, the enhanced Cas9 nuclease is from a Cas9 nuclease from Streptococcus pyogenes having one or more mutations neutralizing a positively charged groove, positioned between the HNH, RuvC, and PAM-interacting domains in the Cas9 nuclease. In yet additional embodiments, the Cas9 nuclease comprises an amino acid sequence having at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a mutant amino acid sequence of a Cas9 nuclease from Streptococcus pyogenes (e.g., SEQ ID NO: 4) with one or more mutations selected from the group consisting of (i) K855A, (ii) K810A, K1003A and R1060A, and (iii) K848A, K1003A and R1060A. In yet further embodiments, the nucleotide molecule encoding Cas9 nuclease comprises a nucleotide sequence having at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with a nucleotide sequence encoding the mutant amino acid sequence.

In some embodiments, the CRISPR/Cas9 system and the methods using the CRISPR/Cas9 system described herein alter a DNA sequence by the NHEJ. In additional embodiments, the CRISPR/Cas9 system or the vector described herein does not include a repair nucleotide molecule. In some embodiments, the methods described herein alter a DNA sequence by the HDR. In some embodiments, the CRISPR/Cas9 system or the vector described herein may further comprise a repair nucleotide molecule. The target polynucleotide cleaved by the Cas9 nuclease may be repaired by homologous recombination with the repair nucleotide molecule, which is an exogenous template polynucleotide. This repair may result in a mutation comprising an insertion, deletion, or substitution of one or more nucleotides of said target polynucleotide. The repair nucleotide molecule introduces a specific allele (e.g., a wild-type allele) into the genome of one or more cells of the plurality of stem cells upon repair of a Type II nuclease induced DSB through the HDR pathway. In some embodiments, the repair nucleotide molecule is a single stranded DNA (ssDNA). In other embodiments, the repair nucleotide molecule is introduced into the cell as a plasmid vector. In some embodiments, the repair nucleotide molecule is 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 105, 105 to 110, 110 to 115, 115 to 120, 120 to 125, 125 to 130, 130 to 135, 135 to 140, 140 to 145, 145 to 150, 150 to 155, 155 to 160, 160 to 165, 165 to 170, 170 to 175, 175 to 180, 180 to 185, 185 to 190, 190 to 195, or 195 to 200 nucleotides in length. In some embodiments, the repair nucleotide molecule is 200 to 300, 300, to 400, 400 to 500, 500 to 600, 600 to 700, 700 to 800, 800 to 900, 900 to 1,000 nucleotides in length. In other embodiments, the repair nucleotide molecule is 1,000 to 2,000, 2,000 to 3,000, 3,000 to 4,000, 4,000 to 5,000, 5,000 to 6,000, 6,000 to 7,000, 7,000 to 8,000, 8,000 to 9,000, or 9,000 to 10,000 nucleotides in length.

The repair nucleotide molecule may further include a label for identification and sorting of cells described herein containing the specific mutation. Exemplary labels that can be included with the repair nucleotide molecule include fluorescent labels and nucleic acid barcodes that are identifiable by length or sequence.

In additional embodiments, the CRISPR/Cas9 system or the vector described herein may include at least one nuclear localization signal (NLS). In additional embodiments, the sgRNA and the Cas9 nuclease are included on the same vector or on different vectors.

As used herein, a “corneal dystrophy” refers to any one of a group of hereditary disorders in the outer layer of the eye (cornea). For example, the corneal dystrophy may be characterized by bilateral abnormal deposition of substances in the cornea. Corneal dystrophies include, but are not limited to the following four IC3D categories of corneal dystrophies (see, e.g., Weiss et al., Cornea 34(2): 117-59 (2015)): epithelial and sub-epithelial dystrophies, epithelial-stromal TGFβI dystrophies, stromal dystrophies and endothelial dystrophies. In some embodiments, the corneal dystrophy is selected from the group consisting of Epithelial basement membrane dystrophy (EBMD), Meesmann corneal dystrophy (MECD), Thiel-Behnke corneal dystrophy (TBCD), Lattice corneal dystrophy (LCD), Granular corneal dystrophy (GCD), and Schnyder corneal dystrophy (SCD). In additional embodiments, the corneal dystrophy is caused by one or more mutations, including SNP, is located in a gene selected from the group consisting of Transforming growth factor, beta-induced (TGFBI), keratin 3 (KRT3), keratin 12 (KRT12), GSN, and UbiA prenyltransferase domain containing 1 (UBIAD1). In further embodiments, the mutation or SNP site results in encoding a mutant amino acid in a mutant protein as shown herein. In further embodiments, a mutant sequence comprising the mutation or SNP site encodes a mutant protein selected from the group consisting of (i) mutant TGFBI proteins comprising a mutation corresponding to Leu509Arg, Arg666Ser, Gly623Asp, Arg555Gln, Arg124Cys, Val505Asp, Ile522Asn, Leu569Arg, His572Arg, Arg496Trp, Pro501Thr, Arg514Pro, Phe515Leu, Leu518Pro, Leu518Arg, Leu527Arg, Thr538Pro, Thr538Arg, Val539Asp, Phe540del, Phe540Ser, Asn544Ser, Ala546Thr, Ala546Asp, Phe547Ser, Pro551Gln, Leu558Pro, His572del, Gly594Val, Val613del, Val613Gly, Met619Lys, Ala620Asp, Asn622His, Asn622Lys, Asn622Lys, Gly623Arg, Gly623Asp, Val624_Val625del, Val624Met, Val625Asp, His626Arg, His626Pro, Val627SerfsX44, Thr629_Asn630insAsnValPro, Val631Asp, Arg666Ser, Arg555Trp, Arg124Ser, Asp123delins, Arg124His, Arg124Leu, Leu509Pro, Leu103_Ser104del, Val113Ile, Asp123His, Arg124Leu, and/or Thr125_Glu126del in TGFBI, for example, of Protein Accession No. Q15582; (ii) mutant KRT3 proteins comprising a mutation corresponding to Glu498Val, Arg503Pro, and/or Glu509Lys in Keratin 3 protein, for example, of Protein Accession No. P12035 or NP_476429.2; (iii) mutant KRT12 proteins with Met129Thr, Met129Val, Gln130Pro, Leu132Pro, Leu132Va, Leu132His, Asn133Lys, Arg135Gly, Arg135Ile, Arg135Thr, Arg135Ser, Ala137Pro, Leu140Arg, Val143Leu, Val143Leu, Lle391_Leu399dup, Ile 426Val, Ile426Ser, Tyr429Asp, Tyr429Cys, Arg430Pro, and/or Leu433Arg in KRT12, for example, of Protein Accession No. Q99456.1 or NP_000214.1; (iv) mutant GSN proteins with Asp214Tyr in GSN, for example, of Protein Accession No. P06396; and (v) mutant UBIAD1 proteins comprising a mutation corresponding to Ala97Thr, Gly98Ser, Asn102Ser, Asp112Asn, Asp112Gly, Asp118Gly, Arg119Gly, Leu121Val, Leu121Phe, Val122Glu, Val122Gly, Ser171Pro, Tyr174Cys, Thr175Ile, Gly177Arg, Lys181Arg, Gly186Arg, Leu188His, Asn232Ser, Asn233His, Asp236Glu, and/or Asp240Asn in UBIAD1, for example, of Protein Accession No. Q9Y5Z9. For example, a mutant sequence comprising the mutation or SNP site encodes at least a part of mutant TGFBI protein mutated by replacing Leu with Arg at amino acid position corresponding the amino acid position 509 of Protein Accession No. Q15582. In this case, a mutation at the mutation or SNP site may be responsible for encoding the mutant amino acid at amino acid position corresponding the amino acid position 509 of Protein Accession No. Q15582. As used herein, a mutation “corresponding to” a particular mutation in a human protein may include a mutation in a different species that occur at the corresponding site of the particular mutation of the human protein. Also as used herein, when a mutant protein is described to include a particular mutant, for example, of Leu509Arg, such a mutant protein may comprise any mutation that occurs at a mutant site corresponding to the particular mutant in a relevant human protein, for example, in TGFBI protein of Protein Accession No. Q15582 as described herein.

In another aspect, the present disclosure is also related to methods of altering expression of at least one gene product comprising introducing the engineered CRISPR/Cas9 system described herein into a cell containing and expressing a DNA molecule having a target sequence and encoding the gene product. The engineered CRISPR/Cas9 system can be introduced into cells using any suitable method. In some embodiments, the introducing may comprise administering the engineered CRISPR/Cas9 system described herein to cells in culture, or in a host organism.

Exemplary methods for introducing the engineered CRISPR/Cas9 system include, but are not limited to, transfection, electroporation and viral-based methods. In some cases, the one or more cell uptake reagents are transfection reagents. Transfection reagents include, for example, polymer based (e.g., DEAE dextran) transfection reagents and cationic liposome-mediated transfection reagents. Electroporation methods may also be used to facilitate uptake of the nucleic acid manipulation reagents. By applying an external field, an altered transmembrane potential in a cell is induced, and when the transmembrane potential net value (the sum of the applied and the resting potential difference) is larger than a threshold, transient permeation structures are generated in the membrane and electroporation is achieved. See, e.g., Gehl et al., Acta Physiol. Scand. 177:437-447 (2003). The engineered CRISPR/Cas9 system also may be delivered through viral transduction into the cells. Suitable viral delivery systems include, but are not limited to, adeno-associated virus (AAV), retroviral and lentivirus delivery systems. Such viral delivery systems are useful in instances where the cell is resistant to transfection. Methods that use a viral-mediated delivery system may further include a step of preparing viral vectors encoding the nucleic acid manipulation reagents and packaging of the vectors into viral particles. Other method of delivery of nucleic acid reagents include, but are not limited to, lipofection, nucleofection, microinjection, biolistics, virosomes, liposomes, immunoliposomes, polycation or lipid:nucleic acid conjugates, naked DNA, artificial virions, and agent-enhanced uptake of nucleic acids. See, also Neiwoehner et al., Nucleic Acids Res. 42:1341-1353 (2014), and U.S. Pat. Nos. 5,049,386, 4,946,787; and 4,897,355, which are herein incorporated by reference in its entirety for all purposes, and particularly for all teachings relating to reagent delivery systems. In some embodiments, the introduction is performed by non-viral vector delivery systems include DNA plasmids, RNA (e.g., a transcript of a vector described herein), naked nucleic acid, and nucleic acid complexed with a delivery vehicle, such as a liposome. Delivery can be to cells (e.g., in vitro or ex vivo administration) or target tissues (e.g., in vivo administration).

The cells that have undergone a nucleic acid alteration event (i.e., a “altered” cell) can be isolated using any suitable method. In some embodiments, the repair nucleotide molecule further comprises a nucleic acid encoding a selectable marker. In these embodiments, successful homologous recombination of the repair nucleotide molecule a host stem cell genome is also accompanied by integration of the selectable marker. Thus, in such embodiments, the positive marker is used to select for altered cells. In some embodiments, the selectable marker allows the altered cell to survive in the presence of a drug that otherwise would kill the cell. Such selectable markers include, but are not limited to, positive selectable markers that confer resistance to neomycin, puromycin or hygromycin B. In addition, a selectable marker can be a product that allows an altered cell to be identified visually among a population of cells of the same type, some of which do not contain the selectable marker. Examples of such selectable markers include, but are not limited to the green fluorescent protein (GFP), which can be visualized by its fluorescence; the luciferase gene, which, when exposed to its substrate luciferin, can be visualized by its luminescence; and β-galactosidase (β-gal), which, when contacted with its substrate, produces a characteristic color. Such selectable markers are well known in the art and the nucleic acid sequences encoding these markers are commercially available (see, e.g., Sambrook et al., Molecular Cloning: A Laboratory Manual (Cold Spring Harbor Laboratory Press 1989). Methods that employ selectable markers that can be visualized by fluorescence may further be sorted using Fluorescence Activated Cell Sorting (FACS) techniques. Isolated manipulated cells may be used to establish cell lines for transplantation. The isolated altered cells can be cultured using any suitable method to produce a stable cell line.

In another aspect, the present disclosure is related to methods of treating a disease associated with a gene mutation or single-nucleotide polymorphism (SNP) in a subject in need thereof, comprising: (a) obtaining a plurality of stem cells comprising a nucleic acid mutation in a corneal dystrophy target nucleic acid from the subject; (b) manipulating the nucleic acid mutation in one or more stem cells of the plurality of stem cells to correct the nucleic acid mutation, thereby forming one or more manipulated stem cells; (c) isolating the one or more manipulated stem cells; and (d) transplanting the one or more manipulated stem cells into the subject, wherein manipulating the nucleic acid mutation in the one or more stem cells of the plurality of stem cells includes performing any of the methods of altering expression of a gene product or of preventing, ameliorating, or treating a disease associated with a gene mutation or single-nucleotide polymorphism (SNP) in a subject as described herein.

The subject methods may include obtaining a plurality of stem cells. Any suitable stem cells can be used for the subject method, depending on the type of the disease to be treated. In certain embodiments, the stem cell is obtained from a heterologous donor. In such embodiments, the stem cells of the heterologous donor and the subject to be treated are donor-recipient histocompatible. In certain embodiments, autologous stem cells are obtained from the subject in need of the treatment for the disease. Obtained stem cells carry a mutation in a gene associated with the particular disease to be treated. Suitable stem cells include, but are not limited to, dental pulp stem cells, hair follicle stem cells, mesenchymal stem cells, umbilical cord lining stem cells, embryonic stem cells, oral mucosal epithelial stem cells and limbal epithelial stem cells.

Stem cells to be manipulated may include individual isolated stem cells or stem cells from a stem cell line established from the isolated stem cells. Any suitable genetic manipulation method may be used to correct the nucleic acid mutation in the stem cells.

In another aspect, provided herein are kits comprising the CRISPR/Cas9 system for the treatment of a disease associated with a gene mutation or single-nucleotide polymorphism (SNP). In some embodiments, the kit includes one or more sgRNAs described herein, a Cas9 nuclease and a repair nucleotide molecule that includes a wild-type allele of the mutation to be repaired as described herein. In some embodiments, the kit also includes agents that facilitate uptake of the nucleic acid manipulation by cells, for example, a transfection agent or an electroporation buffer. In some embodiments, the subject kits provided herein include one or more reagents for the detection or isolation of stem cells, for example, labeled antibodies for one or more positive stem cell markers that can be used in conjunction with FACS.

In another aspect, the present disclosure is related to an sgRNA pair, and a kit comprising the sgRNA pair comprising at least two sgRNAs for CRISPR/Cas9 system to silence a disease-causing mutation or SNP, for example, for preventing, ameliorating or treating corneal dystrophies. In some embodiments, the sgRNA pair comprises an sgRNA comprising a guide sequence for PAM-generating ancestral variation or SNP in a target gene, for example, in an intron in cis with a disease-causing mutation or SNP. In additional embodiments, the sgRNA pair comprises an sgRNA comprising a common guide sequence for PAM generating an ancestral SNP in intronic regions of a target gene.

### EXAMPLES

The following examples are presented to illustrate various embodiments of the invention. It is understood that such examples do not represent and are not intended to represent exclusive embodiments; such examples serve merely to illustrate the practice of this invention.

Mutation analysis: Mutations associated with various corneal dystrophies were analyzed to determine which were solely caused by missense mutations or in-frame indels. This analysis indicates that for the majority of K12 and TGFBI disease, nonsense or frameshifting indel mutations are not associated with disease. Furthermore, an analysis of the exome variant database confirmed that any naturally occurring nonsense, frameshifting indels or splice site mutations found in these genes are not reported to be associated with disease in these individuals.

Mutation analysis revealed that the following corneal-dystrophy genes are suitable for targeted nuclease gene therapy (Table 1).

TABLE 1
Genes and their associated corneal dystrophies that
are suitable for a CRISPR/Cas9 mediated approach.

| Gene | Associated Corneal Dystrophies |
|---|---|
| TGFBI | Avellino corneal dystrophy |
| | Reis-Bücklers corneal dystrophy |
| | Thiel-Behnke corneal dystrophy |
| | Grayson -Wilbrandt corneal dystrophy |
| | Lattice Corneal Dystrophy I & II |
| | Granular Corneal Dystrophy I, II & III |
| | Epithelial Basement Membrane Dystrophy |
| KRT3 | Meesemann Epithelial Corneal Dystrophy |
| KRT12 | Meesemann Epithelial Corneal Dystrophy |
| UBIAD1 | Schnyder corneal dystrophy |

An investigation of the suitable corneal dystrophy genes was conducted for this report to determine the number of mutations targetable by either a PAM-specific approach or a guide allele-specific approach. A PAM-specific approach requires the disease causing SNP to generate a novel PAM, whilst the allele specific approach involves the design of a guide containing the disease causing SNP. All non-disease causing SNPs in TGFBI that generate a novel PAM with a minor allele frequency (MAF) of >10% were identified and analyzed by the Benchling's online genome-editing design tool. The selection of SNPs with a MAF of >10% may provide a reasonable chance that the SNP resulting in a novel PAM will be found in cis with the disease causing mutation. Being “in cis” with the disease causing mutation refers to being on the same molecule of DNA or chromosome as the disease-causing mutation. The SNP resulting in a novel PAM may be found, for example, in intron or exon in TGFBI gene in cis with the disease-causing mutation. All variants within TGFBI were analyzed to determine whether a novel PAM was created (Table 2).

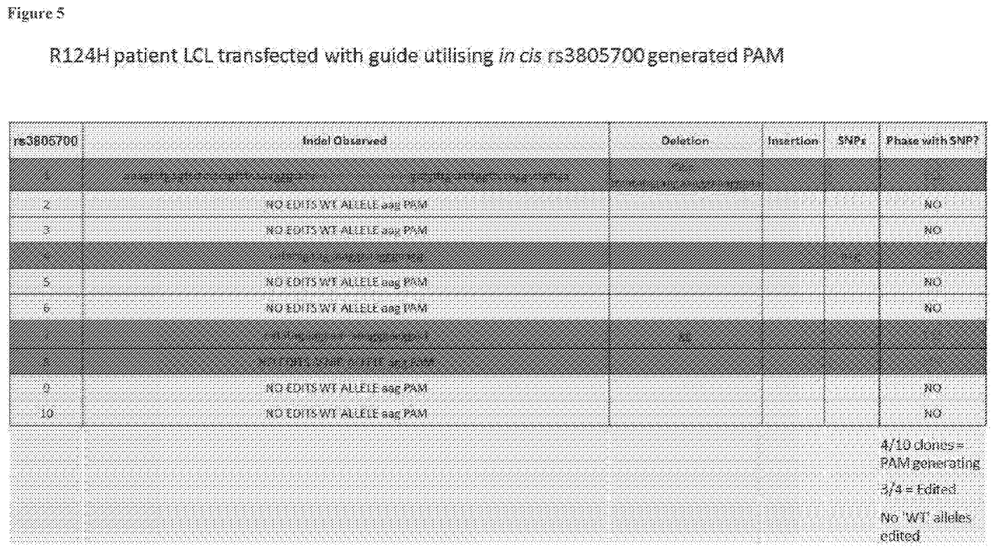

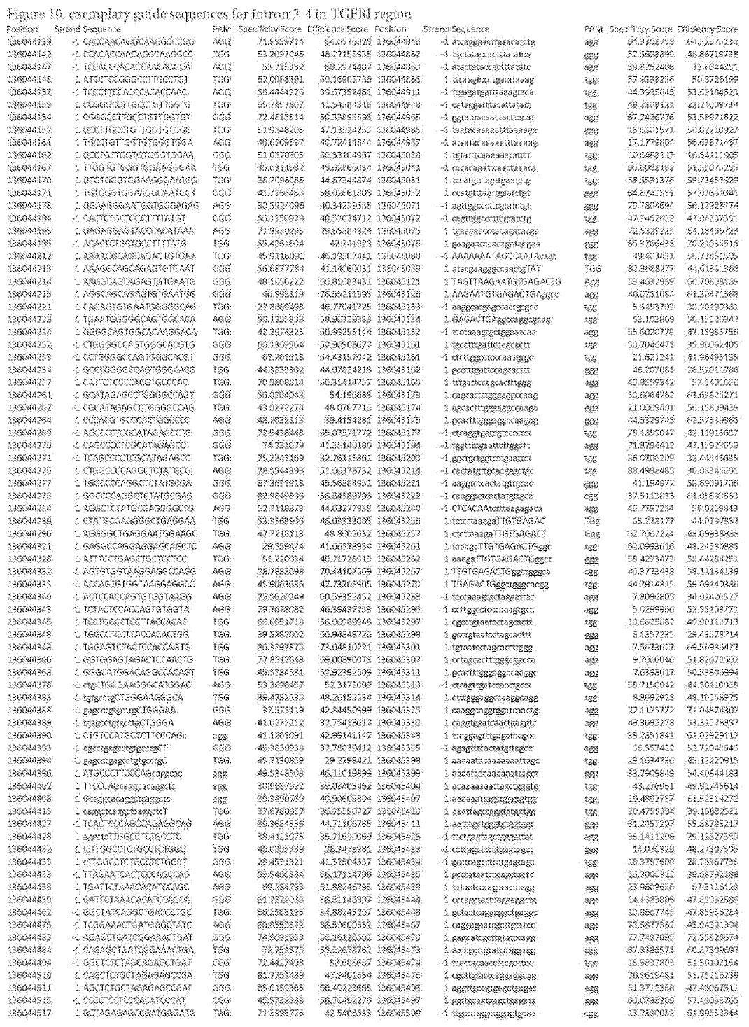

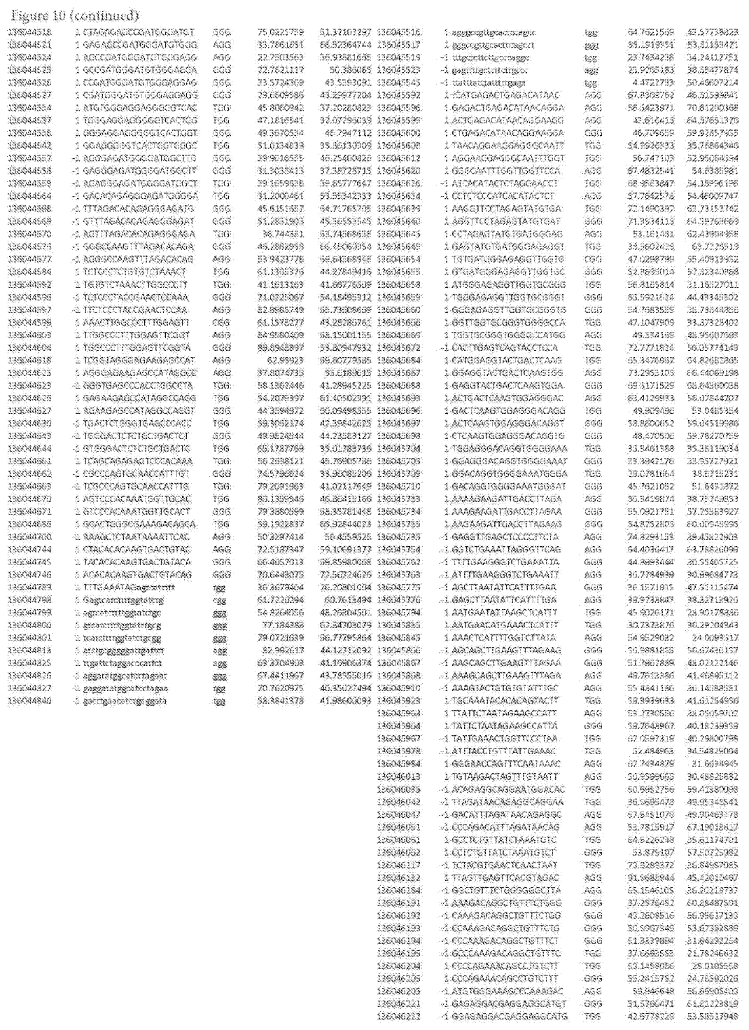

As shown in FIG. 4, the positions of the variants within TGFBI, with most of the SNPs clustered in introns. Thus, multiple TGFBI mutations located in the hotspots in exons 11, 12 and 14 may be targeted simultaneously using this approach. Therefore, a CRISPR Cas 9 system may target more than one patient or one family with a mutation. One CRISPR/Cas9 system designed in this way may be used to treat a range of TGFBI mutations. The CRISPR/Cas9 system may employ an sgRNA adjacent to a PAM site located in the flanking intron that is common to both wild-type and mutant alleles in tandem with a sgRNA adjacent to a PAM site that is specific to the mutant allele (FIG. 16). This would result in NHEJ in the intron of the wild-type allele that should have no functional effect, while in the mutant allele would result in a deletion encompassing the DNA between the two cut sites. This technique is demonstrated in leucocytes isolated from a patient with a suitable SNP profile.

TABLE 2
The variants within TGFB1 that result in a novel PAM that have a MAF of >10%.
The novel PAM is shown with the required variant indicated in red.

| | | | | | | | | Novel PAM | | | | Population Genetics | | | | | |
|---|---|---|---|---|---|---|---|---|---|---|---|---|---|---|---|---|---|
| | | | Region | Region | | | | (Required | | | | All | | | | | |
| | Num- | | Start | End | Variant | | Vari- | variant | Guide | | | Individ- | | Ameri- | East | | South |
| Region | ber | Position | Co-ordinate | Co-ordinate | Co-ordinate | SNP | ant | in red) | Sequence | Strand | MAF | uals | African | can | Asian | European | Asian |
| Exon | 1 | | 136,028,895 | 136,029,189 | | | | | | | | | | | | | |
| 1 to 2 | | Intronic | 136 209 190 | 136 033 762 | 136,032,206- | rs756462 | T/C | ccc | GAATCCA | − | 0.31 | T: 69% | 5: 56% | T: 63% | T: | T: 79% | T: |
| | | variant, | | | | | | | TGTAAGG | | | C: 31% | C: 44% | C: 37% | 64% | C: 21% | 85% |
| | | 1607 bp | | | | | | | ATCTAG | | | | | | C: | | C: |
| | | away | | | | | | | | | | | | | 36% | | 15% |
| | | from | | | | | | | | | | | | | | | |
| | | exon 2 | | | | | | | | | | | | | | | |
| Exon | 2 | | 136,033,763 | 136,033,861 | | | | | | | | | | | | | |
| Intron | 2 to | Intronic | 136,0 | 136,0 | 136,0 | rs1989972 | A/C | attcca | CAGGGCT | − | 0.43 | A: 43% | A: 26% | A: 51% | A: | A: 52% | A |
| | 3 | variant, | | | | | | | GTATTAC | | | | | | 42% | | 49% |
| | | 1945 bp | | | | | | | TGGGGC | | | | | | | | |
| | | away | | | | | | | | | | | | | | | |
| | | from | | | | | | | | | | | | | | | |
| | | exon 3 | | | | | | | | | | | | | | | |
| | | Intronic | | | | RS1989972 | A/C | cca | CAGGGCT | − | 0.43 | A: 43% | A: 26% | A: 51% | A: | A: 52% | A: |
| | | variant, | | | | | | | GTATTAC | | | | | | 42% | | 49% |
| | | 1945 bp | | | | | | | TGGGGC | | | | | | | | |
| | | away | | | | | | | | | | | | | | | |
| | | from | | | | | | | | | | | | | | | |
| | | exon 3 | | | | | | | | | | | | | | | |
| | | Intronic | | | | rs1989972 | A/C | ccc | AGGGCTG | − | 0.43 | A: 43% | A: 26% | A: 51% | A: | A: 52% | A: |
| | | variant, | | | | | | | TATTACT | | | C: 57% | C: 74% | C: 49% | 42% | C: 48% | 49% |
| | | 1945 bp | | | | | | | GGGGCT | | | | | | C: | | |
| | | away | | | | | | | | | | | | | 58% | | |
| | | from | | | | | | | | | | | | | | | |
| | | exon 3 | | | | | | | | | | | | | | | |
| | | Intronic | | | 136,043,042- | rs2805700 | A/G | agg | ATTCATA | + | 0.41 | A: 59% | A: 40% | A: 63% | A: | A: 72% | A: |
| | | variant, | | | | | | | TAGAAGA | | | G: 41% | G: 60% | G: 37% | 66% | G: 28% | 63% |
| | | 966 bp | | | | | | | AAGGAA | | | | | | G: | | G: |
| | | away | | | | | | | | | | | | | 34% | | 37% |
| | | from | | | | | | | | | | | | | | | |
| | | exon 3 | | | | | | | | | | | | | | | |
| Exon | 3 | | 136,044,058 | 136,044,122 | | | | | | | | | | | | | |
| Intron | 3 to | | 136,044,123 | 136,046,334 | | | | | | | | | | | | | |
| | 4 | | | | | | | | | | | | | | | | |
| Exon | 4 | | 136,046,335 | 136,046,495 | | | | | | | | | | | | | |
| Intron | 4 to | | 136,046,496 | 136,046,850 | | | | | | | | | | | | | |
| | 5 | | | | | | | | | | | | | | | | |
| Exon | 5 | | 136,046,851 | 136,047,015 | | | | | | | | | | | | | |
| Intron | 5 to | | 136,047,016 | 136,047,273 | | | | | | | | | | | | | |
| | 6 | | | | | | | | | | | | | | | | |
| Exon | 6 | Synony- | 136,047,274 | 136,047,420 | 136,047,250- | rs1442 | G/C | CCT | TTGCATG | − | 0.32 | C: 32% | C: 3% | C: 45% | C: | C: 47% | C: |
| | | mous | | | | | | | GTGGTCG | | | G: 68% | G: 97% | G: 55% | 37% | G: 53% | 40% |
| | | variant, | | | | | | | GCTTTC | | | | | | G: | | G: |
| | | protein | | | | | | | | | | | | | 63% | | 60% |
| | | position | | | | | | | | | | | | | | | |
| | | 217 | | | | | | | | | | | | | | | |
| Intron | 6 to | Intronic | 136,047,421 | 136,049,438 | 136,047,489- | rs764567 | A/G | cgg | TCCTGTA | + | 0.3 | A: 70% | A: 70% | A: 67% | A: | A: 74% | A: |
| | 7 | variant, | | | | | | | GGGGAAC | | | G: 30% | G: 30% | G: 33% | 75% | G: 26% | 64% |
| | | 119 bp | | | | | | | ATAGAG | | | | | | G: | | G |
| | | away | | | | | | | | | | | | | 25% | | 36% |
| | | from | | | | | | | | | | | | | | | |
| | | exon 6 | | | | | | | | | | | | | | | |
| | | Intronic | | | | rs764567 | A/G | gcgggt | CTCCTGT | + | 0.3 | A: 70% | A: 70% | A: 67% | A: | A: 74% | A: |
| | | variant, | | | | | | | AGGGGAA | | | G: 30% | G: 30% | G: 33% | 75% | G: 26% | 64% |
| | | 119 bp | | | | | | | CATAGA | | | | | | G: | | G |
| | | away | | | | | | | | | | | | | 25% | | 36% |
| | | from | | | | | | | | | | | | | | | |
| | | exon 6 | | | | | | | | | | | | | | | |
| | | Intronic | | | 136,047,6 | rs2073509 | A/G | agg | GTGTGTG | + | 0.4 | T: 60% | T: 39% | T: 63% | T: | T: 74% | T: |
| | | variant, | | | | | | | GCTGCAG | | | G: 40% | G: 61% | G: 37% | 66% | G: 26% | 63% |
| | | 268 bp | | | | | | | CAGCAC | | | | | | G: | | G: |
| | | away | | | | | | | | | | | | | 34% | | 37% |
| | | from | | | | | | | | | | | | | | | |
| | | variant | | | | | | | | | | | | | | | |
| | | Intronic | | | | rs2073509 | T/G | ggg | TGTGTGG | + | 0.4 | T: 60% | T: 39% | T: 63% | T: | T: 74% | T: |
| | | variant, | | | | | | | CTGCAGC | | | G: 40% | G: 61% | G: 37% | 66% | G: 26% | 63% |
| | | 268 bp | | | | | | | AGCACA | | | | | | G: | | G: |
| | | away | | | | | | | | | | | | | 34% | | 37% |
| | | from | | | | | | | | | | | | | | | |
| | | variant | | | | | | | | | | | | | | | |
| | | Intronic | | | | rs2073509 | T/G | cagggt | GGTGTGT | + | 0.4 | T: 60% | T: 39% | T: 63% | T: | T: 74% | T: |
| | | variant, | | | | | | | GGCTGCA | | | G: 40% | G: 61% | G: 37% | 66% | G: 26% | 63% |
| | | 268 bp | | | | | | | GCAGCA | | | | | | G: | | G: |
| | | away | | | | | | | | | | | | | 34% | | 37% |
| | | from | | | | | | | | | | | | | | | |
| | | variant | | | | | | | | | | | | | | | |
| | | Intronic | | | 136,048,154- | rs2073511 | T/C | cct | GGAGAGG | − | 0.4 | T: 60% | T: 39% | T: 63% | T: | T: 74% | T: |
| | | variant, | | | | | | | AGCTTAG | | | C: 40% | C: 61% | C: 37% | 66% | C: 26% | 63% |
| | | 784 bp | | | | | | | ACAGCG | | | | | | C: | | C: |
| | | away | | | | | | | | | | | | | 34% | | 37% |
| | | from | | | | | | | | | | | | | | | |
| | | variant | | | | | | | | | | | | | | | |
| | | Intronic | | | 136,048,704- | rs916951 | A/G | cgg | GTAATAG | + | 0.37 | A: 63% | A: 95% | A: 53% | A: | A: 49% | A: |
| | | variant, | | | | | | | CAAAGGC | | (G) | G: 37% | G: 5% | G: 47% | 58% | G: 51% | 50% |
| | | 685 bp | | | | | | | TCAGGG | | | | | | G: | | G: |
| | | from | | | | | | | | | | | | | 42% | | 50% |
| | | exon 7 | | | | | | | | | | | | | | | |
| Exon | 7 | | 136,049,439 | 136,049,580 | | | | | | | | | | | | | |
| Intron | 7 to | Intronic | 136,049,581 | 136,052,906 | 136,050,039- | rs1137550 | T/C | actctg | ATCCGCC | − | 0.37 | T: 63% | T: 52% | T: 64% | T: | T: 74% | T: |
| | 8 | variant, | | | | | | | CACCTTG | | | C: 37% | C: 48% | C: 36% | 66% | C: 26% | 63% |
| | | 590 bp | | | | | | | TCCTCC | | | | | | C: | | C: |
| | | from | | | | | | | | | | | | | 34% | | 37% |
| | | exon 7 | | | | | | | | | | | | | | | |
| Exon | 8 | Synony- | 136,052,907 | 136,053,119 | 136,052,924 | rs1054124 | A/G | TGG | CATCGTT | + | 0.39 | A: 61% | A: 46% | A: 64% | A: | A: 74% | A: |
| | | mous | | | | | | | GCGGGGC | | | G: 39% | G: 54% | G: 36% | 66% | G: 26% | 63% |
| | | variant, | | | | | | | TGTCTG | | | | | | G: | | G: |
| | | protein | | | | | | | | | | | | | 34% | | 37% |
| | | position | | | | | | | | | | | | | | | |
| | | 327 | | | | | | | | | | | | | | | |
| Intron | 8 to | Intronic | 136,053,120 | 136,053,942 | 136,053,686- | rs6889640 | C/A | actctc | CCAGCTC | − | 0.37 | A: 37% | A: 54% | A: 24% | A: | A: 26% | A: |
| | 9 | variant, | | | | | | | AGGAGGA | | | C: 63% | C: 46% | C: 76% | 34% | C: 74% | 37% |
| | | 207 bp | | | | | | | GAGGGAG | | | | | | C: | | C: |
| | | from | | | | | | | | | | | | | 66% | | 63% |
| | | exon 9 | | | | | | | | | | | | | | | |
| Exon | 9 | | 136,053,943 | 136,054,080 | | | | | | | | | | | | | |
| Intron | 9 to | | 136,054,081 | 136,054,715 | | | | | | | | | | | | | |
| | 10 | | | | | | | | | | | | | | | | |
| Exon | 10 | | 136,054,716 | 136,054,861 | | | | | | | | | | | | | |
| Intron | 10 | Intronic | 136,054,862 | 136,055,679 | 136,055,587- | rs6860369 | A/G | ggg | CAAATCA | + | 0.4 | A: 60% | A: 33% | A: 75% | A: | A: 74% | A: |
| | to | variant, | | | | | | | GGAGGCC | | | G: 40% | G: 67% | G: 25% | 66% | G: 26% | 63% |
| | 11 | 43 bp | | | | | | | CCTCGT | | | | | | G: | | G: |
| | | from | | | | | | | | | | | | | 34% | | 37% |
| | | exon 11 | | | | | | | | | | | | | | | |
| Exon | 11 | | 136,055,680 | 136,055,816 | | | | | | | | | | | | | |
| Intron | 11 | | 136,055,817 | 136,056,664 | | | | | | | | | | | | | |
| | to | | | | | | | | | | | | | | | | |
| | 12 | | | | | | | | | | | | | | | | |
| Exon | 12 | | 136,056,665 | 136,056,795 | | | | | | | | | | | | | |
| Intron | 12 | Intronic | 136,056,796 | 136,059,089 | 136,057,458- | rs6871571 | A/G | ttgaat | TGCAGCC | + | 0.42 | A: 58% | A: 33% | A: 63% | A: | A: 74% | A: |
| | to | variant, | | | | | | | TGTGTTG | | | G: 42% | G: 67% | G: 37% | 66% | G: 26% | 63% |
| | 13 | 713 bp | | | | | | | GGAGGA | | | | | | G: | | G: |
| | | from | | | | | | | | | | | | | 34% | | 37% |
| | | exon 12 | | | | | | | | | | | | | | | |
| Exon | 13 | | 136,059,090 | 136,059,214 | | | | | | | | | | | | | |
| Intron | 13 | Intronic | 136,059,215 | 136,060,833 | 136,059,694- | rs6893691 | A/G | cgg | AATCTCC | + | 0.39 | A: 39% | A:11 % | A: 49% | A: | A: 52% | A: |
| | to | variant, | | | | | | | CTGGCTG | | | G: 61% | G: 89% | G: 51% | 43% | G: 48% | 51% |
| | 14 | 530 bp | | | | | | | CACCTG | | | | | | G: | | G: |
| | | from | | | | | | | | | | | | | 57% | | 49% |
| | | exon 13 | | | | | | | | | | | | | | | |
| | | Intronic | | | 136,059,804- | rs1990199 | G/C | cca | TGCATAT | − | 0.39 | C: 61% | C: 89% | C: 51% | C: | C: 48% | C: |
| | | variant, | | | | | | | CTTCCTA | | | G: 39% | G: 11% | G: 49% | 57% | G: 52% | 49% |
| | | 640 bp | | | | | | | TGCTCC | | | | | | G: | | G: |
| | | from | | | | | | | | | | | | | 43% | | 51% |
| | | exon 13 | | | | | | | | | | | | | | | |
| | | Intronic | | | 136,060,125- | rs6894815 | G/C | ccc | GAGACTG | − | 0.42 | C: 58% | C: 76% | C: 50% | C: | C: 48% | C: |
| | | variant, | | | | | | | AGACTGA | | | G: 42% | G: 24% | G: 50% | 57% | G: 52% | 49% |
| | | 659 bp | | | | | | | AGACAG | | | | | | G: | | G: |
| | | from | | | | | | | | | | | | | 43% | | 51% |
| | | exon 14 | | | | | | | | | | | | | | | |
| | | Intronic | | | 136,060,553- | rs1006447 | T/G | cgg | TGCCTGT | + | 0.42 | T: 42% | T: 23% | T: 50% | T: | T: 52% | T: |
| | | variant, | | | | 8 | | | AATCACA | | | G: 58% | G: 77% | G: 50% | 43% | G: 48% | 51% |
| | | 230 bp | | | | | | | GCTACT | | | | | | G: | | G: |
| | | from | | | | | | | | | | | | | 57% | | 49% |
| | | exon 14 | | | | | | | | | | | | | | | |
| Exon | 14 | | 136,060,834 | 136,060,936 | | | | | | | | | | | | | |
| Intron | 14 | Intronic | 136,060,937 | 136,061,499 | 136,060,903- | rs6880837 | T/C | cca | TCTCTCC | − | 0.41 | T: 41% | T: 24% | T: 49% | T: | T: 50% | T: |
| | to | variant, | | | | | | | ACCAACT | | | C: 59% | C: 76% | C: 51% | 41% | C: 50% | 48% |
| | 15 | 44 bp | | | | | | | GCCACA | | | | | | C: | | C: |
| | | from | | | | | | | | | | | | | 59% | | 52% |
| | | exon 14 | | | | | | | | | | | | | | | |
| Exon | 15 | | 136,061,500 | 136,061,579 | | | | | | | | | | | | | |
| Intron | 15 | | 136,061,580 | 136,062,662 | | | | | | | | | | | | | |
| | to | | | | | | | | | | | | | | | | |
| | 16 | | | | | | | | | | | | | | | | |
| Exon | 16 | | 136,062,663 | 136,062,687 | | | | | | | | | | | | | |
| Intron | 16 | | 136,062,688 | 136,063,185 | | | | | | | | | | | | | |
| | to | | | | | | | | | | | | | | | | |
| | 17 | | | | | | | | | | | | | | | | |
| Exon | 17 | | 136,063,817 | 136,063,185 | | | | | | | | | | | | | |

### Confirming Allele-Specific Indels

EBV transformation of lymphocytes: A sample of 5 ml of whole blood was taken and place in a sterile 50 ml Falcon tube. An equal volume of RPMI media containing 20% foetal calf serum was added to the whole blood—mix by gently inverting the tube. 6.25 ml of Ficoll-Paque PLUS (GE Healthcare cat no. 17-1440-02) was placed in a separate sterile 50 ml Falcon tube. 10 ml of blood/media mix was added to the Ficoll-Paque. The tube was spun at 2000 rpm for 20 min at room temperature. The red blood cells formed at the bottom of the tube above which was the Ficoll layer. The lymphocytes formed a layer on top of the Ficoll layer, while the top layer was the medium. A clean sterile Pastette was inserted to draw off the lymphocytes, which were placed in a sterile 15 ml Falcon tube. The lymphocytes were centrifuged and washed. EBV aliquot was thawed and added to resuspended lymphocytes, and the mixture was incubated for 1 hour at 37 degrees C. (infection period). RPMI, 20% FCS media and 1 mg/ml phytohaemagglutinin were added to EBV treated lymphocytes, and the lymphocytes were placed on a 24-well plate.

Electroporation of EBV Transformed Lymphocytes (LCLs): CRISPR constructs (with either GFP or mCherry co-expressed) were added to suspended EBV transformed lymphocytes cells, and the mixture was transferred to an electroporation cuvette. Electroporation was performed, and 500 μl pre-warmed RPMI 1640 media containing 10% FBS was added to the cuvette. The contents of the cuvette was transferred to a 12 well plate containing the remainder of the pre-warmed media, and 6 hours post nucleofection, 1 ml of media was removed and was replaced with fresh media.

Cell sorting of GFP+ and/or mCherry+ Live cells: 24 hours post nucleofection, 1 ml of media was removed and the remaining media containing cells was collected in a 1.5 ml Eppendorf. The cells were centrifuged and resuspended in 200 ul PBS add 50 ul eFlouro 780 viability stain at 1:1000 dilution. After another centrifuge, the cells were resuspended in filter sterile FACS buffer containing 1×HBSS (Ca/Mg++ free), 5 mM EDTA, 25 mM HEPES pH 7.0, 5% FCS/FBS (Heat-Inactivated) and 10 units/mL DNase II. Cells were sorted to isolate live GFP+ and/or mCherry+ cells and were collected in RPMI+20% FBS. Cells were expanded, and DNA was extracted from the cells.

Isolation of single alleles for sequencing: QIAmp DNA Mini Kit (Qiagen) was used to isolate DNA, PCR was used across the region targeted by CRISPR/Cas9. Specific amplification was confirmed by gel electrophoresis, and the PCR product was purified. The PCR product was blunt ended and ligated into pJET1.2/blunt plasmid from the Clonejet Kit (Thermo Scientific). The ligation mixture was transformed into competent DH5α cells. Single colonies were picked, and Sanger Sequencing was performed to confirm edits. The resulting data is shown in FIG. 5.

### Dual-Cut Approach

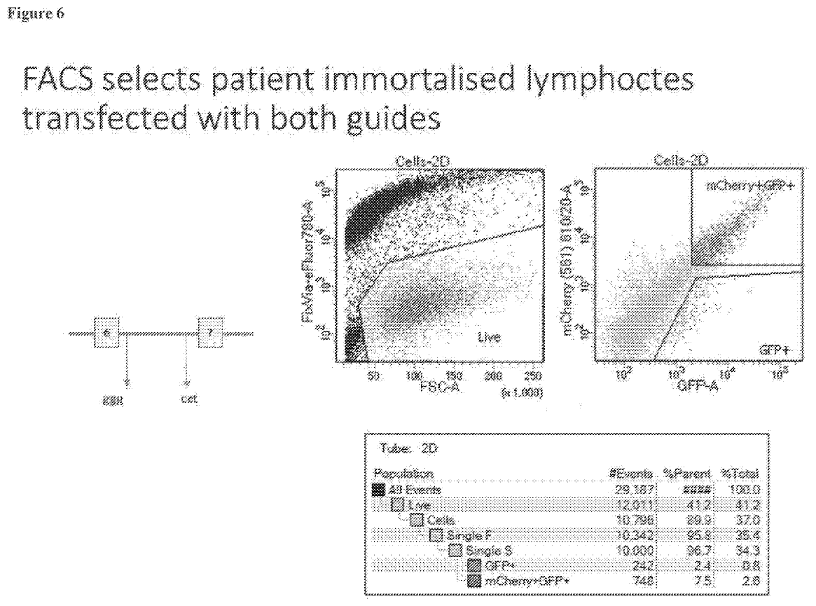

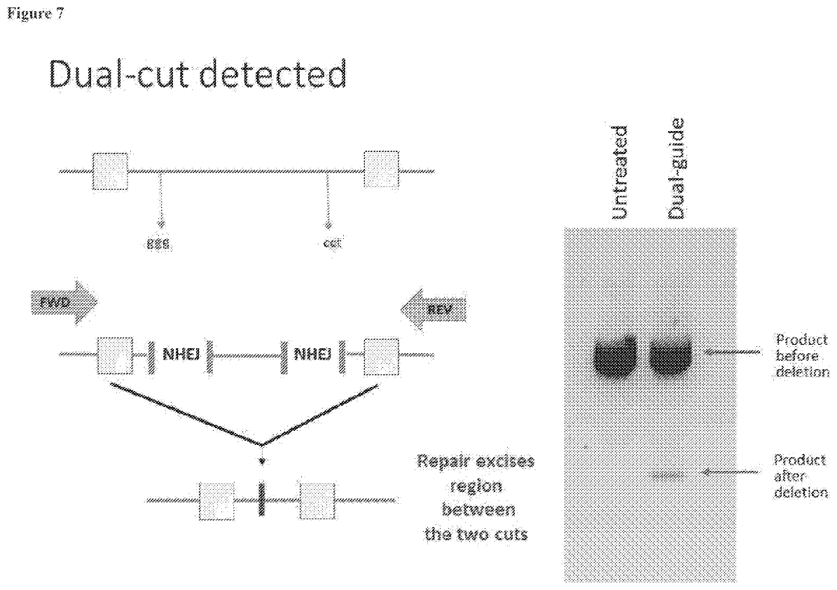

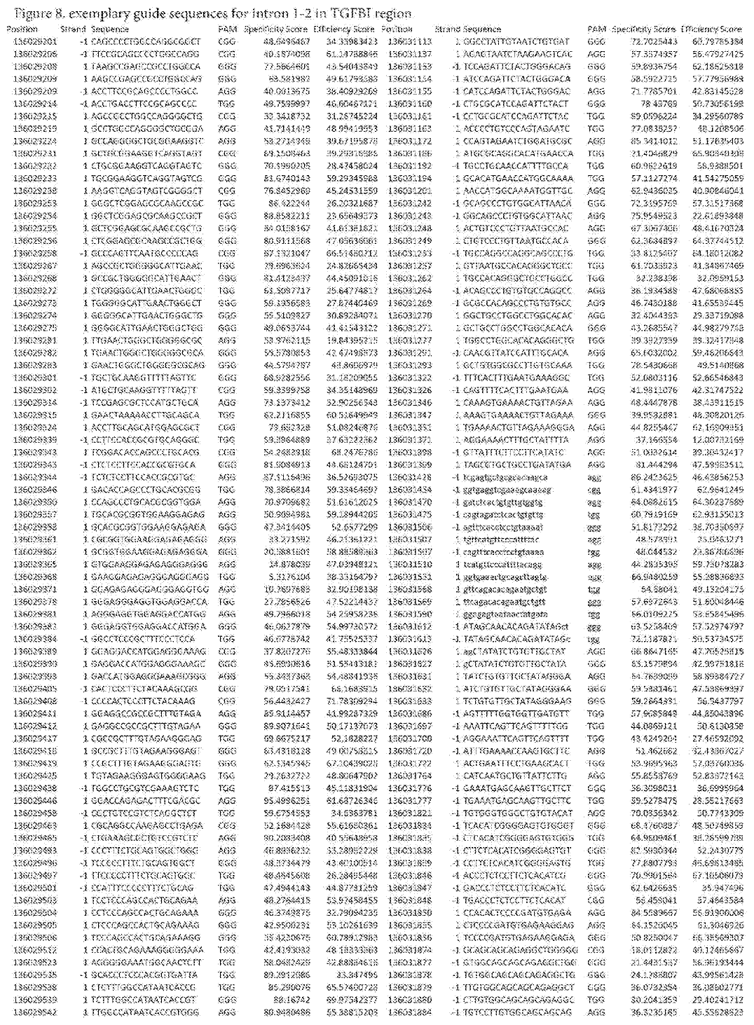

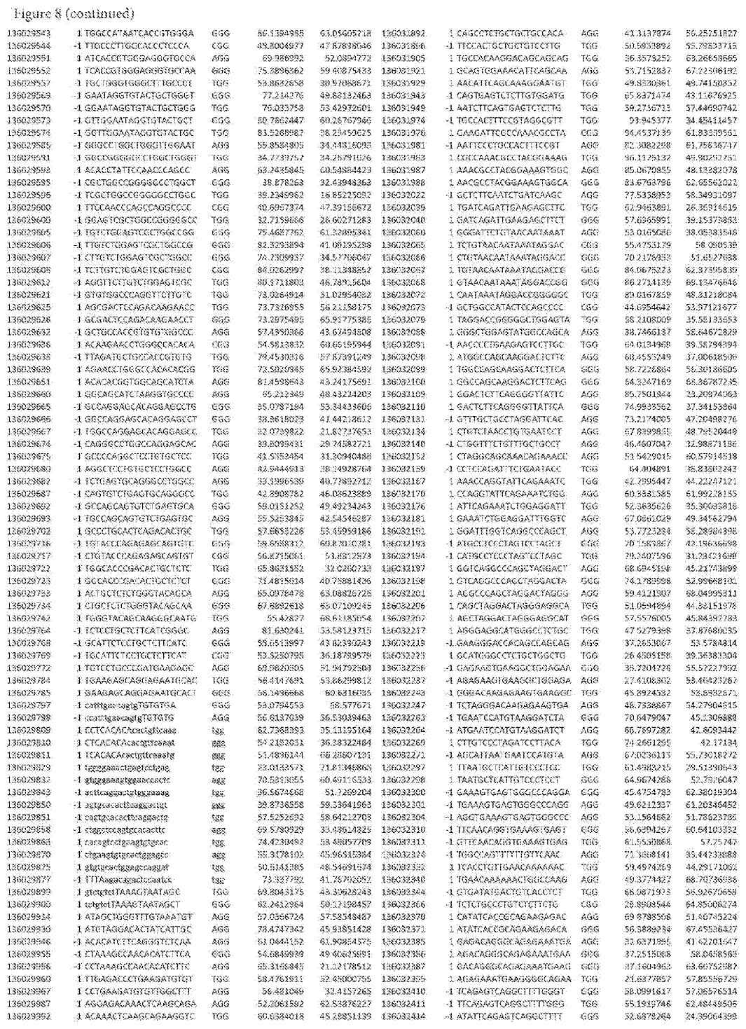

Two CRISPR plasmids were transfected into the lymphocyte cell lines (LCLs), one tagged with mCherry the other tagged with GFP. Positive cells were sorted for both mCherry and GFP, collecting 2.6% of the total population. The cells were then allowed to repair and expand, and the genomic DNA was isolated (FIG. 6). The dual-cut was indeed detected as shown in FIG. 7. Additional exemplary common guides in intronic regions of TGFBI gene, which may be used to treat corneal dystrophies, are listed in FIGS. 8-23.

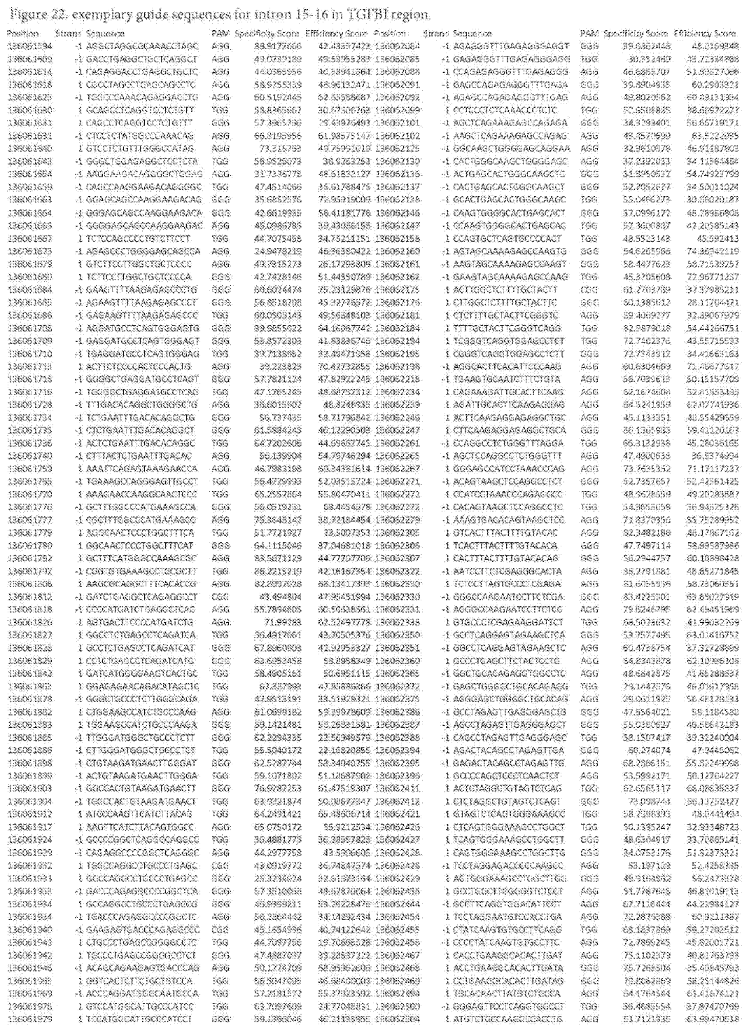

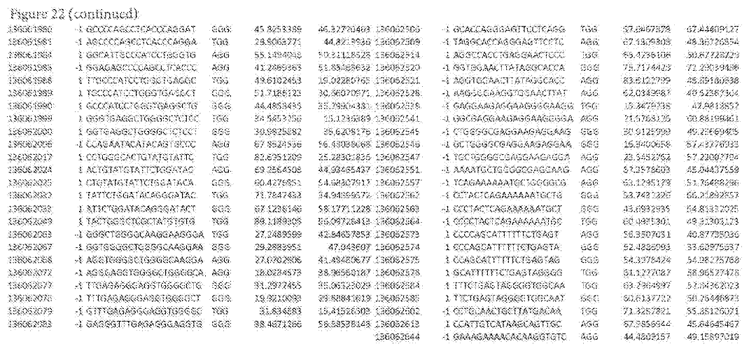

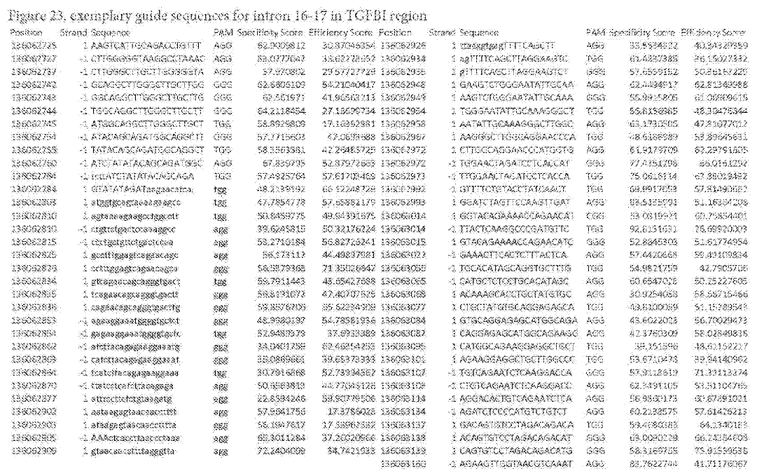

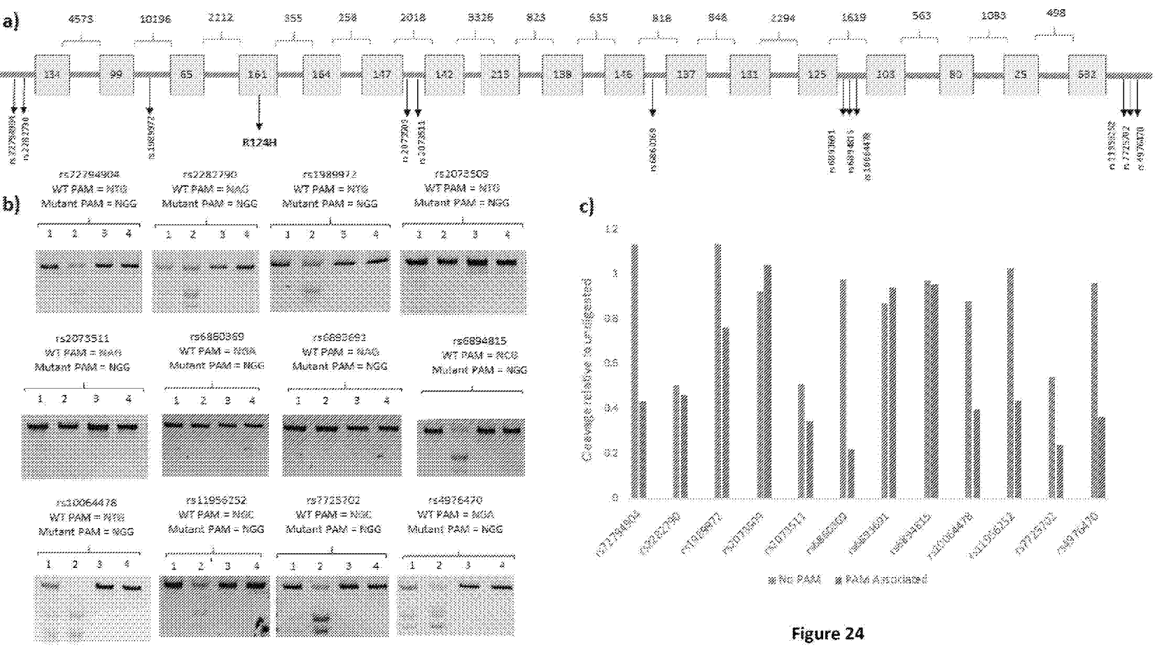

TGFBI gene editing: SNPs across the TGFBI locus suitable for ASNIP gene editing were identified as shown in FIG. 24 (section a). We identified a small set of targets within the TGFBI locus that are common to many TGFBI patients. All ASNIP SNPs were in intronic regions that do not code, so a single cut here was predicted to have no functional effect as the sequence required to produce the TGFBI protein will still be intact.

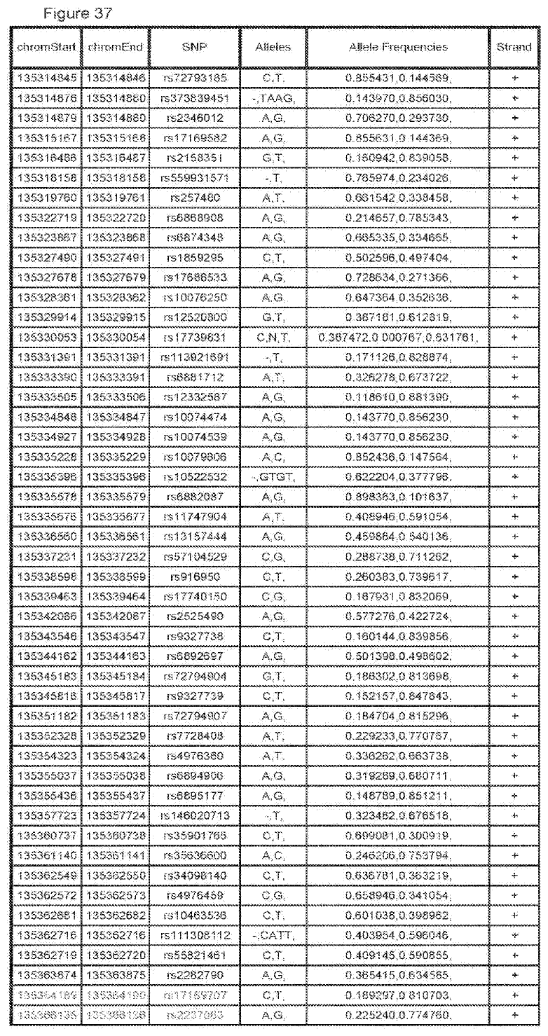

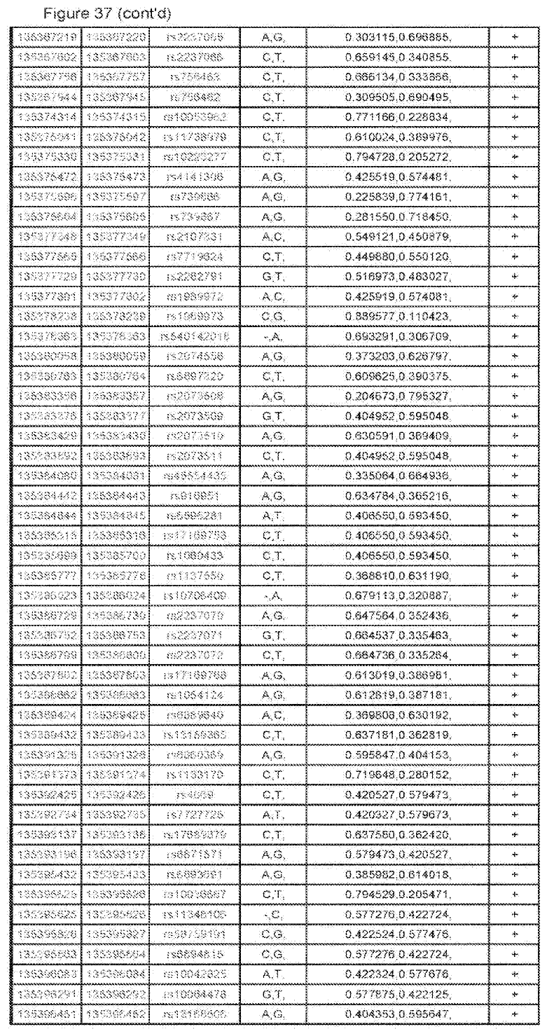

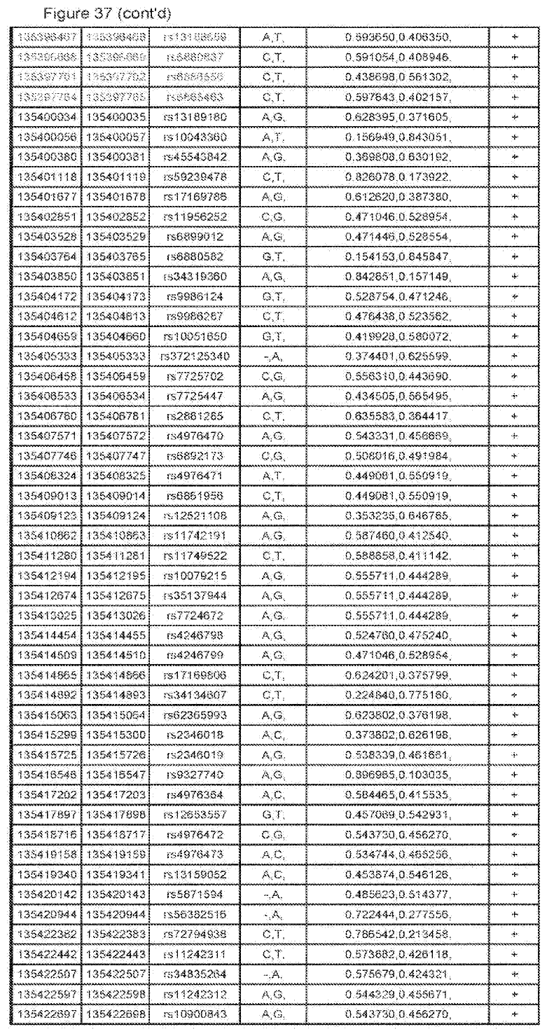

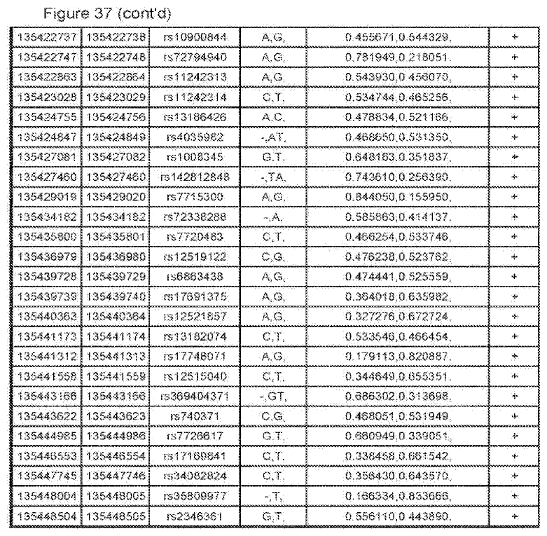

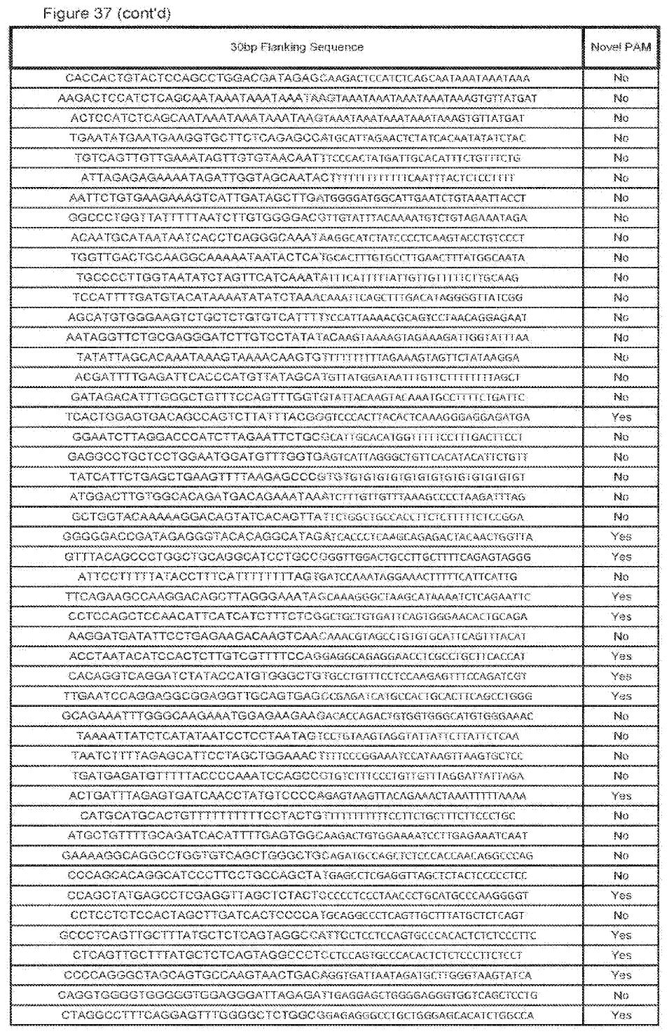

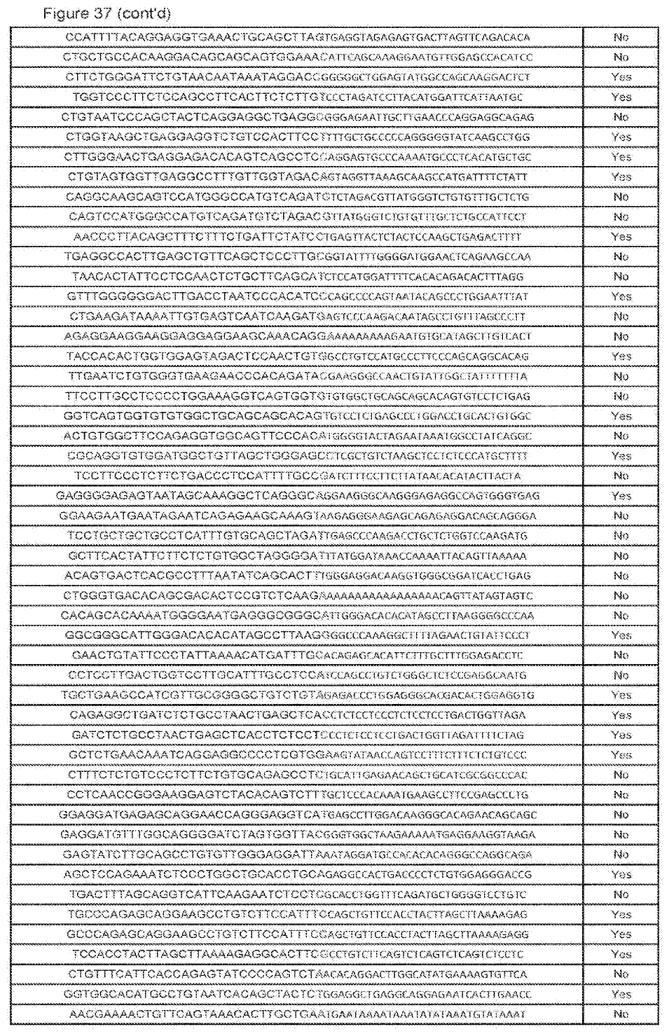

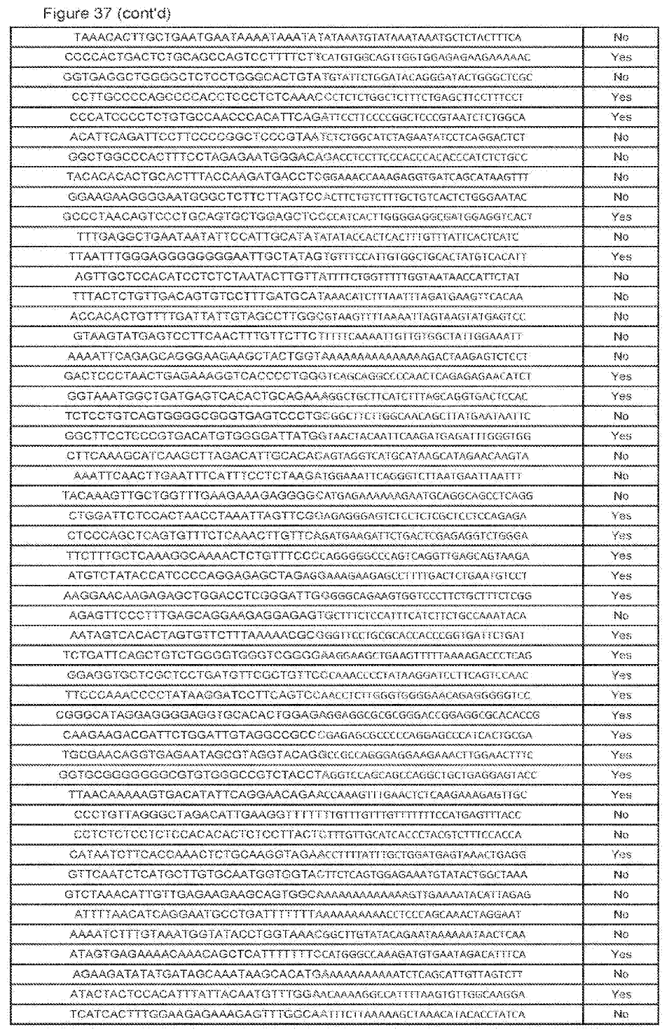

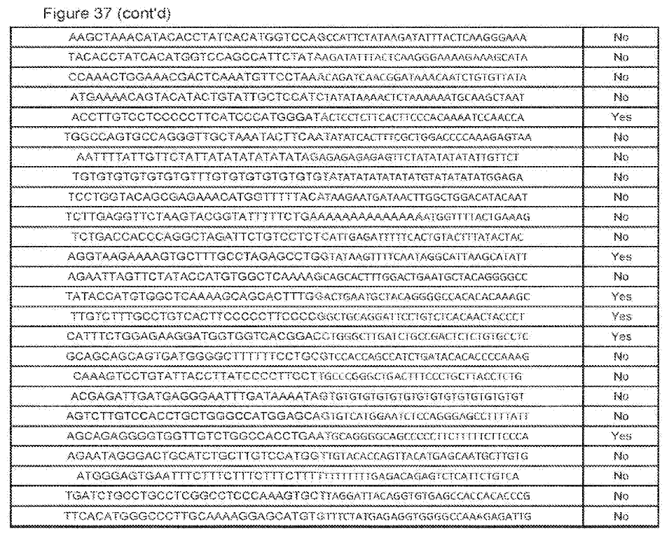

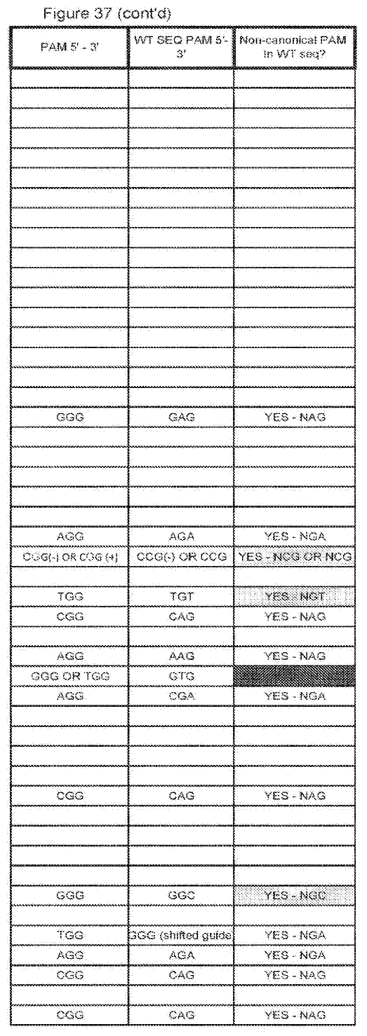

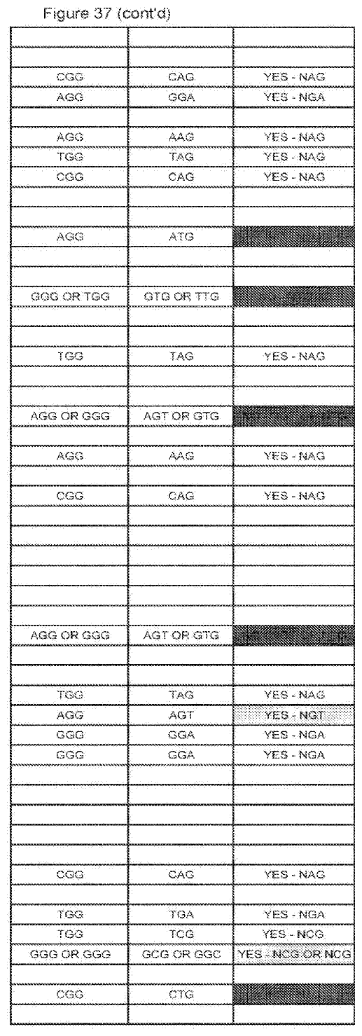

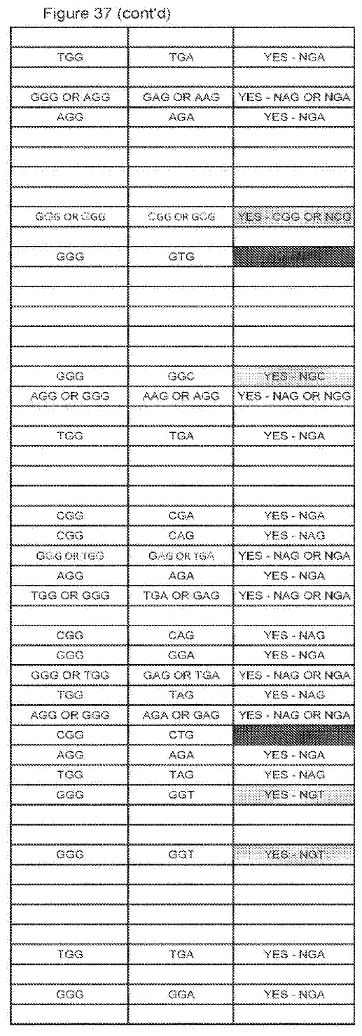

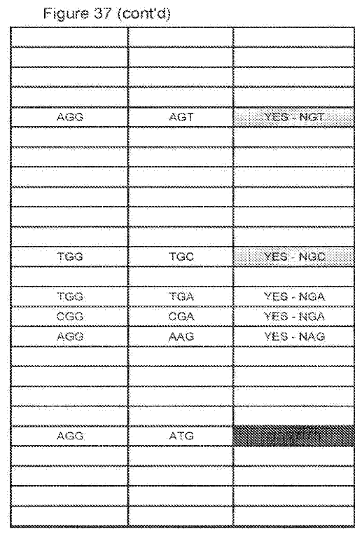

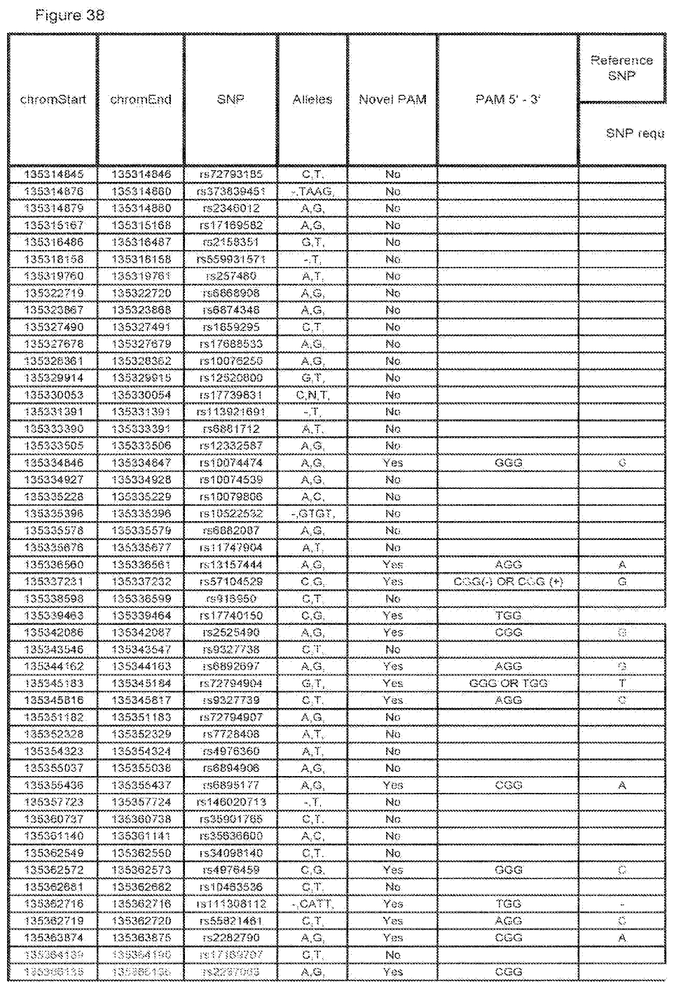

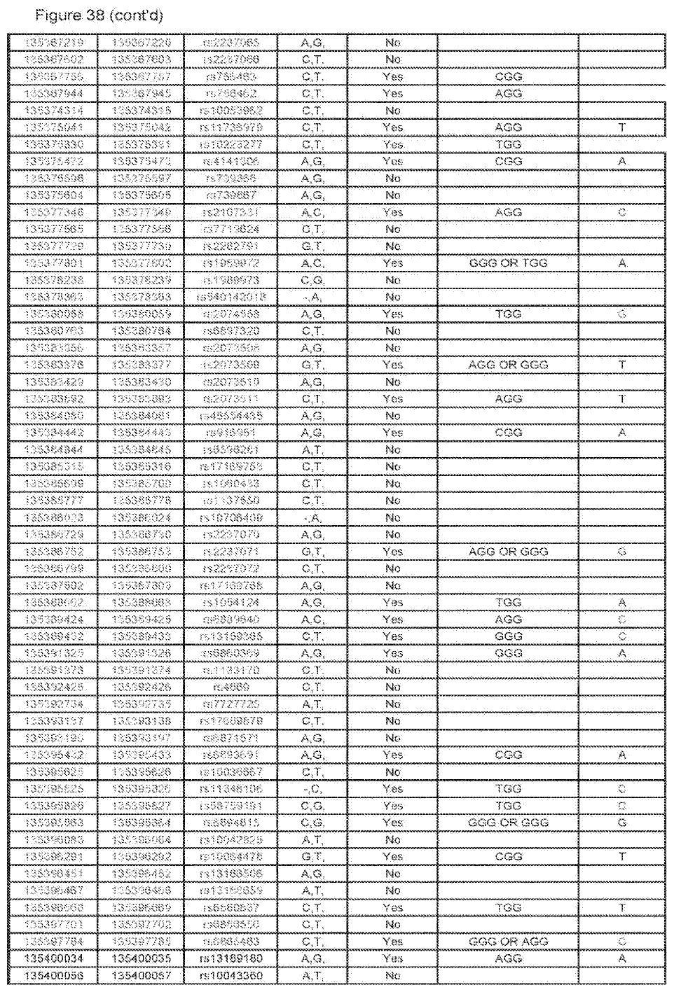

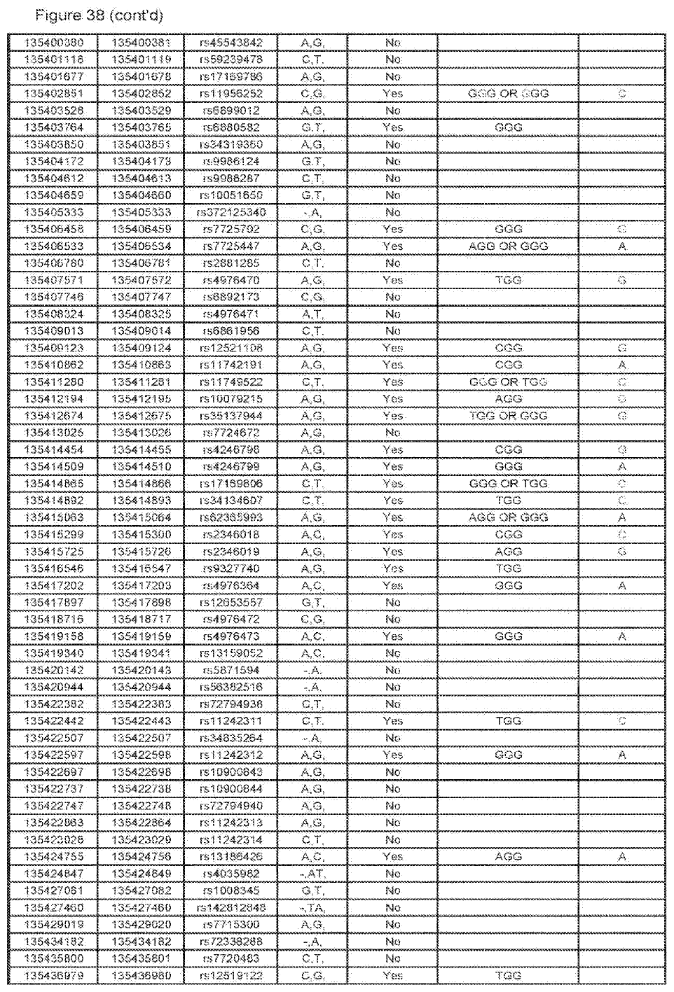

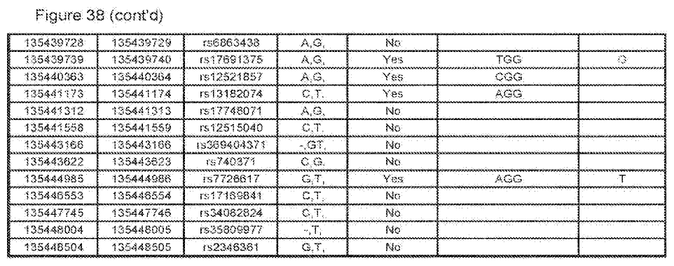

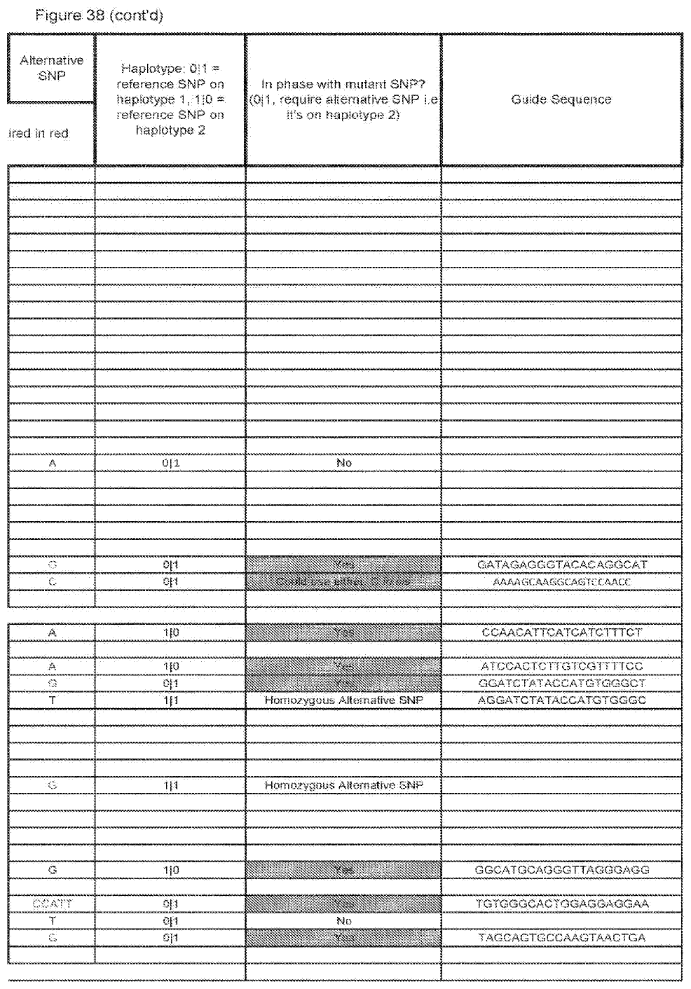

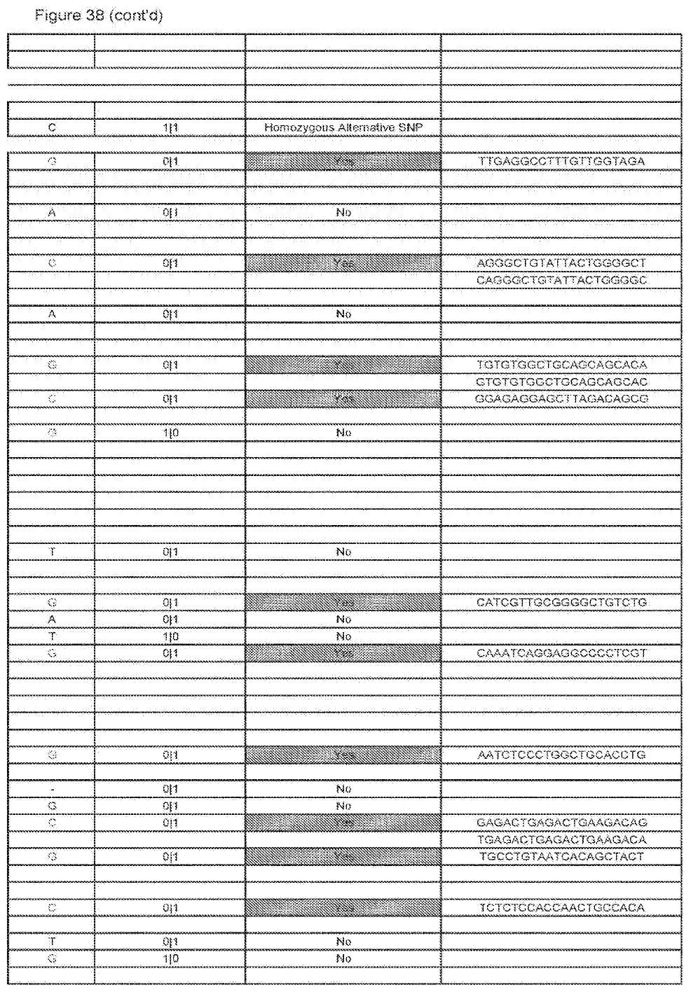

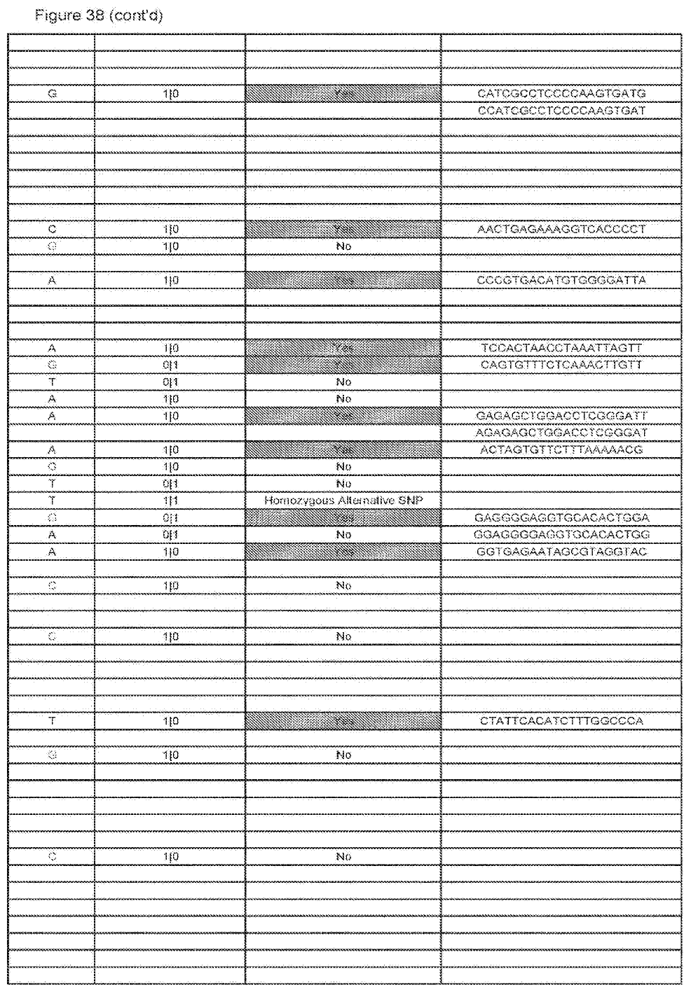

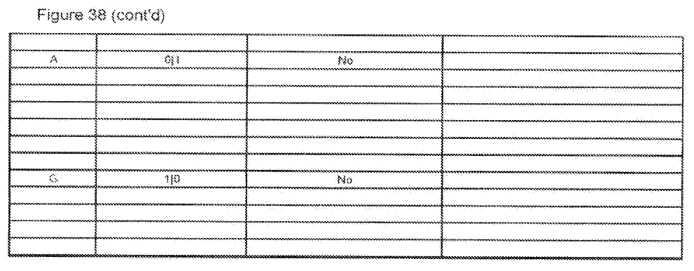

Out of SNPs containing a PAM on only one allele (FIG. 37), SNPs associated with a PAM on only one allele that lie in cis with the patient's R124H mutations were identified (FIG. 38). Various SNPs were tested as targets for Cas9/sgRNA in an in vitro cleavage assay demonstrated both on-target activity and specificity for one version of the SNP (allele) as shown in FIG. 24 (section b). Each SNP (rs72794904, rs2282790, rs1989972, rs6894815) generated a novel PAM on the same allele in which the disease-causing mutation is present. For each SNP, an in vitro digestion was performed. DNA templates were generated containing the sequence of either allele (one has a novel PAM present the other has no PAM), and this sequence was digested with Cas9 protein complexed with the targeting sgRNA. Guide sequences for the tested targets are shown in Table 3.

TABLE 3
Guide Sequences for Selected Target Mutations

| SNP | Guide Sequences (5′-3′) |
|---|---|
| rs72794904 | GGATCTATACCATGTGGGCT |
| rs2282790 | TAGCAGTGCCAAGTAACTGA |
| rs1989972 | AGGGCTGTATTACTGGGGCT |
| rs2073509 | GTGTGTGGCTGCAGCAGCAC |
| rs2073511 | GGAGAGGAGCTTAGACAGCG |
| rs6860369 | CAAATCAGGAGGCCCCTCGT |
| rs6893691 | AATCTCCCTGGCTGCACCTG |
| rs6894815 | GAGACTGAGACTGAAGACAG |
| rs10064478 | TGCCTGTAATCACAGCTACT |
| rs11956252 | CATCGCCTCCCCAAGTGATG |
| rs7725702 | AACTGAGAAAGGTCACCCCT |
| rs4976470 | CCCGTGACATGTGGGGATTA |

After incubation, digestion products were run on an agarose gel to see the cutting activity of each guide and specificity between the two alleles and intensity of the digested products revealed the in vitro specificity of each guide. Of the 12 ASNIP guides tested, 8 appeared to preferentially cleave the PAM associated allele while 4 appeared to have little activity at either the ‘PAM associated’ or ‘No PAM’ allele. SNPs generating a non-canonical PAM, which is a PAM sequence other than NGG that can still act as a weak PAM for S. pyogenes Cas9 such as NAG or NGA on the ‘No PAM present’ allele, only conferred partial discrimination at best.

5′-NAG-3′ can act as a non-canonical PAM (i.e. it is NOT equal to the wild-type 5′-NGG-3′ PAM but when present it can act as a PAM with a lower frequency). Although the tested SNP generated a PAM, if a non-canonical PAM is on the ‘no PAM allele,’ then cleave can happen on the ‘no PAM allele,’ even though the ‘no PAM allele’ does not include the 5′-NGG-3′ PAM. Indeed, in the in vitro digests, when a non-canonical PAM (NAG/NGA) was present, discrimination was poor. But when NGT/NTG/NGC/NCG was present on the ‘no PAM allele,’ better discrimination was obtained with Cas9.

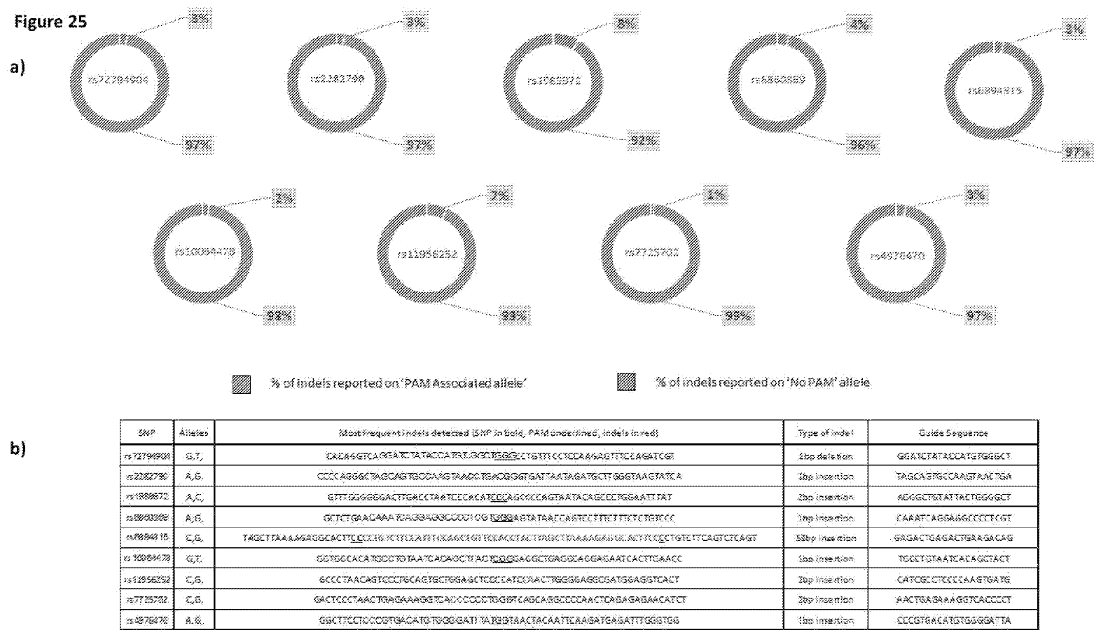

On-target activity and specificity was confirmed in a cell line derived from a GCD2 patient with a R124H TGFBI mutation as shown in FIG. 24 (section c). Specifically, each of the same guides (rs72794904, rs2282790, rs1989972, rs6860369, rs6894815, rs10064478, rs11956252, rs7725702, rs4976470) was complexed with Cas9 and this complex was transfected into a lymphocyte cell line generated from a R124H GCD2 corneal dystrophy patient. Genomic DNA was extracted from these cells 48 hours later. The region targeted by Cas9 was amplified and sent for next generation sequencing (NGS). Deletion of the intervening sequence was demonstrated between pairs of the SNPs when cells were treated with pairs of SNP-targeting sgRNAs. With each guide, more than 92% of the indels occurred on the allele with the novel PAM (FIG. 25, section a) On average, in the 9 guides tested 96.3% indels occurred on the allele with the novel PAM while only 3.7% of the indels occurred on the ‘no PAM allele.’ The results showed that all 9 guides were highly specific for the mutant allele, including even those containing a non-canonical PAM on the ‘no PAM allele.’

In addition, ASNIP guide rs6860369 was inactive in the in vitro screen but was active in a cell line. For 8 out 9 ASNIP guides tested, the predominant indels observed were 1 or 2 bp insertions, which occurred 3 or 4 bp upstream of the PAM (FIG. 25, section b).

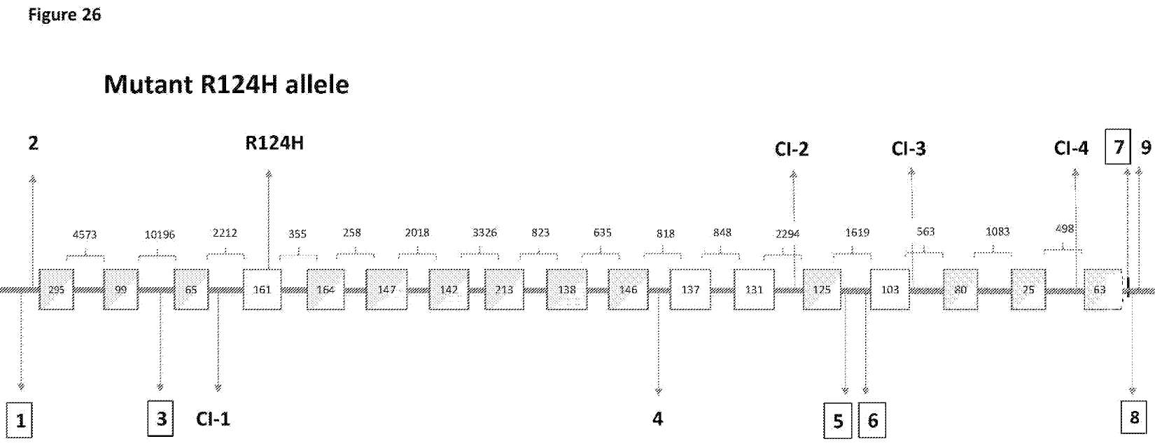

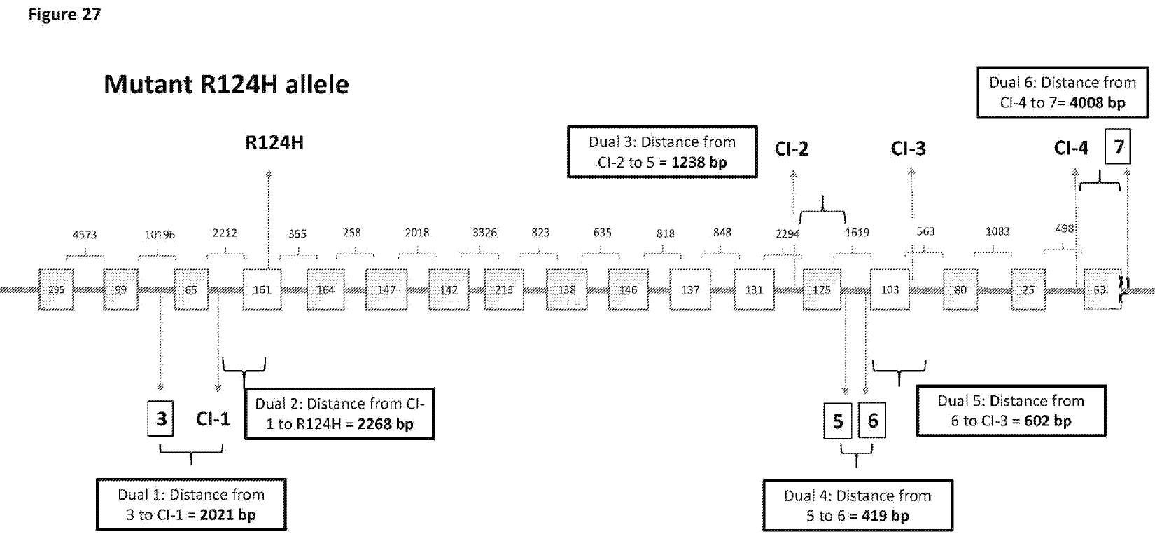

FIG. 26 indicates the sizes of the exons (coding) and introns (non-coding) in base pairs. Most of the original ASNIP guides were a substantial distance apart. FIG. 26 illustrates locations of additional common-intronic guides, CI-1 through CI-4, that were tested. The additional common-intronic guides, CI-1 through CI-4, will cut both alleles, but will be used alongside an allele-specific ASNIP guide. Thus, a dual-cut that will have a functional effect will only occur when both cuts happen in the same cell. FIG. 27 depicts six different dual combinations tested.

DNA extracted from the R124H cells transfected with various dual combinations was subjected to qPCR, normalized to a non-targeted gene elsewhere in the genome and to the untreated cell line. Any reduction in PCR product in the treated cells would be indicative of productive deletion.

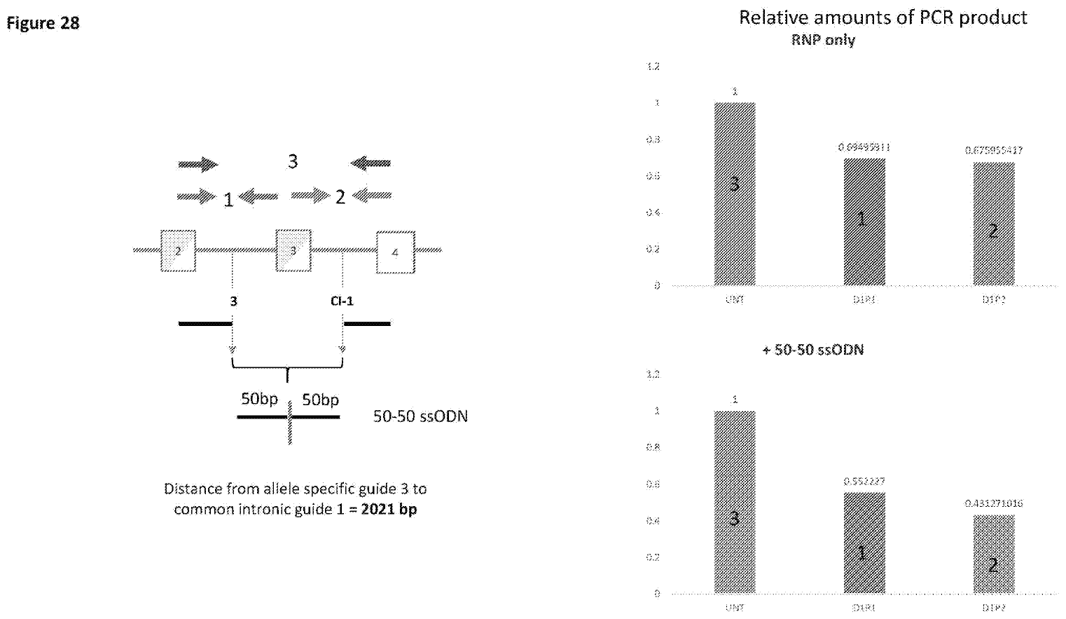

The left illustration in FIG. 28, for example, is a diagram showing where the guides are in relation to each other and how the assay to detect the presence of the dual cut works. For dual combination 1, shown in FIG. 28, the two PAM sites were 202 bp apart. The arrows indicate a PCR reaction and correspond to on the graph on the right. For PCR “1” shown by red it amplifies target region 1 and PCR “2” amplifies target region 2 and PCR “3” amplifies the expected dual cut, therefore due to amplification distance we should only get a product if the dual cut occurred. The top graph on the right shows cells transfected with 2 SNPs for each guide. PCR 1 was reduced by 39% compared to UNT (PCR3) and PCR 2 was reduced by 33%. To increase the dual cut frequency, a 100 base ssODN comprising 50 bases of sequence from either side of the expected deletion was included to accommodate the cell to repair its cut DNA and make a productive edit. Inclusion of the 50-50 bp ssODN reduced the amount of product from PCR 1 and PCR 2 further suggesting an increase in deletion frequency. PCR 1 was now reduced compared to the UNT by 45% and PCR 2 was reduced by 57%.

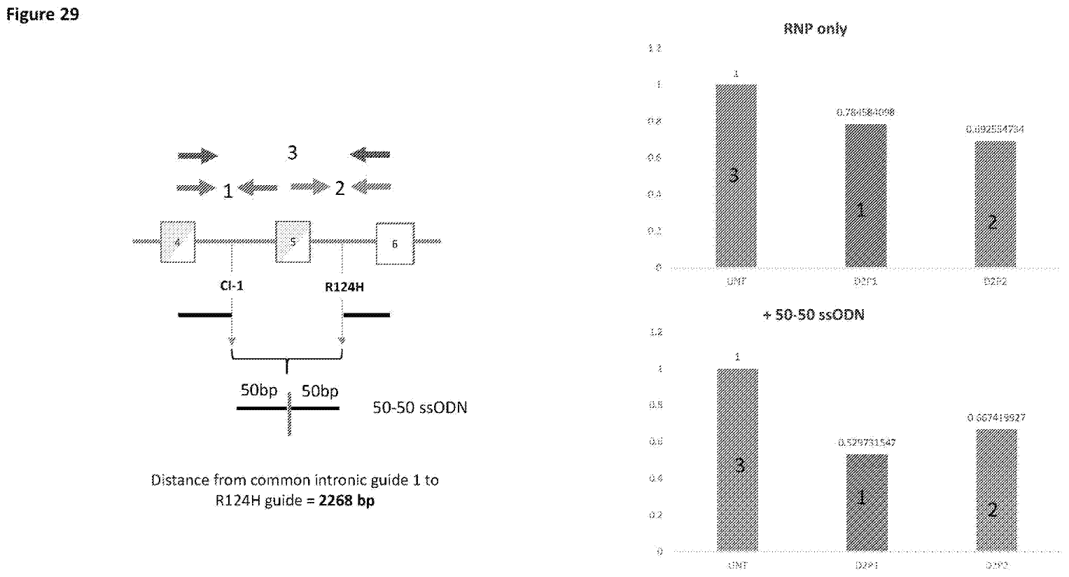

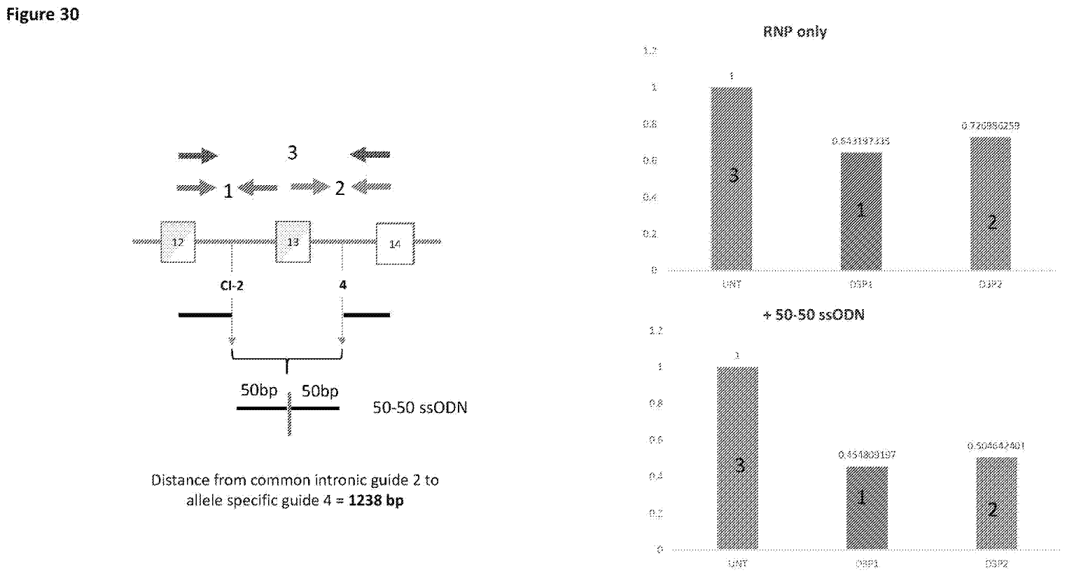

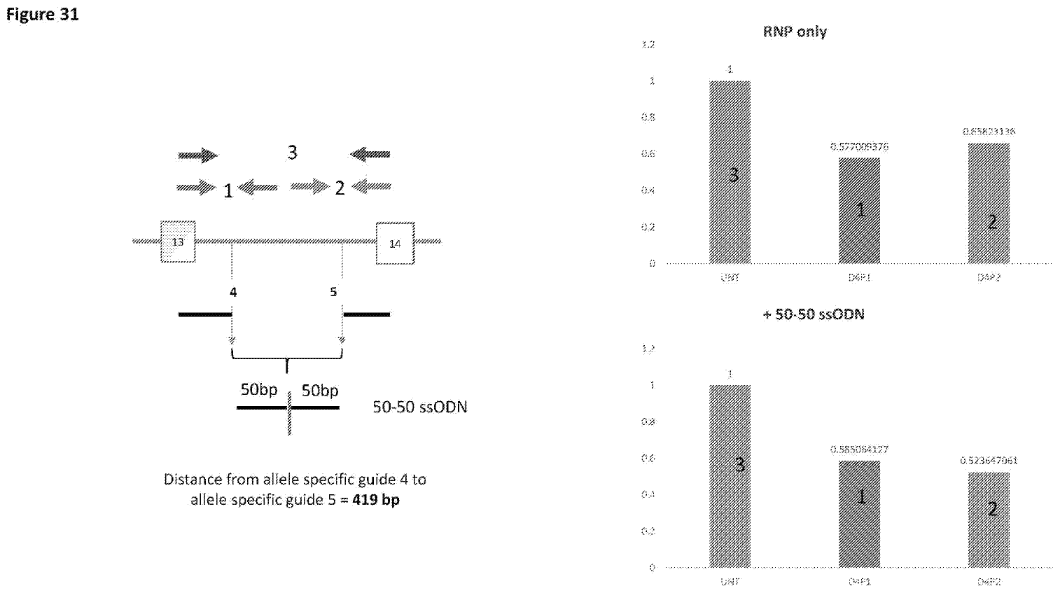

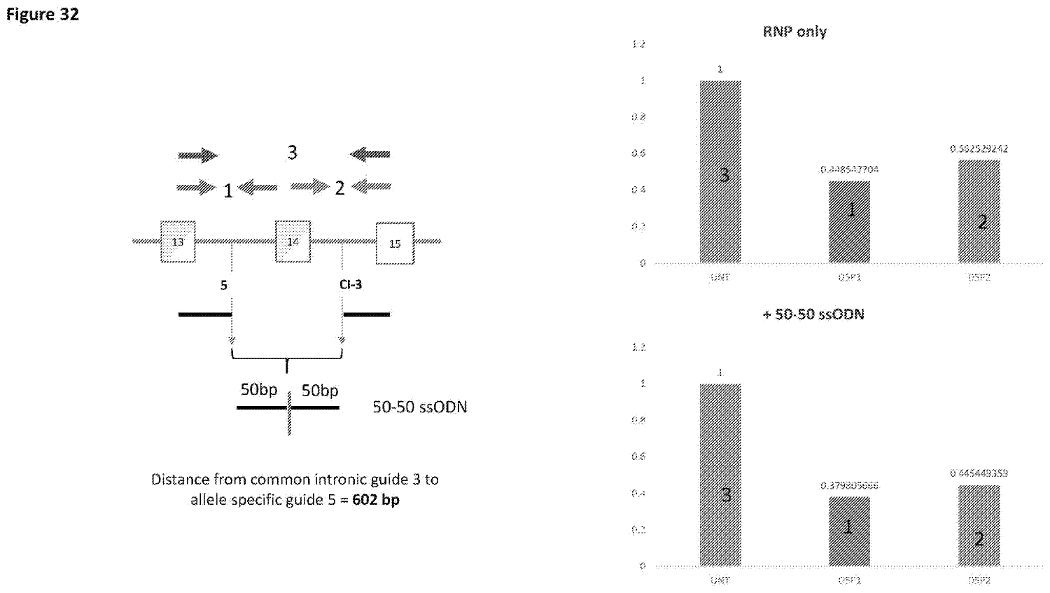

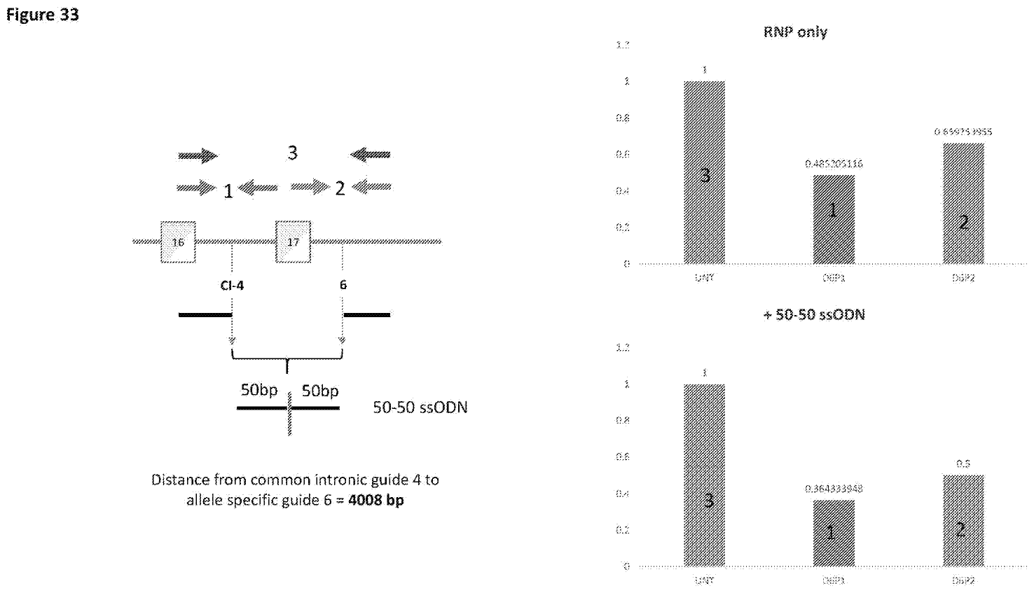

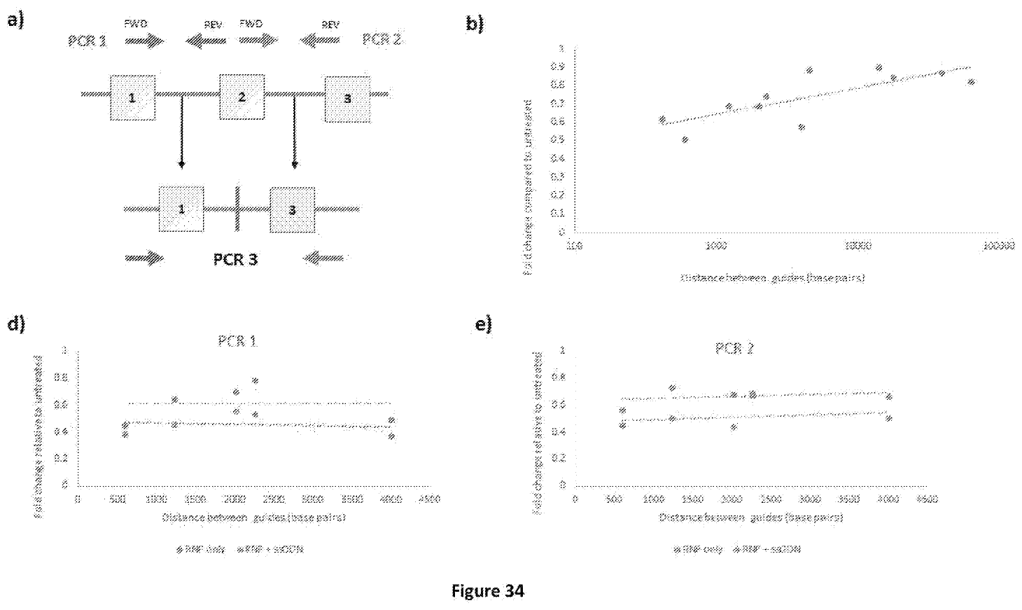

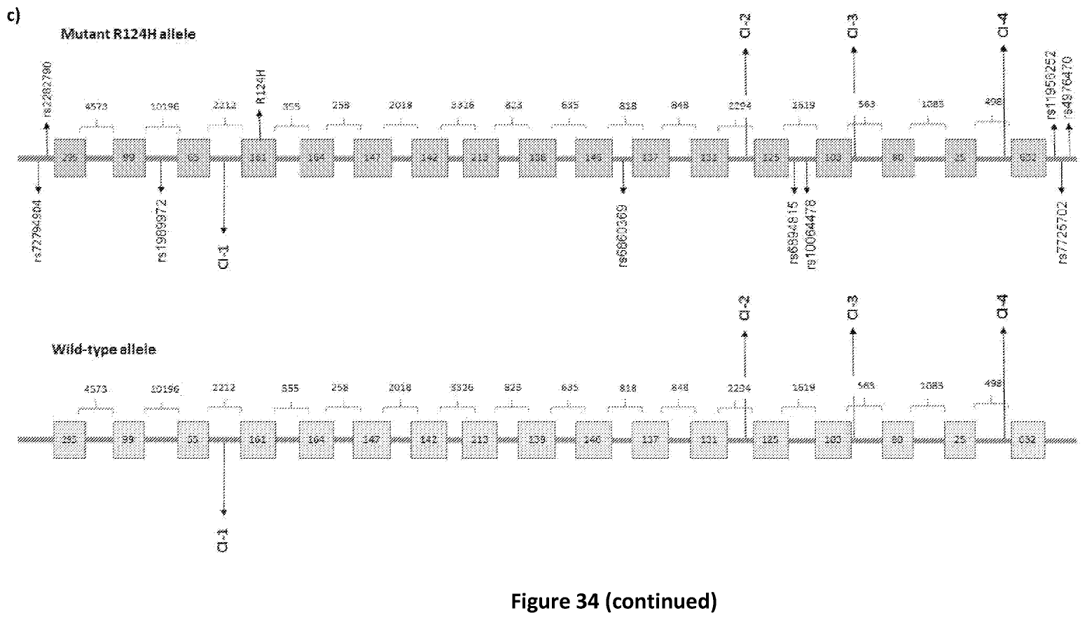

In FIG. 29, PCR 1 was reduced by 22% compared to UNT and PCR 2 was reduced by 31%. With the 50-50 bp ssODN, PCR 1 was reduced compared to the UNT by 53% and PCR 2 was reduced by 33%. In FIG. 30, PCR 1 was reduced by 36% compared to UNT and PCR 2 was reduced by 27%. With the 50-50 bp ssODN, PCR 1 was reduced compared to the UNT by 55% and PCR 2 was reduced by 50%. In FIG. 31, PCR 1 was reduced by 42% compared to UNT and PCR 2 was reduced by 34%. With the 50-50 bp ssODN, PCR 1 was NOT reduced compared to the UNT (41%) and PCR 2 was reduced by 52%. In FIG. 32, PCR 1 was reduced by 55% compared to UNT and PCR 2 was reduced by 44%. With the 50-50 bp ssODN, PCR 1 was reduced compared to the UNT by 62% and PCR 2 was reduced by 54%. In FIG. 33, PCR 1 was reduced by 51% compared to UNT and PCR 2 was reduced by 34%. With the 50-50 bp ssODN, PCR 1 was reduced compared to the UNT by 64% and PCR 2 was reduced by 50%. These data support that the frequency of deletions was enhanced by the addition of deletion spanning (50+50 bp) single-stranded oligonucleotides as shown in FIG. 34. In summary, for the 6 dual combinations tested, in a range of 400-4000 bp apart, none appeared to be notably more efficient. However, in all cases, addition of the 50-50 bp ssODN improved the efficiency of the dual cut.

In some embodiments, a ssODN having a different length (more than 100, such as at least 110 bp, at least 120 bp, at least 130 bp, etc., or less than 100 bp, such as at most 90 bp, at most 80 bp, at most 70 bp, at most 60 bp, at most 50 bp, at most 40 bp) is used.

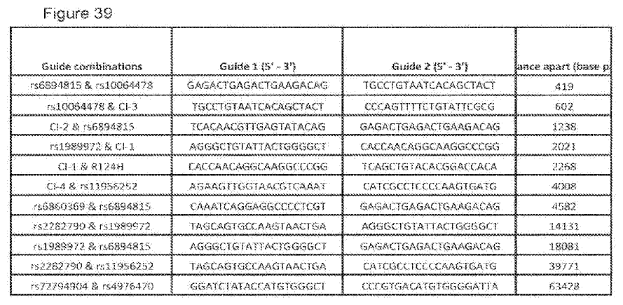

While it was hypothesized that the larger the distance between the dual-guides, the less frequent the deletion would be (as shown in FIG. 34, section b), the deletion frequency remains relatively stable for the distances tested (all dual-combinations used are shown in FIG. 39), ranging from 419 bp to 63,428 bp, (FIG. 34, sections d and e).

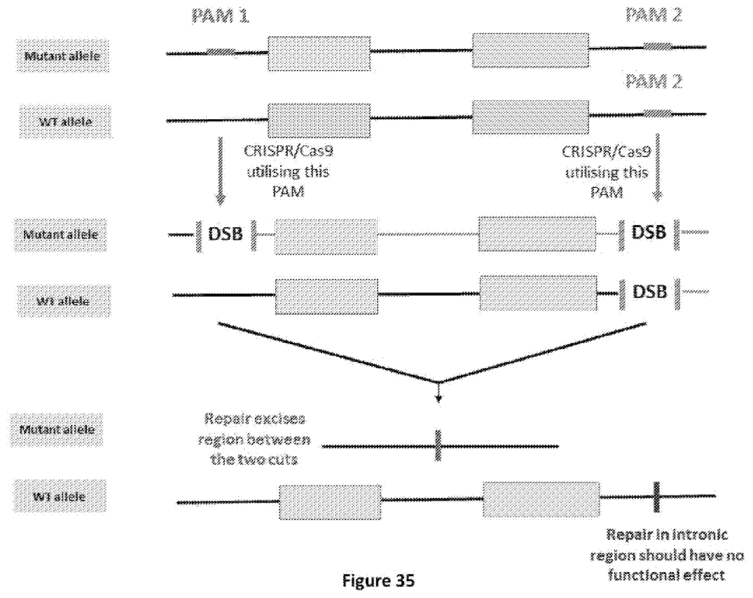

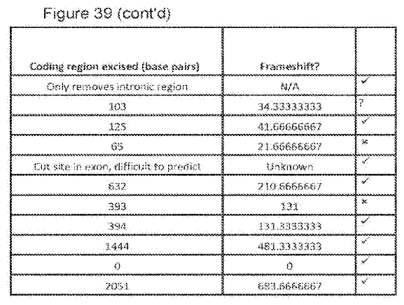

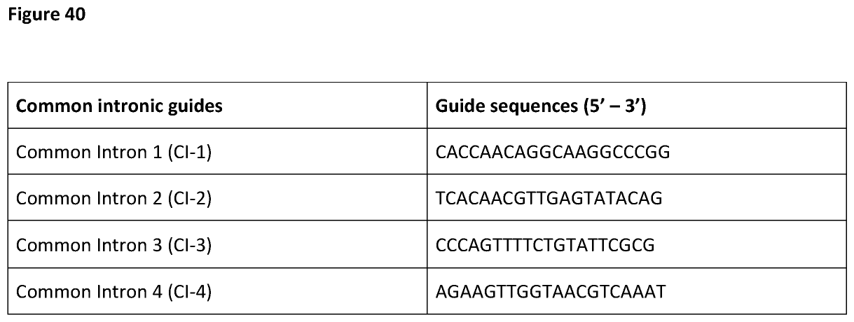

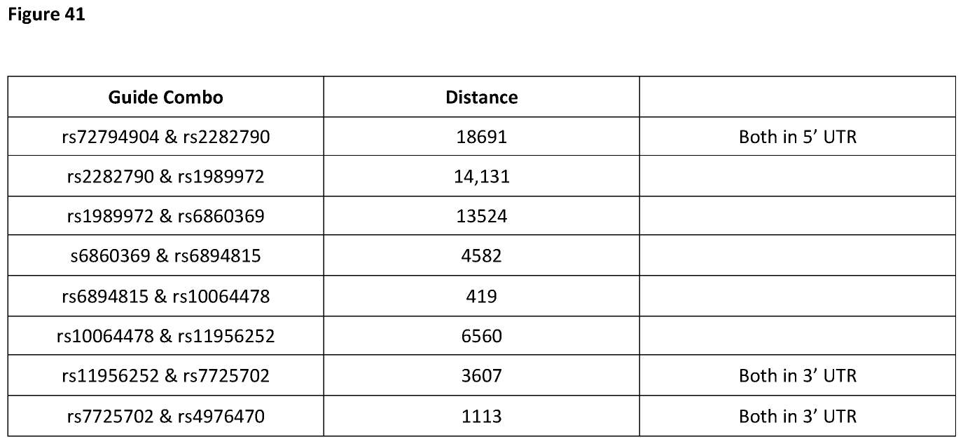

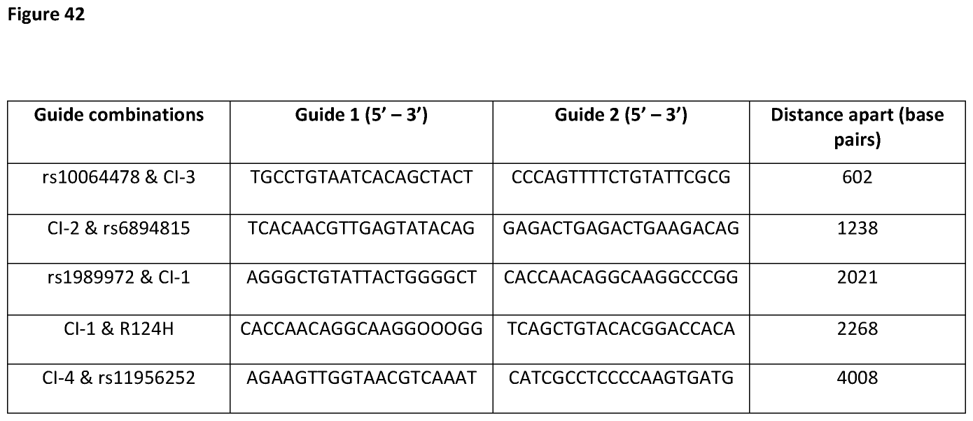

In some cases, the target SNPs described (Table 1) lie substantial distances apart, up to >18 kb (FIG. 40). Additional guides that lie closer to a particular ASNIP guide are used. These additional guides further facilitate excision of exons (FIG. 41) in case the efficiency of deletion drops with the increasing intervening distance. In contrast to the PAM discriminatory guides, these guides are not allele-specific, as they were selected to target the intronic region of both alleles (FIG. 34, section c). The ASNIP guide cuts the mutant allele only while the common-intronic guide cuts both alleles. On the mutant allele when both cuts are made on that chromosome, the region between these cuts may be deleted, while on the wild-type allele, a cut should only occur with the common-intronic guide which at most results in a small indel and should have no functional effect (FIG. 35). The efficiency of the dual-cut was assessed in cells transfected with pairs of RNP complexes; dual combinations with a maximum difference of <3.5 kb, ranging in size from 602 bp to 4008 bp were tested (FIG. 42), in line with previous results we found that small increments in distance had no significant effect on the efficiency of the deletion. On average the reduction of PCR 1 and PCR 2, and hence deletion, when compared to untreated samples, was 38.87%±6.34% for PCR 1 (FIG. 34, section d, shown in blue) and 33.64%±2.76% for PCR 2 (FIG. 34, section e, shown in blue); the variation between reduction efficiencies was not significant and can be attributed to the fact that not all guide sequences perform at equal efficiencies.

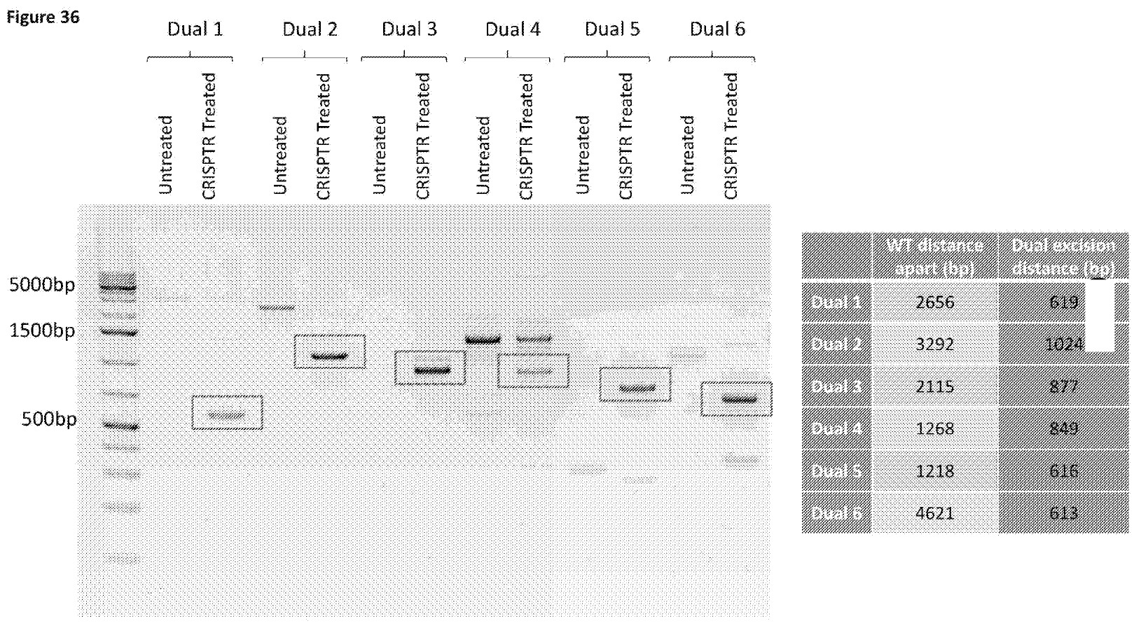

DNA extracted from the R124H cells transfected with various dual combinations was subjected to end point PCR using the PCR 1 forward primer and PCR 2 reverse primer. If the region between the two guides is excised, the PCR will produce band of sizes shown in the table in the right column. As shown in FIG. 36, on the gel, in the untreated lanes, no band of the correct size appeared. However, in the treated samples, bands of the correct size did appear which are indicated by the boxes. The results support that the dual cut indeed occurred.

### Example Materials and Methods

DNA extraction from whole blood and SNP genotyping: DNA was extracted from control blood using the Gentra Puregene Blood Kit (Qiagen) and quantified using a nanodrop. Region of interest was PCR amplified using primer pairs listed in FIG. 38, the PCR product was then purified with the Wizard® PCR Preps DNA Purification System (Promega) and sequenced to determine genotype.

Phased sequencing of R124H patient genome: Genomic DNA was extracted from 3 ml of whole blood with a MagAttract HMW DNA kit (QIAGEN, Hilden, Germany). DNA fragment lengths of approximately 45 kb were enriched for on a Blue Pippen pulsed field electrophoresis instrument (Sage Science, Beverly, Mass., USA). Fragment sizes averaging 51,802 bps were confirmed with a Large Fragment kit on the Fragment Analyzer (Advanced Analytical, Ankeny, Iowa, USA). This high molecular weight (HMW) DNA (1 ng) was partitioned across approximately 1 million synthetic barcodes (GEMs) on a microfluidic Genome Chip with A Chromium™ System (10× Genomics, Pleasanton, Calif., USA) according to the manufacturer's protocol. Upon dissolution of the Genome Gel Bead in the GEM, HMW DNA fragments with 16-bp 10× Barcodes along with attached sequencing primers were released. A standard library prep was performed according to the manufacturer's instructions resulting in sample-indexed libraries using 10× Genomics adaptors. Prior to Illumina bridge amplification and sequencing, the libraries were analyzed on the Fragment Analyzer with the high sensitivity NGS kit. One lane of whole genome paired end short read (2×150 nt) sequencing was conducted on a HiSeq 4000 (Illumina, San Diego, Calif., USA). The FASTQ files served as input into Long Ranger (10× Genomics) which was used to assemble, align and give haplotype phasing information.

In vitro digestion to determine on-target specificity: A double-stranded DNA template was prepared by amplifying a region of the luciferase reporter plasmid containing the desired sequence using the primers listed in FIG. 38.

A cleavage reaction was set up by incubating 30 nM S. pyogenes Cas9 nuclease (NEB UK) with 30 nM synthetic sgRNA (Synthego) for 10 minutes at 25° C. The Cas9:sgRNA complex was then incubated with 3 nM of DNA template at 37° C. for 1 hour. Fragment analysis was then carried out on a 1% agarose gel.

Preparation of primary human PBMCs: A whole blood sample was collected from a patient with Avellino corneal dystrophy. PBMCs were isolated by centrifugation on a Ficoll density gradient. PBMCs were washed in RPMI 1640 media containing 20% FBS and incubated with EBV at 37° C. for 1 hour. After infection RPMI 1640 containing 20% FBS was added to a total volume of 3 ml and 40 μl of 1 mg/ml phytohaemagglutinin was added. 1.5 ml of the lymphocyte mixture was added to two wells of a 24-well plate and allowed to aggregate. Lymphoblastoids were cultured in RPMI 1640 media containing 20% FBS.

Nucleofection of LCLs with RNPs: S. pyogenes Cas9 nuclease (NEB) and modified synthetic sgRNAs (Synthego) were complexed to form RNPs. RNPs were formed directly in the Lonza Nucleofector SF solution (SF Cell line 4D-Nucleofector X kit—Lonza), and incubated for 10 minutes at room temperature. Desired number of cells were spun down (300 g×5 mins) and resuspended in Nucleofector solution. 5 μl of each cell solution was added to 25 μL of corresponding preformed RNPs, mixed and transferred to the nucleofector 16-well strip. The cells were electroporated using the 4D Nucleofector (Lonza) and program DN-100, cells were allowed to recover at room temperature for 5 mins and 70 μl of pre-warmed media was added to each well of Lonza strip to help recovery. The transfected cells were then transferred to 24-well plate with 200 μl media. After 48 hrs of incubation at 37° C., gDNA was extracted using the QIAmp DNA Mini Kit (Qiagen), the target region was PCR amplified using primer pairs listed in FIG. 38 and sequencing data was analysed using Synthego's ICE tool.

Quantitative PCR: RT-qPCRs were performed using 1× LightCycler 480 SYBR Green I Master (Roche), 10 μM primers and 10 ng gDNA. Reactions were run on the LightCycler 480 II (Roche), with an initial incubation step of 95° C., 10 minutes; followed by 45 cycles of 95° C. for 10 seconds, 60° C. for 10 seconds and 72° C. for 10 seconds. Expression was normalized to β-actin, and relative expression was determined using the ΔΔCT method.

1. A method of altering expression of a gene product, the method comprising:
administering into a cell an engineered CRISPR/Cas9 system comprising at least one vector comprising:
(i) a nucleotide molecule encoding Cas9 nuclease;
(ii) a first sgRNA comprising a first CRISPR targeting RNA (crRNA) sequence that hybridizes to a nucleotide sequence complementary to a first target sequence, the first target sequence being adjacent to the 5′-end of a first protospacer adjacent motif (PAM) in a first intron at 3′-end side of an exon comprising a disease-causing mutation or SNP in cis, wherein the first target sequence or the first PAM comprises a first ancestral variation or SNP site; and
(iii) a second sgRNA comprising a second crRNA sequence that hybridizes to a nucleotide sequence complementary to a second target sequence, the second target sequence being adjacent to the 5′-end of a second PAM in a second intron at 5′-end side of the exon comprising the disease-causing mutation or SNP in cis, wherein the second target sequence or the second PAM comprises a second ancestral variation or SNP site,
wherein the at least one vector does not have a nucleotide molecule encoding Cas9 nuclease and a crRNA sequence that naturally occur together.

2. The method of claim 1, wherein at least one of the first and second crRNA sequences comprises a nucleotide sequence selected from the group consisting of guide sequences shown in Table 3.

3. The method of claim 1, wherein
the first crRNA sequence comprises the first target sequence;
the second crRNA sequence comprises the second target sequence;
the first crRNA sequence is from 17 to 24 nucleotide long; and/or
the second crRNA sequence is from 17 to 24 nucleotide long.

4. The method of claim 1, wherein the first and/or second PAMs and the Cas9 nuclease are from Streptococcus or Staphylococcus.

5. The method of claim 1, wherein the first and second PAMs are both from Streptococcus or Staphylococcus.

6. The method of claim 1, wherein each of the first and second PAMs independently consists of NGG or NNGRRT, wherein N is any of A, T, G, and C, and R is A or G.

7. The method of claim 1, wherein the administering comprises injecting the engineered CRISPR/Cas9 system into the cell.

8. The method of claim 1, wherein the administering comprises introducing the engineered CRISPR/Cas9 system into a cell containing and expressing a DNA molecule having the target sequence.

9. The method of claim 1, wherein
the disease is associated with the SNP;
the first target sequence or the first PAM comprises the first ancestral SNP site; and/or
the second target sequence or the second PAM comprises the second ancestral SNP site.

10. The method of claim 1, wherein the target sequence or the PAM comprises a plurality of mutation or SNP sites.

11. The method of claim 1, including:
administering the engineered CRISPR/Cas9 system into a subject.

12. The method of claim 11, wherein the subject is a human.

13. The method of claim 11, further comprising:
prior to administering to the subject the engineered CRISPR/Cas9 system:
obtaining sequence information of the subject; and
selecting the first crRNA sequence and/or the second crRNA sequence based on the sequence information of the subject.

14. The method of claim 13, wherein:
the sequence information of the subject includes whole-genome sequence information of the subject.

15. The method of claim 1, wherein:
the first crRNA sequence hybridizes to the nucleotide sequence so that the Cas9 nuclease cleaves at a first cleaving site that is adjacent to the first ancestral variation or SNP site; and/or
the second crRNA sequence hybridizes to the nucleotide sequence so that the Cas9 nuclease cleaves at a second cleaving site that is adjacent to the second ancestral variation or SNP site.

16. The method of claim 15, wherein:
the first crRNA sequence hybridizes to the nucleotide sequence so that the Cas9 nuclease cleaves only at the first cleaving site that is adjacent to the first ancestral variation or SNP site; and/or
the second crRNA sequence hybridizes to the nucleotide sequence so that the Cas9 nuclease cleaves only at the second cleaving site that is adjacent to the second ancestral variation or SNP site.

17. The method of claim 1, wherein:
the first crRNA sequence hybridizes to the nucleotide sequence complementary to the first target sequence in trans with the disease-causing mutation or SNP, said first target sequence in trans not being adjacent to the 5′-end of a PAM; and/or
the second crRNA sequence hybridizes to the nucleotide sequence complementary to the second target sequence in trans with the disease-causing mutation or SNP, said second target sequence in trans not being adjacent to the 5′-end of a PAM.

18. The method of claim 1, wherein:
the first crRNA sequence hybridizes to the nucleotide sequence complementary to the first target sequence in trans with the disease-causing mutation or SNP, said first target sequence in trans not being adjacent to the 5′-end of a PAM; and
the second crRNA sequence hybridizes to the nucleotide sequence complementary to the second target sequence in trans with the disease-causing mutation or SNP, said second target sequence in trans being adjacent to the 5′-end of a PAM.

19. The method of claim 1, wherein:
the first crRNA sequence hybridizes to the nucleotide sequence complementary to the first target sequence in trans with the disease-causing mutation or SNP, said first target sequence in trans being adjacent to the 5′-end of a PAM; and
the second crRNA sequence hybridizes to the nucleotide sequence complementary to the second target sequence in trans with the disease-causing mutation or SNP, said second target sequence in trans not being adjacent to the 5′-end of a PAM.
